(19) United States
(12) Patent Application Publication
(10) Pub. No.: US 2023/0104171 A1
(43) Pub. Date: Apr. 6, 2023

(54) Engineered Alum-binding SARS-CoV-2 Immunogens

(71) Applicant: Massachusetts Institute of Technology, Cambridge, MA (US)

(72) Inventors: Kristen Alexandra Rodrigues, Cumberland, RI (US); Sergio A. Rodriguez Aponte, Cambridge, MA (US); Neil Dalvie, Cambridge, MA (US); Darrell Irvine, Arlington, MA (US); J. Christopher Love, Somerville, MA (US)

(21) Appl. No.: 17/816,061

(22) Filed: Jul. 29, 2022

Related U.S. Application Data
(60) Provisional application No. 63/251,604, filed on Oct. 2, 2021.

(51) Int. Cl. A61K 39/215; A61P 31/14; C07K 14/165; A61K 9/51

(52) U.S. Cl. CPC A61K 39/215; A61P 31/14; C07K 14/165; A61K 9/51; A61K 2039/55505

(57) ABSTRACT

Compositions are disclosed that include alum and a SARS-CoV-2 Spike (S) glycoprotein variant covalently bound to the alum via at least one linker having 2-12 phosphoserine residues, where the S glycoprotein variant includes a receptor binding domain (RBD) having a mutation of at least one amino acid residue in an angiotensin-converting enzyme 2 (ACE2) receptor binding motif (RBM) relative to a wild-type RBD, where the residue is (i) hydrophobic; and (ii) within an aggregation-prone region of about 3-15 amino acid residues, and the mutation is a substitution of the hydrophobic residue with a different amino acid residue; as are compositions and vaccines including the compositions, and methods for their use.

### CROSS REFERENCE

This application claims priority to U.S. Provisional Patent Application Ser. No. 63/251,604 filed Oct. 2, 2021, incorporated by reference herein in its entirety.

### FEDERAL FUNDING STATEMENT

This invention was made with government support under Grant Nos. AI144462, CA014051, OD011132, and AI145629 awarded by the National Institutes of Health (NIH), and Grant No. W911NF-18-2-0048 awarded by the Army Research Office (ARO). The government has certain rights in this invention.

### SEQUENCE LISTING STATEMENT

A computer readable form of the Sequence Listing is filed with this application by electronic submission and is incorporated into this application by reference in its entirety. The Sequence Listing is contained in the file created on Jul. 26, 2022 having the file name “21-1147-US.xml” and is 202 kb in size.

### INTRODUCTION

There remains a significant need for strategies to facilitate global SARS-CoV-2 vaccine coverage. To this end, subunit vaccines are attractive for their ability to be produced at low cost, at scale and without the need for ultra-cold storage temperatures, but the global supply of adjuvants for accessible vaccines is unclear. The most common clinical vaccine adjuvant, alum, is well-suited to global vaccination campaigns due to its manufacturability and low cost, but alum has exhibited relatively poor immunogenicity with SARS-CoV-2 subunit vaccines to date. Of equal importance to these practical issues is the ability of vaccines to promote neutralizing responses to SARS-CoV-2 variants that are now circulating globally.

### SUMMARY

In one aspect, the disclosure may provide compositions, comprising:

(a) alum; and

(b) a SARS-CoV-2 Spike (S) glycoprotein variant, wherein the S glycoprotein variant comprises:
(i) a receptor binding domain (RBD) having a mutation of at least one amino acid residue in an angiotensin-converting enzyme 2 (ACE2) receptor binding motif (RBM) relative to a wild-type RBD, wherein the residue is (i) hydrophobic; and (ii) within an aggregation-prone region of about 3-15 amino acid residues, wherein the mutation is a substitution of the hydrophobic residue with a different amino acid residue; and
(ii) at least one linker comprising 2-12 phosphoserine residues,
wherein the S glycoprotein variant is covalently bound to the alum via the phosphoserine residues.

In some embodiments, the at least one linker may comprise 2-8 or 2-4 phosphoserine residues. In other embodiments, the at least one linker is present at the N-terminus or C-terminus of the S glycoprotein variant. In further embodiments, the alum may comprise a salt of aluminum. In some embodiments, the alum may comprise aluminum hydroxide, aluminum phosphate, aluminum potassium sulfate, or combinations thereof.

In one embodiment, the hydrophobic residue may have a positive AggScore. In some embodiments, the substitution of the hydrophobic residue may reduce the AggScore of the hydrophobic residue by about 10-100%.

In a further embodiment, the different amino acid residue may be

(i) less hydrophobic;

(ii) found at the same position in a genetic background of at least one species of SARS-CoV; or

(iii) a combination of (i) and (ii).

In one embodiment, the S glycoprotein variant may comprise a mutation of at least one additional hydrophobic amino acid in the aggregation-prone region, wherein the mutation is a substitution of the at least one additional hydrophobic residue with a different amino acid residue, optionally wherein the different amino acid residue is less hydrophobic, found at the same position in a genetic background of at least one species of SARS-CoV, or both.

In one embodiment, the S glycoprotein variant may comprise an RBD having a mutation of at least one amino acid residue in a first and/or second aggregation-prone region relative to a wild-type RBD comprising the amino acid sequence of SEQ ID NO: 1, wherein the first aggregation-prone region comprises amino acid residues 122-126 of SEQ ID NO: 1, and the second aggregation-prone region comprises amino acid residues 158-162 of SEQ ID NO: 1, and wherein the mutation is a substitution with a different amino acid residue.

In other embodiments, the S glycoprotein variant may comprise a RBD comprising a mutation of at least one amino acid residue in an ACE2 RBM relative to a wild-type RBD comprising the amino acid sequence of SEQ ID NO: 1, wherein the amino acid residue is L122 of SEQ ID NO: 1, and optionally F160 of SEQ ID NO: 1, and wherein the mutation is a substitution with a different amino acid residue.

In other embodiments, the compositions may further comprise a non-liposome, non-micelle particle, wherein the particle comprises a lipid, a sterol, a saponin, and an optional additional non-alum adjuvant, wherein the particle is optionally bound to the alum. In one embodiment, the alum and the particle may be bound. In another embodiment, the particle may be covalently bound to the alum via phosphate residues in the particle.

In another embodiment, the disclosure provides pharmaceutical compositions and/or vaccines comprising the composition of embodiment of the disclosure and a pharmaceutically acceptable carrier.

In one embodiment, the disclosure provides methods for generating an immune response against a S glycoprotein variant, comprising administering to a subject an amount effective to generate an immune response in the subject of the composition or vaccine of any embodiment of the disclosure. In another embodiment, the disclosure provides methods of treating a subject in need thereof comprising administering to a subject infected with SARS-CoV-2 the composition or vaccine of any embodiment of the disclosure in an effective amount to induce an immune response against the S glycoprotein variant. In a further embodiment, the disclosure provides methods of limiting SARS-CoV-2 infection in a subject comprising administering to a subject at risk for being exposed to and/or infected by SARS-CoV-2 the composition or vaccine of any embodiment of the disclosure in an effective amount to induce an immune response against the S glycoprotein variant.

In one embodiment, the disclosure provides nucleic acids encoding the S glycoprotein variant and at least one linker comprising 2-12 phosphoserine residues as described in any embodiment herein, expression vectors comprising such nucleic acids operatively linked to a suitable control sequence, and host cells comprising the nucleic acid or expression vector.

### DESCRIPTION OF THE FIGURES

FIG. 1. pSer-modification of SARS-CoV-2 RBD immunogens facilitates binding to alum with retention of key structural epitopes. (A) RBD antigens with phosphoserine peptides conjugated at the N- (pSer₄-RBD) (SEQ ID NO: 120-RBD) or C- (RBD-pSer₄) (RBD-SEQ ID NO: 120) terminus were assayed for phosphates per protein by a malachite green assay. (B) pSer-conjugated or unmodified RBD were mixed with alum, and the fraction of protein bound to alum was assessed before (“Loading”) and after incubation for 24 hours in 10% mouse serum at 37° C. Statistical significance was determined by one-way ANOVA followed by Tukey's post-hoc test. (C) Schematic of modified sandwich ELISA to analyze the antigenicity profile of free RBD (left) or RBD bound to alum-coated plates (right). (D-F) Shown are binding profiles of hACE2-Fc (D), CR3022 (E), H4 (F), and B38 (G) to RBDs captured on anti-histag or alum-coated plates (n=3 replicates), and the area under individual binding curves normalized to unmodified RBD (H). Dashed line indicates signal equivalent to unmodified RBD. Statistical significance was determined by Mann-Whitney test. Values plotted are means±standard deviation. ns p>0.05, **** p<0.0001.

FIG. 2. pSer modification enhances the immunogenicity of alum-adsorbed RBD in mice. BALB/c mice (n=5 animals/group) were immunized with 10 μg unmodified or N- or C-terminal pSer₄-conjugated RBD (N- or C-terminal conjugated RBD to SEQ ID NO: 120) in 50 μg Alhydrogel and boosted at 6 weeks. (A) Serum IgG responses were assessed longitudinally by ELISA. Arrows indicate immunization time points. Values plotted are geometric means±geometric standard deviation. (B) Individual mouse IgG responses from selected time points. Values plotted are geometric means±geometric standard deviation. Statistical significance was determined by two-way ANOVA followed by Tukey's post-hoc test. SARS-CoV-2 pseudovirus neutralizing titer (half-maximal, PSV NT₅₀) (C) and NT₅₀ (D) were assessed for serum collected at day 21 and day 56. The dashed line indicates the limit of detection. Values plotted are means standard deviation. Statistical significance was determined by two-way ANOVA followed by Tukey's post-hoc test. (E) RBD-specific antibody secreting cells (ASCs) in the bone marrow were assessed by ELISPOT at day 112. Representative ELISPOT plate images are shown. Values plotted are means±standard deviation. Statistical significance was determined by one-way ANOVA followed by Tukey's post-hoc test. ns p>0.05, * p<0.05, ** p<0.01.

FIG. 3. pSer-conjugated mutant RBDs elicit potent germinal center responses and neutralizing antibodies in mice. (A, B) Mice (n=4/group) were immunized with 10 μg labeled unmodified or pSer-conjugated RBDJ plus 100 μg alum and injection site fluorescence was tracked longitudinally by IVIS imaging. Shown are whole-animal images (A) and fluorescence quantification (B, means±SD). (C-G) Mice (n=5/group) were immunized and GC and T_{FH} responses in dLNs were analyzed by flow cytometry. Shown are representative gating of RBD-specific GC B cells (C), total GC B cell counts (D), RBD-specific GC B cell counts (E), percent RBD-specific GC B cells (F), and T_{FH} enumeration at day 14 (G). Shown are means±SEM. (H) Mice (n=5/group) were immunized twice (indicated by arrows), and serum antibody responses (geometric means±geometric SD) were tracked by ELISA. (I) Mice (n=5/group) were immunized with varying antigen densities on alum. pSer₄-RBDJ (SEQ ID NO: 120-RBDJ) was loaded on alum at the indicated ratios; all groups received 200 μg alum. Shown are half-maximal pseudovirus neutralization titers (PSV NT₅₀); dashed line indicates LOD. Shown are means±SD.

Statistical significance was determined by two-way ANOVA followed by Tukey's post-hoc test (B, D-G) or Sidak's multiple comparisons test (I). ns p>0.05, * p<0.05, ** p<0.01, *** p<0.001, **** p<00.0001.

FIG. 4. Combining pSer-RBD with alum-binding co-adjuvants enhances humoral immunity. (A) CpG or SMNP were added to alum for 30 min and the fraction of alum-bound adjuvant was measured. (B) The fraction of pSer₄-RBDJ (SEQ ID NO: 120-RBDJ) binding to alum co-loaded with CpG or SMNP was assessed before (“Loading”) and after 24 hours incubation (10% mouse serum, 37° C.). (C-D) Mice (n=3/group) were immunized with 30 μg labeled CpG (C) or 5 μg labeled SMNP (D) with 10 μg RBDJ±100 μg alum and injection site fluorescence was assessed by IVIS. (E-J) Mice (n=5/group) were immunized twice (indicated by arrows) with RBDJ combined with the indicated adjuvants. Shown are serum IgG titers over time (E, G), total IgG from individual animals on day 42 (F), antibody titers by isotype on indicated days (H), Half-maximal inhibitory titers (ID₅₀) values assessed for hACE2-RBD binding in the presence of indicated sera (I), and half-maximal pseudovirus neutralizing titers (PSV NT₅₀, J). Dashed line indicates the LOD. Shown are means±SD (A-D,J) or geometric means±geometric SD (E-I). Statistical significance was determined by one-way ANOVA followed by Tukey's post-hoc test (A,B,E,F,H,I) or Sidak's multiple comparison test (C,D,J). ns p>0.05, * p<0.05, ** p<0.01, *** p<0.001, **** p<0.0001.

FIG. 5. pSer-modification of RBD antigens facilitates anchoring to alum. (A) RBD antigens were expressed with terminal cysteines which can be coupled to short peptide linkers consisting of an N-terminal maleimide group and C-terminal pSer residues separated by a 6-unit poly(ethylene glycol) spacer. (B) pSer-modified RBD antigens are anchored to alum via ligand exchange between the phosphates in the pSer residues and hydroxyls on the surface of alum.

FIG. 6. pSer valency enables tuning of antigen-alum binding and influences humoral immune responses. (A) RBDJ antigens with pSer₄ (SEQ ID NO: 120) or pSer₈ (SEQ ID NO: 121) peptides conjugated at the N-terminus were assayed for phosphates per protein by a malachite green assay. Statistical significance was determined by one-way ANOVA followed by Tukey's post-hoc test. (B) Unmodified, pSer₄-, or pSer₈-conjugated RBDJ (SEQ ID NO: 120 or 121, or conjugated to RBDJ) were mixed with alum, and the fraction of protein bound to alum was assessed before (“Loading”) and after incubation for 24 hours in 10% mouse serum at 37° C. Statistical significance was determined by one-way ANOVA followed by Tukey's post-hoc test. (C) Unmodified, pSer₄-, or pSer₈-conjugated RBDJ (SEQ ID NO: 120 or 121 conjugated to RBDJ) were mixed with alum and incubated in 10% mouse serum at 37° C. The fraction of protein bound to alum was assessed longitudinally. Statistical significance was determined by two-way ANOVA followed by Tukey's post-hoc test. (D) A modified sandwich ELISA approach was used to analyze the antigenicity profile of pSer-modified RBDJ. Shown are binding profiles of hACE2-Fc (top left), CR3022 (top right), H4 (bottom left), and B38 (bottom right) to RBDs captured on alum-coated plates (n=3 replicates), and the area under individual binding curves (E). Statistical significance was determined by two-way ANOVA followed by Sidak's multiple comparison test. Values plotted are means±standard deviation. ns p>0.05, * p<0.05, ** p<0.01, * ** p<0.001, **** p<0.0001.

FIG. 7. pSer valency influences germinal center responses. Representative flow cytometry gating plots of (A) RBD-specific germinal center (GC) B cell, and (B) T follicular helper (T_{FH}) cell staining. (C) BALB/c mice (n=5 animals/group) were immunized with 10 μg unmodified, pSer₄-, or pSer₈-conjugated RBDJ (SEQ ID NO: 120 or 121 conjugated to RBDJ) and 100 μg alum, and germinal center (GC) responses in dLNs were analyzed by flow cytometry over time for total GC B cell counts (top), RBD-specific GC B cell counts (middle), and percent RBD-specific GC B cells (bottom) at 9, 14, 21, and 28 days post-immunization. Statistical significance was determined by two-way ANOVA followed by Tukey's post-hoc test. Values plotted are means±standard deviation. ns p>0.05, * p<0.05, ** p<0.01, *** p<0.001, p<0.0001.

FIG. 8. pSer-RBDJ drainage is a combination of antigen-alum complex trafficking and release of antigen from alum at the injection site. (A) BALB/c mice (n=5 animals/group) were immunized with 10 μg unmodified, pSer₄-, or pSer₈-conjugated RBDJ (SEQ ID NO: 120 or 121 conjugated to RBDJ) and 100 μg alum and boosted at 6 weeks. Half-maximal inhibitory titers (ID₅₀) values were assessed for hACE2-RBD interactions at day 35 and day 70. The dashed line indicates the limit of detection. Values plotted are geometric means±geometric standard deviation. Statistical significance was determined by two-way ANOVA followed by Sidak's multiple comparisons test. (B) Mice were immunized with 10 μg fluorescently labeled RBDJ plus alum and the fluorescence at the injection site was quantified longitudinally (n=4 animals/group), as in FIG. 3A-B. The signal remaining at the injection site at day 49 for pSer₈-RBDJ (SEQ ID NO: 121-RBDJ) was subtracted from the longitudinal pSer₈-RBDJ (SEQ ID NO: 121-RBDJ) signal and plotted for comparison to pSer₄-RBDJ (SEQ ID NO: 120-RBDJ). Values plotted are means±standard deviation. Statistical significance between pSer₄-RBDJ and pSer₈-RBDJ (SEQ ID NO: 120-RBDJ and SEQ ID NO:121-RBDJ) was determined by two-way ANOVA followed by Tukey's post-hoc test. (C) Mice were immunized with 10 μg RBDJ plus 100 μg of labeled alum and the fluorescence at the injection site was quantified longitudinally (n=3 animals/group). Values plotted are means±standard deviation. Statistical significance was determined by two-way ANOVA followed by Tukey's post-hoc test. ns p>0.05, * p<0.05, ** p<0.01, *** p<0.001, **** p<0.0001.

FIG. 9. Average antigen density of pSer-RBDJ on alum does not significantly alter humoral responses. (A) pSer₄-RBDJ (SEQ ID NO: 120-RBDJ) was mixed with alum in varying ratios, and the fraction of protein bound to alum was assessed before (“Loading”) and after incubation for 24 hours in 10% mouse serum at 37° C. Values plotted are means±standard deviation. Arrows indicate ratios selected for further evaluation. (B) BALB/c mice (n=5 animals/group) were immunized with 10 μg pSer₄-RBDJ (SEQ ID NO: 120-RBDJ) and varying amounts of alum and boosted at 6 weeks. Serum IgG antibody responses were assessed longitudinally by ELISA. Arrows indicate immunization time points. Values plotted are geometric means±geometric standard deviation. Statistical significance was determined by two-way ANOVA followed by Tukey's post-hoc test. (C) Mice (n=5 animals/group) were immunized with 10 μg unmodified or pSer₄-conjugated RBDJ (SEQ ID NO: 120-RBDJ) and varying amounts of alum, and germinal center (GC) responses in dLNs were analyzed by flow cytometry at day 14 post-immunization. Values plotted are means±standard deviation. Statistical significance was determined by two-way ANOVA followed by Tukey's post-hoc test. (D) Alum loaded with AlexaFluor™647-labeled pSer₄-RBDJ (SEQ ID NO: 120-RBDJ) were mixed with alum labeled with pSer₄-AlexaFluor™488 (SEQ ID NO: 120-AlexaFluor™488) at low density and incubated together for 2 days prior to imaging (left). Representative image shown. The fluorescence overlap was assessed (right) and (E) the fraction of alum particles with fluorescent signal colocalization was measured. Values plotted are means±standard deviation. (F) Mice (n=5 animals/group) were immunized with 10 μg pSer₄-RBDJ (SEQ ID NO: 120-RBDJ) plus 200 μg alum at varying average antigen densities. Immunizations were prepared by first loading pSer₄-RBDJ (SEQ ID NO: 120-RBDJ) on alum at the indicated ratios and then supplementing alum just prior to immunization such that all groups received an equal alum dose. GC responses in dLNs were analyzed by flow cytometry at day 14 post-immunization. Values plotted are means±standard deviation. Statistical significance was determined by two-way ANOVA followed by Tukey's post-hoc test. (G) Mice (n=5 animals/group) were immunized with 10 μg pSer₄-RBDJ (SEQ ID NO: 120-RBDJ) plus 200 μg alum at varying average antigen densities. Serum IgG antibody responses were assessed longitudinally by ELISA. Arrows indicate immunization time points. Values plotted are geometric means±geometric standard deviation. Statistical significance was determined by two-way ANOVA followed by Tukey's post-hoc test. Statistical comparisons between all pSer₄-RBDJ (SEQ ID NO: 120-RBDJ) groups are denoted above plot, while statistical comparison between each pSer₄-RBDJ (SEQ ID NO: 120-RBDJ) group against the RBDJ group is denoted between pSer₄-RBDJ (SEQ ID NO: 120-RBDJ) groups and RBDJ group. (H) Serum SARS-CoV-2 pseudovirus neutralizing titer IDso (PSV NT₈₀) were assessed for serum collected at day 28 and day 56. The dashed line indicates the limit of detection. Values plotted are means±standard deviation. Statistical significance was determined by two-way ANOVA followed by Tukey's post-hoc test. ns p>0.05, * p<0.05, ** p<0.01, *** p<0.001, **** p<0.0001.

FIG. 10. Co-adjuvants SMNP and CpG promote balanced antibody isotype responses and enhance humoral responses. (A) BALB/c mice were immunized with 10 μg fluorescently labeled RBDJ with or without alum plus co-adjuvants CpG or SMNP, and the fluorescence at the injection site was quantified longitudinally (n=3 animals/group). Values plotted are means±standard deviation. Statistical significance between pSer₄-RBDJ (SEQ ID NO: 120-RBDJ) groups was determined by one-way ANOVA followed by Tukey's post-hoc test. (B) Mice (n=5 animals/group) were immunized with 10 μg unmodified RBDJ or pSer₄-RBDJ (SEQ ID NO: 120-RBDJ) and 100 μg alum and/or 30 μg CpG or 5 μg SMNP and boosted at 6 weeks, as in FIG. 4E-G. The ratio of IgG2a to IgG1 (left) and IgG2b to IgG1 (right) were calculated at day 35 and day 70. Values plotted are means±standard deviation. Statistical significance was determined by two-way ANOVA followed by Tukey's post-hoc test. (C) Mice (n=5 animals/group) were immunized with 10 μg unmodified RBDJ or pSer₄-RBDJ (SEQ ID NO: 120-RBDJ) and 100 μg alum and/or 5 μg SMNP and boosted at 6 weeks, as in FIG. 4G. Serum SARS-CoV-2 pseudovirus neutralizing titer IDso (PSV NT₅₀) were assessed for serum collected at day 42 and day 84. The dashed line indicates the limit of detection. Values plotted are means±standard deviation. Statistical significance was determined by two-way ANOVA followed by Tukey's post-hoc test. (D) Mice (n=5 animals/group) were immunized with 10 μg unmodified RBDJ or pSer₄-RBDJ (SEQ ID NO: 120-RBDJ) and 100 μg alum and/or 5 μg SMNP and boosted at 6 weeks, as in FIG. 4G. Plotted is the binding titer versus the neutralizing titer for both day 42 and 84. The corresponding linear fit is plotted as a dashed line, and the Pearson correlation was assessed for each timepoint. ns p>0.05, * p<0.05, ** p<0.01, *** p<0.001, **** p<0.0001.

FIG. 11. Co-adjuvants enhance antigen uptake and germinal center responses BALB/c mice (n=5 animals/group) were immunized with 10 μg AlexaFluor™ 555 labeled antigen and 100 μg alum and 5 μg SMNP or 30 μg CpG, and the inguinal lymph nodes were collected 7 days post-immunization. (A) Representative flow cytometry gating plots. (B) The number of cells positive for AlexaFluor™555 labeled antigen is plotted for B cells, monocytes, neutrophils, subcapsular sinus macrophages, medullary macrophages, and dendritic cells. Values plotted are means±standard deviation. Statistical significance was determined by one-way ANOVA followed by Tukey's post-hoc test. (C) Mice (n=5 animals/group) were immunized with 10 μg unmodified RBDJ or pSer₄-RBDJ (SEQ ID NO: 120-RBDJ) and 100 μg alum and/or 5 μg SMNP and the RBD-specific germinal center (GC) B cell responses in the dLNs were analyzed by flow cytometry at day 14 post-immunization. Values plotted are means±standard error of the mean. Statistical significance was determined by one-way ANOVA followed by Tukey's post-hoc test. ns p>0.05, * p<0.05, ** p<0.01, *** p<0.001, **** p<0.0001.

FIG. 12. Overview of immunization platform and optimization strategy. (A) Outline of the iterations of the immunization platform. From left to right, we first investigated the impact of N- versus C-terminus pSer conjugation to RBD, moving forward with the N-terminal pSer conjugation approach. Next, we assessed the role of pSer valency with an engineered RBD protein called RBDJ and tested the impact of antigen density on alum. Finally, we added alum-binding co-adjuvants to investigate synergistic enhancement of responses. (B) The corresponding post-boost serum SARS-CoV-2 pseudovirus neutralizing titer ID₅₀ (PSV NT₅₀) for relevant groups, reproduced from FIG. 2C, FIG. 3I, and FIG. 4J. The dashed line indicates the limit of detection. Values plotted are means±standard deviation. Statistical significance was determined by two-way ANOVA followed by Tukey's post-hoc test. ns p>0.05, * p<0.05, ** p<0.01, * ** p<0.001, **** p<0.0001.

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

In all embodiments of polypeptides disclosed herein, any N-terminal methionine residues are optional (i.e.: the N-terminal methionine residue may be present or may be absent, and may be included or excluded when determining percent amino acid sequence identity compared to another polypeptide).

In all embodiments of polypeptides disclosed herein, 1, 2, 3, 4, or 5 amino acids may be deleted from the N-terminus and/or the C-terminus so long as function is maintained, and not be considered when determining percent identity.

As used herein, the term “nanoparticle” refers to submicron particles less 100 nm in dimension. In some embodiments, when nanoparticles form aggregates, the size of the aggregates may exceed 100 nm.

As used herein, “about” will mean up to plus or minus 5% of the particular value.

As used herein, the term “adjuvant” refers to any substance that acts to augment and/or direct antigen-specific immune responses when used in combination with specific antigens. When combined with a vaccine antigen, adjuvant increases the immune response to the vaccine antigen as compared to the response induced by the vaccine antigen alone. Adjuvants help drive immunological mechanisms and shape the output immune response to vaccine antigens.

In a first aspect, the disclosure provides compositions, comprising:

(a) alum; and

(b) a SARS-CoV-2 Spike (S) glycoprotein variant, wherein the S glycoprotein variant comprises:
(i) a receptor binding domain (RBD) having a mutation of at least one amino acid residue in an angiotensin-converting enzyme 2 (ACE2) receptor binding motif (RBM) relative to a wild-type RBD, wherein the residue is (i) hydrophobic; and (ii) within an aggregation-prone region of about 3-15 amino acid residues, wherein the mutation is a substitution of the hydrophobic residue with a different amino acid residue; and
(ii) at least one linker comprising 2-12 phosphoserine residues, wherein the S glycoprotein variant is covalently bound to the alum via the phosphoserine residues.

As shown in the studies described herein, compositions of the disclosure provided synergistic enhancements in vaccine immunogenicity.

### Alum

As used herein, alum is any salt of aluminum. In one embodiment, the alum comprises aluminum hydroxide, aluminum phosphate, aluminum potassium sulfate, or combinations thereof. In another embodiment, the alum comprises aluminum hydroxide.

### Phosphoserine Modification

The S glycoprotein variant is covalently bound to the alum via the phosphoserine residues, as described in published US patent application US 20190358312, incorporated by reference herein in its entirety. As used herein, linkers comprising phosphoserine residues are referred herein as “phosphoserine linkers” (PS-linkers). In all embodiments, the linker may comprise any further residues suitable for linking the S glycoprotein variant to the alum. In some embodiment, the PS-linker comprises 1-12 consecutive PS residues followed by a short poly(ethylene glycol) spacer and N-terminal maleimide functional group. In another embodiment, the maleimide functional group at the N-terminal of the PS-linker is covalently via a thioether linkage to a thiol group on the S glycoprotein variant. In yet another embodiment, the multiple PS-linkers are conjugated to an S glycoprotein variant protein via azide functional groups and coupled to a DBCO-modified antigen. The linkers may be employed, for instance, to ensure that an S glycoprotein variant is positioned relative alum to ensure proper folding and formation of the antigen or to block or expose particular epitopes. In one embodiment, the S glycoprotein variant comprises at least one linker comprising 2-12 phosphoserine residues, wherein the S glycoprotein variant is covalently bound to the alum via the phosphoserine residues. In another embodiment, the S glycoprotein variant comprises at least one linker comprising 2-8 phosphoserine residues. In a further embodiment, the S glycoprotein variant comprises at least one linker comprising 2-4 phosphoserine residues. The linker may be present at any suitable position on the S glycoprotein variant; in one embodiment, the at least one linker is present at the N-terminus or C-terminus of the S glycoprotein variant; in some embodiments, the at least one linker is present at the N-terminus of the S glycoprotein variant.

### S-Glycoprotein Variant

The S glycoprotein variant comprises (i) a receptor binding domain (RBD) having a mutation of at least one amino acid residue in an angiotensin-converting enzyme 2 (ACE2) receptor binding motif (RBM) relative to a wild-type RBD, wherein the residue is (i) hydrophobic; and (ii) within an aggregation-prone region of about 3-15 amino acid residues, wherein the mutation is a substitution of the hydrophobic residue with a different amino acid residue.

The COVID19 outbreak caused by the severe acute respiratory syndrome coronavirus 2 (SARS-CoV-2) is a widespread public health threat. SARS-CoV-2 belongs to the family of coronaviridae, a family of viruses (e.g., MERS-CoV and Severe Acute Respiratory Syndrome (SARS-CoV)) that primarily infect the upper respiratory and gastrointestinal tracts of mammals and birds, and that are responsible for acute and chronic diseases of the respiratory, hepatic, gastrointestinal and neurological systems. Coronaviruses are enveloped positive-sense, single-stranded RNA viruses with a nucleocapsid of helical symmetry and virions with a crown-like appearance. The crown-like appearance is due to the club-shaped spike (S) proteins projecting from the surface of the envelope.

The S proteins are responsible for virus binding, fusion and entry, and are inducers of neutralizing antibodies. These proteins play critical roles in viral pathogenesis and virulence. The S protein of SARS-CoV-2 is a type I transmembrane glycoprotein consisting of two domains, S1 and S2. S1 is responsible for virus binding to the receptor on the target cell. It has been demonstrated that angiotensin-converting enzyme 2 (ACE2) is a functional receptor for SARS-CoV-2. A fragment located in the middle region of S1 is the receptor-binding domain (RBD). S2 domain, which contains a putative fusion peptide and two heptad repeat (HR1 and HR2) regions, is responsible for fusion between viral and target cell membranes.

A receptor-binding domain (RBD) of the S protein, containing residues 318-510 (RBD193), was identified in the related SARS-CoV and found to bind to ACE2 in vitro (Wong et al., JBC., 279: 3197-3201 (2004)). In addition, recombinant proteins RBD193 and a related construct, RBD219 (residues 318-536), expressed in the culture supernatant of mammalian cells 293T and Chinese hamster ovary (CHO)-Kl, respectively, were demonstrated to elicit neutralizing antibodies and protective immunity in vaccinated mice (Du et al., Virology., 393(1): 144-150 (2009); Du et al., Viral Immuno., 23(2): 211-219 (2012). Moreover, RBD can also absorb and remove the majority of neutralizing antibodies in the antisera of mice, monkeys, and rabbits immunized with whole SARS-CoV or vaccinia virus expressing S protein constructs (Chen et al., World J Gastroenterol., 11(39):6159-6164 (2005)).

The present compositions incorporate SARS-CoV-2 Spike (S) glycoprotein variants having reduced aggregation, increased thermostability and/or reduced hydrophobicity, thereby resulting in improved expression and/or production in host cells of interest (e.g., Komagataella phaffii). Surprisingly, SARS-CoV-2 S glycoprotein variants were discovered having increased immunogenicity where the variant comprises a mutation of one or more amino acid residues in an ACE2 receptor binding motif (RBM) in the RBD, wherein the residue is (i) hydrophobic; and (ii) within an aggregation-prone region of about 3-15 amino acid residues, and wherein the mutation is a substitution of the hydrophobic residue with a different amino acid residue, e.g., a less hydrophobic residue found in another coronavirus species.

It was also surprisingly discovered that mutating a hydrophobic amino acid residue within an aggregation-prone region in a SARS-CoV-2 RBD to an amino acid residue conserved in at least one coronavirus species (e.g., β-genus coronavirus, e.g., SARS-CoV strains isolated from different hosts and/or in different years), results in improved expression and production. As described herein, aggregation-prone regions and hydrophobic amino acid residues were identified in the RBD of SARS-CoV-2 based on an aggregation score, with the highest scores identified in the ACE2 RBM. The sequences of the aggregation-prone regions in the SARS-CoV-2 RBD were then compared with RBD sequences of previously known SARS-related coronavirus strains (e.g., isolated from human, civet, or bat) to identify conserved, and/or less hydrophobic amino acid residues at the same position as the one or more the identified hydrophobic amino acid residues in the aggregation-prone regions in the SARS-CoV-2 RBD. SARS-CoV-2 S glycoprotein RBD variants were generated by mutating at least one hydrophobic amino acid residue to an amino acid residue conserved amongst other SARS-CoV virus species.

Without being bound by theory, it is believed that because the SARS-CoV-2 spike protein shares substantial sequence identity with the SARS-CoV spike protein, substitution of a hydrophobic residue in the SARS-CoV-2 S glycoprotein with a conserved residue provides a SARS-CoV-2 S glycoprotein variant that maintains ACE2 receptor binding, while resulting in one or more desired properties in the variant (e.g., reduced aggregation, increased thermostability, reduced hydrophobicity) to improve expression and/or production in host cells of interest (e.g., K. phaffii). Moreover, without being bound by theory, it is believed mutating (e.g., substituting) a hydrophobic amino acid residue to a conserved amino acid residue would minimally alter or not alter the overall structure of the protein such that an immune system response directed against the SARS-CoV-2 S glycoprotein variant (e.g., neutralizing antibodies against the SARS-CoV-2 S glycoprotein variant) will likewise recognize the wild-type SARS-CoV-2 S glycoprotein.

As described herein, expression of SARS-CoV-2 S glycoprotein variants having a substitution of at least one hydrophobic amino acid residue within an aggregation-prone region resulted in reduced aggregation of the SARS-CoV-2 S glycoprotein variant, and improved expression in a host cell of interest, e.g., K. phaffii. Without wishing to be bound by theory, reduced aggregation during expression is expected to improve the scalability and ease of manufacture of vaccines by recombinant methods in host cells of interest, e.g., K. phaffii, and reduced overall cost of manufacture. Surprisingly, the SARS-CoV-2 S glycoprotein variants described herein not only had higher expression levels, but also induced higher levels of IgG neutralizing antibodies in vivo.

Accordingly, in some aspects, the compositions include a SARS-CoV-2 S glycoprotein variant, wherein the S glycoprotein variant comprises a receptor binding domain (RBD) having a mutation of at least one amino acid residue in an ACE2 receptor binding motif (RBM) relative to a wild-type RBD, wherein the residue is (i) hydrophobic; and (ii) within an aggregation-prone region of about 3-15 amino acid residues, wherein the mutation is a substitution of the hydrophobic residue with a different amino acid residue. In some aspects, the S glycoprotein variant comprises a mutation of at least one additional hydrophobic amino acid in the aggregation-prone region, wherein the mutation is a substitution of the at least one additional hydrophobic residue with a different amino acid residue. In some aspects, the S glycoprotein variant comprises a mutation of at least one hydrophobic amino acid in a second aggregation-prone region of about 3-15 amino acid residues, and wherein the mutation is a substitution of the at least one additional hydrophobic residue with a different amino acid residue. In some aspects, the second aggregation-prone region is outside of the ACE2 RBM. In any of the foregoing or related aspects, the RBD comprises at least one mutation to at an asparagine-linked glycosylation site relative to the wild-type RBD.

In any of the foregoing or related aspects, the different amino acid residue is less hydrophobic. In some aspects, the different amino acid residue is an amino acid residue that is found at the same position in a genetic background of at least one species of SARS-CoV. In some aspects, the different amino acid residue is an amino acid residue that is found at the same position in a genetic background of at least one species of SARS-CoV and is less hydrophobic.

In any of the foregoing or related aspects, the hydrophobic residue has a positive AggScore. In some aspects, the hydrophobic residue has an AggScore of at least 2, or of about 2-10, 5-10, 10-15, or 15-20. In some aspects, the substitution of the hydrophobic residue reduces the AggScore of the hydrophobic residue by about 10-100%. In some aspects, the substitution of the hydrophobic residue reduces the overall aggregation score of the aggregation prone region by about 5-50% relative to the aggregation prone region without the substitution. In some aspects, the substitution of the hydrophobic residue reduces the overall aggregation score of the S glycoprotein variant by about 5-50% relative to the S glycoprotein variant without the substitution.

The term “AggScore” or “aggregation score” refers to measurement determined by analyzing the distribution of hydrophobic and electrostatic patches on the surface of a protein, factoring in the intensity and relative orientation of the respective surface patches into an aggregation propensity function that has been trained on a benchmark set of 31 adnectin proteins. AggScore can accurately identify aggregation-prone regions in several well-studied proteins and also reliably predict changes in aggregation behavior upon residue mutation.

In any of the foregoing or related aspects, the substitution of the hydrophobic residue reduces the propensity of the SARS-CoV-2 S glycoprotein to aggregate compared to the SARS-CoV-2 S glycoprotein without the substitution. In some aspects, the substitution of the hydrophobic residue increases the thermostability of the S glycoprotein compared to the SARS-CoV-2 S glycoprotein without the substitution.

In some aspects, the compositions include a SARS-CoV-2 S glycoprotein variant, wherein the S glycoprotein variant comprises an RBD having a mutation of at least one amino acid residue in a first and/or second aggregation-prone region relative to a wild-type RBD comprising the amino acid sequence of SEQ ID NO: 1 (RBD sequence), wherein the first aggregation-prone region comprises amino acid residues 122-126 of SEQ ID NO: 1, and the second aggregation-prone region comprises amino acid residues 158-162 of SEQ ID NO: 1, and wherein the mutation is a substitution with a different amino acid residue. See Tables 1-2 for residue numbering conversion between the RBD sequence and full length S protein sequence.

TABLE 1
Contact Residues of SARS-CoV-2
RBD when bound to hACE2*

| | Residue | Corresponding |
|---|---|---|
| | number of | residue |
| | the full-length | number of the |
| | S glycoprotein | wild-type RBD |
| Contact | set forth by | set forth by |
| Residue | SEQ ID NO: 59 | SEQ ID NO: 1 |
| K | 417 | 87 |
| G | 446 | 116 |
| Y | 449 | 119 |
| Y | 453 | 123 |
| L | 455 | 125 |
| F | 456 | 126 |
| A | 475 | 145 |
| F | 486 | 156 |
| N | 487 | 157 |
| Y | 489 | 159 |
| Q | 493 | 163 |
| G | 496 | 166 |
| Q | 498 | 168 |
| T | 500 | 170 |
| N | 501 | 171 |
| G | 502 | 172 |
| Y | 505 | 175 |

*Reference: Lan et al (2020) NATURE 581:215

TABLE 2
Conserved Mutations of Amino Acid Residue in
Aggregation-Prone Regions of SARS-CoP-2 S Glycoprotein

| | | Position | | |
|---|---|---|---|---|
| | | based | | |
| | Position | on full- | | |
| Residue | based on | length | | |
| of Wild- | SARS- | SARS- | | SARS-CoV |
| type | CoV-2 | CoV-2 | | Species |
| SARS- | RBD | S protein | | Source(s) for |
| CoV-2 | (SEQ ID | (SEQ ID | Amino Acid | Conserved |
| S protein | NO: 1) | NO: 59) | Substitution | Substitution |
| L | 38 | 368 | F | Bat SARS CoV |
| L | 122 | 452 | K | Bat SARS CoV, |
| | | | | Civet SARS CoV, |
| | | | | Human SARS CoV, |
| L | 122 | 452 | F | Bat SARS CoV |
| L | 122 | 452 | Y | Bat SARS CoV |
| L | 122 | 452 | S | MERS CoV |
| L | 125 | 455 | S | Bat SARS CoV |
| L | 125 | 455 | Y | Civet SARS CoV, |
| | | | | Human SARS CoV |
| L | 125 | 455 | W | Bat SARS CoV |
| L | 125 | 455 | N | MERS CoV |
| F | 126 | 456 | L | Bat SARS CoV, |
| | | | | Civet SARS CoV, |
| | | | | Human SARS CoV |
| F | 126 | 456 | H | Bat SARS CoV |
| F | 126 | 456 | V | Bat SARS CoV |
| F | 126 | 456 | K | MERS CoV |
| C | 158 | 488 | G | Bat SARS CoV |
| C | 158 | 488 | T | MERS CoV |
| Y | 159 | 489 | V | Bat SARS CoV, |
| | | | | MERS CoV |
| Y | 159 | 489 | A | Bat SARS CoV |
| F | 160 | 489 | W | Bat SARS 2013, |
| | | | | Civet SARS CoV, |
| | | | | Human SARS CoV |
| F | 160 | 490 | R | Bat SARS CoV |
| F | 160 | 490 | Y | Bat SARS CoV |
| F | 160 | 490 | N | Bat SARS CoV |
| L | 187 | 517 | I | MERS CoV |
| L | 188 | 518 | T | MERS CoV |

In some aspects, the S glycoprotein variant comprises an RBD having a mutation of at least one amino acid residue in the first aggregation-prone region relative to the wild-type RBD comprising the amino acid sequence of SEQ ID NO: 1 (see Table 3 below), wherein the first aggregation-prone region comprises amino acid residues 122-126 of SEQ ID NO: 1, and wherein the mutation is a substitution with a different amino acid residue. In some aspects, the S glycoprotein variant comprises an RBD having a mutation of at least one amino acid residue in the second aggregation-prone region relative to the wild-type RBD comprising the amino acid sequence of SEQ ID NO: 1, wherein the second aggregation-prone region comprises amino acid residues 158-162 of SEQ ID NO: 1, and wherein the mutation is a substitution with a different amino acid residue. In some aspects, the S glycoprotein variant comprises an RBD having a mutation of at least one amino acid residue in the first and the second aggregation-prone region relative to the wild-type RBD comprising the amino acid sequence of SEQ ID NO: 1, wherein the first aggregation-prone region comprises amino acid residues 122-126 of SEQ ID NO: 1, and the second aggregation-prone region comprises amino acid residues 158-162 of SEQ ID NO: 1, and wherein the mutation is a substitution with a different amino acid residue. In some aspects, the at least one amino acid residue is selected from: L122, L125, F126, Y159, F160, and any combination thereof. In some aspects, the S glycoprotein variant comprises an amino acid substitution at L122 with a different amino acid residue. In some aspects, the S glycoprotein variant comprises an amino acid substitution at L122 and F160 with a different amino acid residue. In some aspects, the S glycoprotein variant comprises an amino acid substitution at L122, L125, F126 and F160 with a different amino acid residue. In some aspects, the different amino acid residue is less hydrophobic. In some aspects, the different amino acid residue is an amino acid residue that is found at the same position in a genetic background of at least one species of SARS-CoV. In some aspects, the different amino acid residue is an amino acid residue that is found at the same position in a genetic background of at least one species of SARS-CoV and is less hydrophobic.

In some aspects, the compositions include a SARS-CoV-2 S glycoprotein variant, wherein the S glycoprotein variant comprises a RBD comprising a mutation of at least one amino acid residue in an ACE2 RBM relative to a wild-type RBD comprising the amino acid sequence of SEQ ID NO: 1, wherein the amino acid residue is L122 of SEQ ID NO: 1, and optionally F160 of SEQ ID NO: 1, and wherein the mutation is a substitution with a different amino acid residue. In some aspects, the RBD comprises a mutation of at least one amino acid residue in the ACE2 RBM relative to a wild-type RBD comprising the amino acid sequence of SEQ ID NO: 1, wherein the amino acid residue is L122 and F160 of SEQ ID NO: 1, and wherein the mutation is a substitution with a different amino acid residue. In some aspects, the S glycoprotein variant comprises a mutation of at least one additional amino acid residue in a first and/or second aggregation-prone region relative to the wild-type RBD, wherein the first aggregation-prone region comprises amino acid residues 122-126 of SEQ ID NO: 1, and the second aggregation-prone region comprises amino acid residues 158-162 of SEQ ID NO: 1, and wherein the mutation is a substitution with a different amino acid residue. In some aspects, the different amino acid residue is less hydrophobic. In some aspects, the different amino acid residue is an amino acid residue that is found at the same position in a genetic background of at least one species of SARS-CoV. In some aspects, the different amino acid residue is an amino acid residue that is found at the same position in a genetic background of at least one species of SARS-CoV and is less hydrophobic. In some aspects, the mutation of L122 of SEQ ID NO: 1 is a substitution of leucine with lysine (L122K), phenylalanine (L122F), tyrosine (L122Y), or serine (L122S). In some aspects, the mutation of F160 of SEQ ID NO: 1 is a substitution of phenylalanine with tryptophan (F160W), arginine (F160R), tyrosine (F160Y), or asparagine (F160N). In some aspects, the S glycoprotein variant comprises a mutation of at least one additional amino acid residue in a first and/or second aggregation-prone region relative to a wild-type RBD comprising the amino acid sequence of SEQ ID NO: 1, wherein the first aggregation-prone region comprises amino acid residues 122-126 of SEQ ID NO: 1, and the second aggregation-prone region comprises amino acid residues 158-162 of SEQ ID NO: 1, and wherein the mutation is a substitution with a different amino acid residue. In some aspects, the different amino acid residue is less hydrophobic, found at the same position in a genetic background of at least one species of SARS-CoV, or both.

In any of the foregoing or related aspects, the substitution is selected from the group: L122K, L122F, L122Y, L122S, L125Y, L125S, L125W, L125N, F126L, F126H, F126V, F126K, Y159V, Y159A, F160W, F160R, F160Y, F160N, F160M, and any combination thereof. In some aspects, the S glycoprotein variant comprises L122K. In some aspects, the S glycoprotein variant comprises L122K and F160W. In some aspects, the S glycoprotein variant comprises L122K, L125Y, F126L and F160W.

In any of the foregoing or related aspects, the RBD comprises a mutation of at least one asparagine-linked glycosylation site relative to the wild-type RBD. In some aspects, the mutation is selected from: (i) a substitution or deletion of the asparagine-linked glycosylation site at amino acid residue 1 of SEQ ID NO: 1; (ii) a substitution or deletion of the asparagine-linked glycosylation site at amino acid residue 13 of SEQ ID NO: 1; or (iii) a combination of (i)-(ii). In some aspects, the mutation is selected from: (i) a deletion of the asparagine-linked glycosylation site at amino acid residue 1 of SEQ ID NO: 1; and (ii) a substitution of the asparagine-linked glycosylation site at amino acid residue 13 of SEQ ID NO: 1. In some aspects, the RBD comprises a deletion of the asparagine-linked glycosylation site at amino acid residue 1 of SEQ ID NO: 1. In some aspects, the RBD comprises a substitution of the asparagine-linked glycosylation site at amino acid residue 13 of SEQ ID NO: 1. In some aspects, the substitution of the asparagine-linked glycosylation site is N to Q.

In some aspects, the compositions include a SARS-CoV-2 S glycoprotein variant, wherein the S glycoprotein variant comprises an amino acid sequence selected from: SEQ ID NO: 8, 9, 11, 15, and 16. In some aspects, the SARS-CoV-2 S glycoprotein variant, wherein the S glycoprotein variant comprises the amino acid sequence of SEQ ID NO: 8. In some aspects, the SARS-CoV-2 S glycoprotein variant, wherein the S glycoprotein variant comprises the amino acid sequence of SEQ ID NO: 9. In some aspects, the SARS-CoV-2 S glycoprotein variant, wherein the S glycoprotein variant comprises the amino acid sequence of SEQ ID NO: 11. In some aspects, the S glycoprotein variant comprises a mutation of at least one additional amino acid residue in a first and/or second aggregation-prone region relative to a wild-type RBD comprising the amino acid sequence of SEQ ID NO: 1, wherein the first aggregation-prone region comprises amino acid residues 122-126 of SEQ ID NO: 1, and the second aggregation-prone region comprises amino acid residues 158-162 of SEQ ID NO: 1, and wherein the mutation is a substitution with a different amino acid residue. In some aspects, the different amino acid residue is less hydrophobic, found at the same position in a genetic background of at least one species of SARS-CoV, or both.

In any of the foregoing or related aspects the SARS-CoV-2 S glycoprotein variant comprises a mutation of at least one additional amino acid residue in a first and/or second aggregation-prone region that is not part of the ACE2 RBM, wherein the first aggregation-prone region comprises amino acid residues 36-40 of SEQ ID NO: 1, and the second aggregation-prone region comprises amino acid residues 185-189 of SEQ ID NO: 1, wherein the mutation is a substitution with a different amino acid residue. In some aspects, the different amino acid residue is less hydrophobic. In some aspects, the different amino acid residue is an amino acid residue that is found at the same position in a genetic background of at least one species of SARS-CoV. In some aspects, the different amino acid residue is an amino acid residue that is found at the same position in a genetic background of at least one species of SARS-CoV and is less hydrophobic. In some aspects, the at least one amino acid residue is selected from: V37, L38, L187, L188, and a combination thereof. In some aspects, the mutation of at least one additional amino acid residue is a substitution selected from: V37F, L38A, L38M, L38F, L187A, L187I, L188A, L188M, L188D, L188T, and a combination thereof.

In some aspects, the compositions include a SARS-CoV-2 S glycoprotein variant comprising an amino acid sequence selected from the group consisting of SEQ ID NOs: 6, 7, 12-14, and 18.

In any of the foregoing or related aspects, the SARS-CoV-2 S glycoprotein variant comprises at least one additional amino acid residue substitution selected from: P7D, V11, A18P, N24E, R27K, Y35W, V37F, K48R, S53D, L60Y, F62W, I72V, R78D, Q84A, K87V, D98N, L111I, L125N, Q168D, Y178H, L188D, V194R, and any combination thereof. In some aspects, the SARS-CoV-2 S glycoprotein variant comprises an amino acid sequence selected from any one of SEQ ID NOs: 26-47.

In some aspects, the SARS-CoV-2 S glycoprotein variant described herein has reduced hydrophobicity relative to a SARS-CoV-2 S glycoprotein not having the at least one mutation. In some aspects, the hydrophobicity is reduced by at least 1.1 fold, 1.2 fold, 1.3 fold, 1.4 fold, 1.5 fold, 1.6 fold, 1.7 fold, 1.8 fold, 1.9 fold or 2.0 fold relative to the SARS-CoV-2 S glycoprotein not having the at least one mutation. In some aspects, the SARS-CoV-2 S glycoprotein described herein has reduced aggregation relative to an S glycoprotein not having the at least one mutation. In some aspects, the SARS-CoV-2 S glycoprotein described herein has increased thermostability relative to a SARS-CoV-2 S glycoprotein or fragment not having the at least one mutation. In some aspects, the SARS-CoV-2 S glycoprotein has disorder increased by about 5-30% within the ACE2 RBM relative to an SARS-CoV-2 S glycoprotein variant not having the at least one mutation. In some aspects, the SARS-CoV-2 S glycoprotein variant has increased immunogenicity relative to an SARS-CoV-2 S glycoprotein variant not having the at least one mutation. In some aspects, immunogenicity is measured by the level of IgG neutralizing antibodies produced. In some aspects, the SARS-CoV-2 S glycoprotein variant binds human ACE2 with substantially equivalent binding affinity to a SARS-CoV-2 S glycoprotein comprising the wild-type RBD. In some aspects, the SARS-CoV-2 S glycoprotein variant has increased binding affinity for human ACE2 relative to a SARS-CoV-2 S glycoprotein comprising the wild-type RBD.

In any of the foregoing or related aspects, the SARS-CoV-2 S glycoprotein variant described herein comprises a full-length RBD or portion thereof (e.g., receptor binding portion). In any of the foregoing or related aspects, the SARS-CoV-2 S glycoprotein variant described herein comprises an N-terminal domain or portion thereof. In some aspects, the S glycoprotein variant comprises an S2 subunit or portion thereof.

In some aspects, the disclosure provides a nucleic acid comprising a nucleotide sequence encoding the SARS-CoV-2 S glycoprotein variant described herein and the at least one linker comprising 2-12 phosphoserine residues. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In some aspects, the disclosure provides an expression vector comprising the nucleic acid. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In some aspects, the disclosure provides a cell comprising the expression vector or nucleic acid. The cell may be prokaryotic or eukaryotic. In some aspects, the cell is a yeast cell. In some aspects, the cell is a fungal cell. The cells can be transiently or stably engineered to incorporate the nucleic acids or expression vector of the disclosure, using techniques including but not limited to transformations, calcium phosphate co-precipitation, electroporation, or liposome mediated-, DEAE dextran mediated-, polycationic mediated-, or viral mediated transfection.

In some aspects, the disclosure provides a method for producing an SARS-CoV-2 S glycoprotein variant and the at least one linker comprising 2-12 phosphoserine residues, the method comprising maintaining a cell described herein under conditions permitting expression of the SARS-CoV-2 S glycoprotein variant. In some aspects, the expression of the SARS-CoV-2 S glycoprotein variant is increased relative to expression of an SARS-CoV-2 S glycoprotein variant not having the at least one mutation. In some aspects, aggregation of the SARS-CoV-2 S glycoprotein variant is reduced relative to aggregation of an SARS-CoV-2 S glycoprotein variant not having the at least one mutation, and wherein the reduced aggregation results in increased yield of the SARS-CoV-2 S glycoprotein variant.

The composition may comprise a plurality of identical SARS-CoV-2 S glycoprotein variants and the at least one linker comprising 2-12 phosphoserine residues, or may comprise 2, 3, or more different SARS-CoV-2 S glycoprotein variants and the at least one linker comprising 2-12 phosphoserine residues.

TABLE3
Sequences

| SEQ | | |
|---|---|---|
| ID | | |
| NO | Name | Sequence |
| 1 | RBD-A | NITNLCPFGEVFNATRFASVYAWNRKRISNCVADYSVLYNS |
| | (Native | ASFSTFKCYGVSPTKLNDLCFTNVYADSFVIRGDEVRQIAP |
| | SARS-CoV- | GQTGKIADYNYKLPDDFTGCVIAWNSNNLDSKVGGNYNYLY |
| | 2 | RLFRKSNLKPFERDISTEIYQAGSTPCNGVEGFNCYFPLQS |
| | Sequence) | YGFQPTNGVGYQPYRVVVLSFELLHAPATVCGPKKSTN |
| 2 | RBD-B | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | (Native | QTGKIADYNYKLPDDFTGCVIAWNSNNLDSKVGGNYNYLYR |
| | Sequence | LFRKSNLKPFERDISTEIYQAGSTPCNGVEGFNCYFPLQSY |
| | minus N1) | GFQPTNGVGYQPYRVVVLSFELLHAPATVCGPKKSTN |
| 3 | RBD-C | NITNLCPFGEVFQATRFASVYAWNRKRISNCVADYSVLYNS |
| | N13Q | ASFSTFKCYGVSPTKLNDLCFTNVYADSFVIRGDEVRQIAP |
| | | GQTGKIADYNYKLPDDFTGCVIAWNSNNLDSKVGGNYNYLY |
| | | RLFRKSNLKPFERDISTEIYQAGSTPCNGVEGFNCYFPLQS |
| | | YGFQPTNGVGYQPYRVVVLSFELLHAPATVCGPKKSTN |
| 4 | RBD-D | ITNLCPFGEVFQATRFASVYAWNRKRISNCVADYSVLYNSA |
| | N1del | SFSTFKCYGVSPTKLNDLCFTNVYADSFVIRGDEVRQIAPG |
| | N13Q | QTGKIADYNYKLPDDFTGCVIAWNSNNLDSKVGGNYNYLYR |
| | | LFRKSNLKPFERDISTEIYQAGSTPCNGVEGFNCYFPLQSY |
| | | GFQPTNGVGYQPYRVVVLSFELLHAPATVCGPKKSTN |
| 5 | RBD-E | ITNLCPFGEVFNATKFPSVYAWERKKISNCVADYSVLYNST |
| | N1del 230 | FFSTFKCYGVSATKLNDLCFSNVYADSFVVKGDDVRQIAPG |
| | Histag | QTGVIADYNYKLPDDFMGCVLAWNTRNIDATSTGNYNYKYR |
| | | YLRHGKLRPFERDISNVPFSPDGKPCTPPALNCYWPLNDYG |
| | | FYTTTGIGYQPYRVVVLSFELLNAPATVCGPKLSTDLIKNQ |
| | | CVNFNFNGLTGTGGSLEVLFQGPGSHHHHHHHHHH |
| 6 | RBD-G | ITNLCPFGEVFNATRFASVYAWNRKRISNCVADYSVAYNSA |
| | N1del | SFSTFKCYGVSPTKLNDLCFTNVYADSFVIRGDEVRQIAPG |
| | L38A | QTGKIADYNYKLPDDFTGCVIAWNSNNLDSKVGGNYNYKYR |
| | L122K | YLRKSNLKPFERDISTEIYQAGSTPCNGVEGFNCYWPLQSY |
| | L125Y | GFQPTNGVGYQPYRVVVLSFEAAHAPATVCGPKKSTN |
| | F126L | |
| | F160W | |
| | L187A | |
| | L188A | |
| 7 | RBD-H | ITNLCPFGEVFNATRFASVYAWNRKRISNCVADYSVAYNSA |
| | N1del | SFSTFKCYGVSPTKLNDLCFTNVYADSFVIRGDEVRQIAPG |
| | L38A | QTGKIADYNYKLPDDFTGCVIAWNSNNLDSKVGGNYNYKYR |
| | L122K | LFRKSNLKPFERDISTEIYQAGSTPCNGVEGFNCYWPLQSY |
| | F160W | GFQPTNGVGYQPYRVVVLSFEAAHAPATVCGPKKSTN |
| | L187A | |
| | L188A | |
| 8 | RBD-I | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | L125Y | YLRKSNLKPFERDISTEIYQAGSTPCNGVEGFNCYWPLQSY |
| | F160W | GFQPTNGVGYQPYRVVVLSFELLHAPATVCGPKKSTN |
| | F126L | |
| 9 | RBD-J | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | F160W | LFRKSNLKPFERDISTEIYQAGSTPCNGVEGFNCYWPLQSY |
| | | GFQPTNGVGYQPYRVVVLSFELLHAPATVCGPKKSTN |
| 10 | RBD-K | ITNLCPFGEVFNATRFASVYAWNRKRISNCVADYSVAYNSA |
| | N1del | SFSTFKCYGVSPTKLNDLCFTNVYADSFVIRGDEVRQIAPG |
| | L38A | QTGKIADYNYKLPDDFTGCVIAWNSNNLDSKVGGNYNYLYR |
| | L187A | LFRKSNLKPFERDISTEIYQAGSTPCNGVEGFNCYFPLQSY |
| | L188A | GFQPTNGVGYQPYRVVVLSFEAAHAPATVCGPKKSTN |
| 11 | RBD-L | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | | LFRKSNLKPFERDISTEIYQAGSTPCNGVEGFNCYFPLQSY |
| | | GFQPTNGVGYQPYRVVVLSFELLHAPATVCGPKKSTN |
| 12 | RBD-M | ITNLCPFGEVFNATRFASVYAWNRKRISNCVADYSVMYNSA |
| | N1del | SFSTFKCYGVSPTKLNDLCFTNVYADSFVIRGDEVRQIAPG |
| | L38M | QTGKIADYNYKLPDDFTGCVIAWNSNNLDSKVGGNYNYLYR |
| | | LFRKSNLKPFERDISTEIYQAGSTPCNGVEGFNCYFPLQSY |
| | | GFQPTNGVGYQPYRVVVLSFELLHAPATVCGPKKSTN |
| 13 | RBD-N | ITNLCPFGEVFNATRFASVYAWNRKRISNCVADYSVMYNSA |
| | N1del | SFSTFKCYGVSPTKLNDLCFTNVYADSFVIRGDEVRQIAPG |
| | L38M | QTGKIADYNYKLPDDFTGCVIAWNSNNLDSKVGGNYNYLYR |
| | L188M | LFRKSNLKPFERDISTEIYQAGSTPCNGVEGFNCYFPLQSY |
| | | GFQPTNGVGYQPYRVVVLSFELMHAPATVCGPKKSTN |
| 14 | RBD-O | ITNLCPFGEVFNATRFASVYAWNRKRISNCVADYSVMYNSA |
| | N1del | SFSTFKCYGVSPTKLNDLCFTNVYADSFVIRGDEVRQIAPG |
| | L38M | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | L188M | GFQPTNGVGYQPYRVVVLSFELMHAPATVCGPKKSTN |
| 15 | RBD-P | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | Y159R | QTGKIADYNYKLPDDFTGCVIAWNSNNLDSKVGGNYNYLYR |
| | | LFRKSNLKPFERDISTEIYQAGSTPCNGVEGFNCRFPLQSY |
| | | GFQPTNGVGYQPYRVVVLSFELLHAPATVCGPKKSTN |
| 16 | RBD-Q | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | F160M | QTGKIADYNYKLPDDFTGCVIAWNSNNLDSKVGGNYNYLYR |
| | | LFRKSNLKPFERDISTEIYQAGSTPCNGVEGFNCYMPLQSY |
| | | GFQPTNGVGYQPYRVVVLSFELLHAPATVCGPKKSTN |
| 17 | RBD-R | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | Y159R | QTGKIADYNYKLPDDFTGCVIAWNSNNLDSKVGGNYNYLYR |
| | F160M | LFRKSNLKPFERDISTEIYQAGSTPCNGVEGFNCRMPLQSY |
| | | GFQPTNGVGYQPYRVVVLSFELLHAPATVCGPKKSTN |
| 18 | RBD-S | ITNLCPFGEVFNATRFASVYAWNRKRISNCVADYSVMYNSA |
| | N1del | SFSTFKCYGVSPTKLNDLCFTNVYADSFVIRGDEVRQIAPG |
| | L38M | QTGKIADYNYKLPDDFTGCVIAWNSNNLDSKVGGNYNYKYR |
| | L122K | LFRKSNLKPFERDISTEIYQAGSTPCNGVEGFNCRMPLQSY |
| | Y159R | GFQPTNGVGYQPYRVVVLSFELMHAPATVCGPKKSTN |
| | F160M | |
| | L188M | |
| 19 | RBD-T | ITNLCPFGEVFNATRFASVYAWNRKRISNCVADYSVLYNSA |
| | L125Y | SFSTFKCYGVSPTKLNDLCFTNVYADSFVIRGDEVRQIAPG |
| | F126L | QTGKIADYNYKLPDDFTGCVIAWNSNNLDSKVGGNYNYLYR |
| | | YLRKSNLKPFERDISTEIYQAGSTPCNGVEGFNCYFPLQSY |
| | | GFQPTNGVGYQPYRVVVLSFELLHAPATVCGPKKSTN |
| 20 | RBD-U | ITNLCPFGEVFNATRFASVYAWNRKRISNCVADYSVLYNSA |
| | L122K | SFSTFKCYGVSPTKLNDLCFTNVYADSFVIRGDEVRQIAPG |
| | L125Y | QTGKIADYNYKLPDDFTGCVIAWNSNNLDSKVGGNYNYKYR |
| | F126L | YLRKSNLKPFERDISTEIYQAGSTPCNGVEGFNCYFPLQSY |
| | | GFQPTNGVGYQPYRVVVLSFELLHAPATVCGPKKSTN |
| 21 | RBD-V | ITNLCPFGEVFNATRFASVYAWNRKRISNCVADYSVLYNSA |
| | L125S | SFSTFKCYGVSPTKLNDLCFTNVYADSFVIRGDEVRQIAPG |
| | F126L | QTGKIADYNYKLPDDFTGCVIAWNSNNLDSKVGGNYNYLYR |
| | | SLRKSNLKPFERDISTEIYQAGSTPCNGVEGFNCYFPLQSY |
| | | GFQPTNGVGYQPYRVVVLSFELLHAPATVCGPKKSTN |
| 22 | RBD-W | ITNLCPFGEVFNATRFASVYAWNRKRISNCVADYSVLYNSA |
| | L122K | SFSTFKCYGVSPTKLNDLCFTNVYADSFVIRGDEVRQIAPG |
| | L125S | QTGKIADYNYKLPDDFTGCVIAWNSNNLDSKVGGNYNYKYR |
| | F126L | SLRKSNLKPFERDISTEIYQAGSTPCNGVEGFNCYFPLQSY |
| | | GFQPTNGVGYQPYRVVVLSFELLHAPATVCGPKKSTN |
| 23 | RBD-X | ITNLCPFGEVFNATRFASVYAWNRKRISNCVADYSVLYNSA |
| | L122A | SFSTFKCYGVSPTKLNDLCFTNVYADSFVIRGDEVRQIAPG |
| | | QTGKIADYNYKLPDDFTGCVIAWNSNNLDSKVGGNYNYAYR |
| | | LFRKSNLKPFERDISTEIYQAGSTPCNGVEGFNCYFPLQSY |
| | | GFQPTNGVGYQPYRVVVLSFELLHAPATVCGPKKSTN |
| 24 | RBD-Y | ITNLCPFGEVFNATRFASVYAWNRKRISNCVADYSVLYNSA |
| | L122K | SFSTFKCYGVSPTKLNDLCFTNVYADSFVIRGDEVRQIAPG |
| | Y159V | QTGKIADYNYKLPDDFTGCVIAWNSNNLDSKVGGNYNYKYR |
| | F160R | LFRKSNLKPFERDISTEIYQAGSTPCNGVEGFNCVRPLQSY |
| | | GFQPTNGVGYQPYRVVVLSFELLHAPATVCGPKKSTN |
| 25 | RBD-Z | ITNLCPFGEVFNATRFASVYAWNRKRISNCVADYSVLYNSA |
| | L122K | SFSTFKCYGVSPTKLNDLCFTNVYADSFVIRGDEVRQIAPG |
| | F160N | QTGKIADYNYKLPDDFTGCVIAWNSNNLDSKVGGNYNYKYR |
| | | LFRKSNLKPFERDISTEIYQAGSTPCNGVEGFNCYNPLQSY |
| | | GFQPTNGVGYQPYRVVVLSFELLHAPATVCGPKKSTN |
| 26 | RBD-Lmut1 | ITNLCPFGEVFNATRFASVYAWERKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | N24E | LFRKSNLKPFERDISTEIYQAGSTPCNGVEGFNCYFPLQSY |
| | | GFQPTNGVGYQPYRVVVLSFELLHAPATVCGPKKSTN |
| 27 | RBD-Lmut2 | ITNLCPFGEVFNATRFASVYAWNRKRISNCVADWSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | Y35W | LFRKSNLKPFERDISTEIYQAGSTPCNGVEGFNCYFPLQSY |
| | | GFQPTNGVGYQPYRVVVLSFELLHAPATVCGPKKSTN |
| 28 | RBD-Lmut3 | ITNLCPFGEVFNATRFASVYAWNRKRISNCVADYSFLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | V37F | LFRKSNLKPFERDISTEIYQAGSTPCNGVEGFNCYFPLQSY |
| | | GFQPTNGVGYQPYRVVVLSFELLHAPATVCGPKKSTN |
| 29 | RBD-Lmut4 | ITNLCPFGEAFNATRFASVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | V11I | LFRKSNLKPFERDISTEIYQAGSTPCNGVEGFNCYFPLQSY |
| | | GFQPTNGVGYQPYRVVVLSFELLHAPATVCGPKKSTN |
| 30 | RBD-Lmut5 | ITNLCPFGEVFNATRFPSVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | A18P | LFRKSNLKPFERDISTEIYQAGSTPCNGVEGFNCYFPLQSY |
| | | GFQPTNGVGYQPYRVVVLSFELLHAPATVCGPKKSTN |
| 31 | RBD-Lmut6 | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | L122K | QTGVIADYNYKLPDDFTGCVIAWNSNNLDSKVGGNYNYKYR |
| | K87V | LFRKSNLKPFERDISTEIYQAGSTPCNGVEGFNCYFPLQSY |
| | | GFQPTNGVGYQPYRVVVLSFELLHAPATVCGPKKSTN |
| 32 | RBD-Lmut7 | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | L188D | LFRKSNLKPFERDISTEIYQAGSTPCNGVEGFNCYFPLQSY |
| | | GFQPTNGVGYQPYRVVVLSFELDHAPATVCGPKKSTN |
| 33 | RBD-Lmut8 | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | V194R | LFRKSNLKPFERDISTEIYQAGSTPCNGVEGFNCYFPLQSY |
| | | GFQPTNGVGYQPYRVVVLSFELLHAPATRCGPKKSTN |
| 34 | RBD-Lmut9 | ITNLCDFGEVFNATRFASVYAWNRKRISNCVADYSVLYNSA |
| | N1del | SFSTFKCYGVSPTKLNDLCFTNVYADSFVIRGDEVRQIAPG |
| | L122K P7D | QTGKIADYNYKLPDDFTGCVIAWNSNNLDSKVGGNYNYKYR |
| | | LFRKSNLKPFERDISTEIYQAGSTPCNGVEGFNCYFPLQSY |
| | | GFQPTNGVGYQPYRVVVLSFELLHAPATVCGPKKSTN |
| 35 | RBD- | ITNLCPFGEVFNATRFASVYAWNRKKISNCVADYSVLYNSA |
| | Lmut10 | SFSTFKCYGVSPTKLNDLCFTNVYADSFVIRGDEVRQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | R27K | GFQPTNGVGYQPYRVVVLSFELLHAPATVCGPKKSTN |
| 36 | RBD- | ITNLCPFGEVFNATRFASVYAWNRKRISNCVADYSVLYNSA |
| | Lmut11 | SFSTFKCYGVDPTKLNDLCFTNVYADSFVIRGDEVRQIAPG |
| | N1del | QTGKIADYNYKLPDDFTGCVIAWNSNNLDSKVGGNYNYKYR |
| | L122K 53D | LFRKSNLKPFERDISTEIYQAGSTPCNGVEGFNCYFPLQSY |
| | | GFQPTNGVGYQPYRVVVLSFELLHAPATVCGPKKSTN |
| 37 | RBD- | ITNLCPFGEVFNATRFASVYAWNRKRISNCVADYSVLYNSA |
| | Lmut12 | SFSTFKCYGVSPTKLNDYCFTNVYADSFVIRGDEVRQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | L60Y | GFQPTNGVGYQPYRVVVLSFELLHAPATVCGPKKSTN |
| 38 | RBD- | ITNLCPFGEVFNATRFASVYAWNRKRISNCVADYSVLYNSA |
| | Lmut13 | SFSTFKCYGVSPTKLNDLCWTNVYADSFVIRGDEVRQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | F62W | GFQPTNGVGYQPYRVVVLSFELLHAPATVCGPKKSTN |
| 39 | RBD- | ITNLCPFGEVFNATRFASVYAWNRKRISNCVADYSVLYNSA |
| | Lmut14 | SFSTFKCYGVSPTKLNDLCFTNVYADSFVIRGDEVDQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | R78D | GFQPTNGVGYQPYRVVVLSFELLHAPATVCGPKKSTN |
| 40 | RBD- | ITNLCPFGEVFNATRFASVYAWNRKRISNCVADYSVLYNSA |
| | Lmut15 | SFSTFKCYGVSPTKLNDLCFTNVYADSFVIRGDEVRQIAPG |
| | N1del | ATGKIADYNYKLPDDFTGCVIAWNSNNLDSKVGGNYNYKYR |
| | L122K | LFRKSNLKPFERDISTEIYQAGSTPCNGVEGFNCYFPLQSY |
| | Q84A | GFQPTNGVGYQPYRVVVLSFELLHAPATVCGPKKSTN |
| 41 | RBD- | ITNLCPFGEVFNATRFASVYAWNRKRISNCVADYSVLYNSA |
| | Lmut16 | SFSTFKCYGVSPTKLNDLCFTNVYADSFVIRGDEVRQIAPG |
| | N1del | QTGKIADYNYKLPDNFTGCVIAWNSNNLDSKVGGNYNYKYR |
| | L122K | LFRKSNLKPFERDISTEIYQAGSTPCNGVEGFNCYFPLQSY |
| | D98N | GFQPTNGVGYQPYRVVVLSFELLHAPATVCGPKKSTN |
| 42 | RBD- | ITNLCPFGEVFNATRFASVYAWNRKRISNCVADYSVLYNSA |
| | Lmut17 | SFSTFKCYGVSPTKLNDLCFTNVYADSFVIRGDEVRQIAPG |
| | N1del | QTGKIADYNYKLPDDFTGCVIAWNSNNIDSKVGGNYNYKYR |
| | L122K | LFRKSNLKPFERDISTEIYQAGSTPCNGVEGFNCYFPLQSY |
| | L111I | GFQPTNGVGYQPYRVVVLSFELLHAPATVCGPKKSTN |
| 43 | RBD- | ITNLCPFGEVFNATRFASVYAWNRKRISNCVADYSVLYNSA |
| | Lmut18 | SFSTFKCYGVSPTKLNDLCFTNVYADSFVIRGDEVRQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | Y178H | GFQPTNGVGYQPHRVVVLSFELLHAPATVCGPKKSTN |
| 44 | RBD- | ITNLCPFGEVFNATRFASVYAWNRKRISNCVADYSVLYNSA |
| | Lmut19 | SFSTFRCYGVSPTKLNDLCFTNVYADSFVIRGDEVRQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | K48R | GFQPTNGVGYQPYRVVVLSFELLHAPATVCGPKKSTN |
| 45 | RBD- | ITNLCPFGEVFNATRFASVYAWNRKRISNCVADYSVLYNSA |
| | Lmut20 | SFSTFKCYGVSPTKLNDLCFTNVYADSFVVRGDEVRQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLQSY |
| | I72V | GFQPTNGVGYQPYRVVVLSFELLHAPATVCGPKKSTN |
| 46 | RBD- | ITNLCPFGEVFNATRFASVYAWNRKRISNCVADYSVLYNSA |
| | Lmut21 | SFSTFKCYGVSPTKLNDLCFTNVYADSFVIRGDEVRQIAPG |
| | N1del | QTGKIADYNYKLPDDFTGCVIAWNSNNLDSKVGGNYNYKYR |
| | L122K | LFRKSNLKPFERDISTEIYQAGSTPCNGVEGFNCYFPLDSY |
| | Q168D | GFQPTNGVGYQPYRVVVLSFELLHAPATVCGPKKSTN |
| 47 | RBD- | ITNLCPFGEVFNATRFASVYAWNRKRISNCVADYSVLYNSA |
| | Lmut22 | SFSTFKCYGVSPTKLNDLCFTNVYADSFVIRGDEVRQIAPG |
| | N1del | QTGKIADYNYKLPDDFTGCVIAWNSNNLDSKVGGNYNYKYR |
| | L122K | NFRKSNLKPFERDISTEIYQAGSTPCNGVEGFNCYFPLQSY |
| | L125N | GFQPTNGVGYQPYRVVVLSFELLHAPATVCGPKKSTN |
| 48 | RBD-B | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | Histag | QTGKIADYNYKLPDDFTGCVIAWNSNNLDSKVGGNYNYLYR |
| | FreeCys | LFRKSNLKPFERDISTEIYQAGSTPCNGVEGFNCYFPLQSY |
| | | GFQPTNGVGYQPYRVVVLSFELLHAPATVCGPKKSTNHHHH |
| | | HHC |
| 49 | RBD-B | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | Long | QTGKIADYNYKLPDDFTGCVIAWNSNNLDSKVGGNYNYLYR |
| | Linker | LFRKSNLKPFERDISTEIYQAGSTPCNGVEGFNCYFPLQSY |
| | SpyTag | GFQPTNGVGYQPYRVVVLSFELLHAPATVCGPKKSTNGGDG |
| | | GDGGDGGAHIVMVDAYKPTK |
| 50 | RBD-J | ITNLCPFGEVFNATRFASVYAWNRKRISNCVADYSVLYNSA |
| | N1del | SFSTFKCYGVSPTKLNDLCFTNVYADSFVIRGDEVRQIAPG |
| | L122K | QTGKIADYNYKLPDDFTGCVIAWNSNNLDSKVGGNYNYKYR |
| | F160W | LFRKSNLKPFERDISTEIYQAGSTPCNGVEGFNCYWPLQSY |
| | Long | GFQPTNGVGYQPYRVVVLSFELLHAPATVCGPKKSTNGGDG |
| | Linker | GDGGDGGAHIVMVDAYKPTK |
| | SpyTag | |
| 51 | RBD-B | ITNLCPFGEVFNATRFASVYAWNRKRISNCVADYSVLYNSA |
| | 207C | SFSTFKCYGVSPTKLNDLCFTNVYADSFVIRGDEVRQIAPG |
| | | QTGKIADYNYKLPDDFTGCVIAWNSNNLDSKVGGNYNYLYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KC |
| 52 | RBD-J | ITNLCPFGEVFNATRFASVYAWNRKRISNCVADYSVLYNSA |
| | 207C | SFSTFKCYGVSPTKLNDLCFTNVYADSFVIRGDEVRQIAPG |
| | | QTGKIADYNYKLPDDFTGCVIAWNSNNLDSKVGGNYNYKYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KC |
| 53 | RBD-B | ITNLCPFGEVFNATRFASVYAWNRKRISNCVADYSVLYNSA |
| | C207S | SFSTFKCYGVSPTKLNDLCFTNVYADSFVIRGDEVRQIAPG |
| | 259C | QTGKIADYNYKLPDDFTGCVIAWNSNNLDSKVGGNYNYLYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KSVNFNFNGLTGTGVLTESNKKFLPFQQFGRDIADTTDAVR |
| | | DPQTLEILDITPC |
| 54 | RBD-B | ITNLCPFGEVFNATRFASVYAWNRKRISNCVADYSVLYNSA |
| | C207S | SFSTFKCYGVSPTKLNDLCFTNVYADSFVIRGDEVRQIAPG |
| | 259CPPC | QTGKIADYNYKLPDDFTGCVIAWNSNNLDSKVGGNYNYLYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KSVNFNFNGLTGTGVLTESNKKFLPFQQFGRDIADTTDAVR |
| | | DPQTLEILDITPCPPC |
| 55 | RBD-J | ITNLCPFGEVFNATRFASVYAWNRKRISNCVADYSVLYNSA |
| | C207S | SFSTFKCYGVSPTKLNDLCFTNVYADSFVIRGDEVRQIAPG |
| | 259C | QTGKIADYNYKLPDDFTGCVIAWNSNNLDSKVGGNYNYKYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KSVNFNFNGLTGTGVLTESNKKFLPFQQFGRDIADTTDAVR |
| | | DPQTLEILDITPC |
| 56 | RBD-J | ITNLCPFGEVFNATRFASVYAWNRKRISNCVADYSVLYNSA |
| | C207S | SFSTFKCYGVSPTKLNDLCFTNVYADSFVIRGDEVRQIAPG |
| | 259CPPC | QTGKIADYNYKLPDDFTGCVIAWNSNNLDSKVGGNYNYKYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KSVNFNFNGLTGTGVLTESNKKFLPFQQFGRDIADTTDAVR |
| | | DPQTLEILDITPCPPC |
| 57 | RBD-B 260 | ITNLCPFGEVFNATRFASVYAWNRKRISNCVADYSVLYNSA |
| | B-Long | SFSTFKCYGVSPTKLNDLCFTNVYADSFVIRGDEVRQIAPG |
| | | QTGKIADYNYKLPDDFTGCVIAWNSNNLDSKVGGNYNYLYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KCVNFNFNGLTGTGVLTESNKKFLPFQQFGRDIADTTDAVR |
| | | DPQTLEILDITPCS |
| 58 | RBD-J 260 | ITNLCPFGEVFNATRFASVYAWNRKRISNCVADYSVLYNSA |
| | J-Long | SFSTFKCYGVSPTKLNDLCFTNVYADSFVIRGDEVRQIAPG |
| | | QTGKIADYNYKLPDDFTGCVIAWNSNNLDSKVGGNYNYKYR |
| | | LFRKSNLKPFERDISTEIYQAGSTPCNGVEGFNCYWPLQSY |
| | | GFQPTNGVGYQPYRVVVLSFELLHAPATVCGPKKSTNLVKN |
| | | KCVNFNFNGLTGTGVLTESNKKFLPFQQFGRDIADTTDAVR |
| | | DPQTLEILDITPCS |
| 59 | SARS-CoV- | FVFLVLLPLVSSQCVNLTTRTQLPPAYTNSFTRGVYYPDKV |
| | 2 | FRSSVLHSTQDLFLPFFSNVTWFHAIHVSGTNGTKRFDNPV |
| | Spike | LPFNDGVYFASTEKSNIIRGWIFGTTLDSKTQSLLIVNNAT |
| | glyco- | NVVIKVCEFQFCNDPFLGVYYHKNNKSWMESEFRVYSSANN |
| | protein | CTFEYVSQPFLMDLEGKQGNFKNLREFVFKNIDGYFKIYSK |
| | Signal | HTPINLVRDLPQGFSALEPLVDLPIGINITRFQTLLALHRS |
| | peptide | YLTPGDSSSGWTAGAAAYYVGYLQPRTFLLKYNENGTITDA |
| | in | VDCALDPLSETKCTLKSFTVEKGIYQTSNFRVQPTESIVRF |
| | underline | PNITNLCPFGEVFNATRFASVYAWNRKRISNCVADYSVLYN |
| | RBD | SASFSTFKCYGVSPTKLNDLCFTNVYADSFVIRGDEVRQIA |
| | corre- | PGQTGKIADYNYKLPDDFTGCVIAWNSNNLDSKVGGNYNYL |
| | sponding to | YRLFRKSNLKPFERDISTEIYQAGSTPCNGVEGFNCYFPLQ |
| | SEQ ID | SYGFQPTNGVGYQPYRVVVLSFELLHAPATVCGPKKSTNLV |
| | NO: 1 in | KNKCVNFNFNGLTGTGVLTESNKKFLPFQQFGRDIADTTDA |
| | bold | VRDPQTLEILDITPCSFGGVSVITPGTNTSNQVAVLYQDVN |
| | | CTEVPVAIHADQLTPTWRVYSTGSNVFQTRAGCLIGAEHVN |
| | | NSYECDIPIGAGICASYQTQTNSPSGAGSVASQSIIAYTMS |
| | | LGAENSVAYSNNSIAIPTNFTISVTTEILPVSMTKTSVDCT |
| | | MYICGDSTECSNLLLQYGSFCTQLNRALTGIAVEQDKNTQE |
| | | VFAQVKQIYKTPPIKDFGGFNFSQILPDPSKPSKRSFIEDL |
| | | LFNKVTLADAGFIKQYGDCLGDIAARDLICAQKFNGLTVLP |
| | | PLLTDEMIAQYTSALLAGTITSGWTFGAGAALQIPFAMQMA |
| | | YRFNGIGVTQNVLYENQKLIANQFNSAIGKIQDSLSSTASA |
| | | LGKLQDVVNQNAQALNTLVKQLSSNFGAISSVLNDILSRLD |
| | | PPEAEVQIDRLITGRLQSLQTYVTQQLIRAAEIRASANLAA |
| | | TKMSECVLGQSKRVDFCGKGYHLMSFPQSAPHGVVFLHVTY |
| | | VPAQEKNFTTAPAICHDGKAHFPREGVFVSNGTHWFVTQRN |
| | | FYEPQIITTDNTFVSGNCDVVIGIVNNTVYDPLQPELDSFK |
| | | EELDKYFKNHTSPDVDLGDISGINASVVNIQKEIDRLNEVA |
| | | KNLNESLIDLQELGKYEQYIKWPWYIWLGFIAGLIAIVMVT |
| | | IMLCCMTSCCSCLKGCCSCGSCCKFDEDDSEPVLKGVKLHY |
| | | T |
| 60 | SARS-Cov- | FVFLVLLPLVSS |
| | 2 S | |
| | glyco- | |
| | protein | |
| | signal | |
| | peptide | |
| 61 | SARS-Cov- | QCVNLTTRTQLPPAYTNSFTRGVYYPDKVFRSSVLHSTQDL |
| | 2S | FLPFFSNVTWFHAIHVSGTNGTKRFDNPVLPFNDGVYFAST |
| | glyco- | EKSNIIRGWIFGTTLDSKTQSLLIVNNATNVVIKVCEFQFC |
| | protein | NDPFLGVYYHKNNKSWMESEFRVYSSANNCTFEYVSQPFLM |
| | without | DLEGKQGNFKNLREFVFKNIDGYFKIYSKHTPINLVRDLPQ |
| | signal | GFSALEPLVDLPIGINITRFQTLLALHRSYLTPGDSSSGWT |
| | peptide | AGAAAYYVGYLQPRTFLLKYNENGTITDAVDCALDPLSETK |
| | | CTLKSFTVEKGIYQTSNFRVQPTESIVRFPNITNLCPFGEV |
| | | FNATRFASVYAWNRKRISNCVADYSVLYNSASFSTFKCYGV |
| | | SPTKLNDLCFTNVYADSFVIRGDEVRQIAPGQTGKIADYNY |
| | | KLPDDFTGCVIAWNSNNLDSKVGGNYNYLYRLFRKSNLKPF |
| | | ERDISTEIYQAGSTPCNGVEGFNCYFPLQSYGFQPTNGVGY |
| | | QPYRVVVLSFELLHAPATVCGPKKSTNLVKNKCVNFNFNGL |
| | | TGTGVLTESNKKFLPFQQFGRDIADTTDAVRDPQTLEILDI |
| | | TPCSFGGVSVITPGTNTSNQVAVLYQDVNCTEVPVAIHADQ |
| | | LTPTWRVYSTGSNVFQTRAGCLIGAEHVNNSYECDIPIGAG |
| | | ICASYQTQTNSPSGAGSVASQSIIAYTMSLGAENSVAYSNN |
| | | SIAIPTNFTISVTTEILPVSMTKTSVDCTMYICGDSTECSN |
| | | LLLQYGSFCTQLNRALTGIAVEQDKNTQEVFAQVKQIYKTP |
| | | PIKDFGGFNFSQILPDPSKPSKRSFIEDLLFNKVTLADAGF |
| | | IKQYGDCLGDIAARDLICAQKFNGLTVLPPLLTDEMIAQYT |
| | | SALLAGTITSGWTFGAGAALQIPFAMQMAYRFNGIGVTQNV |
| | | LYENQKLIANQFNSAIGKIQDSLSSTASALGKLQDVVNQNA |
| | | QALNTLVKQLSSNFGAISSVLNDILSRLDPPEAEVQIDRLI |
| | | TGRLQSLQTYVTQQLIRAAEIRASANLAATKMSECVLGQSK |
| | | RVDFCGKGYHLMSFPQSAPHGVVFLHVTYVPAQEKNFTTAP |
| | | AICHDGKAHFPREGVFVSNGTHWFVTQRNFYEPQIITTDNT |
| | | FVSGNCDVVIGIVNNTVYDPLQPELDSFKEELDKYFKNHTS |
| | | PDVDLGDISGINASVVNIQKEIDRLNEVAKNLNESLIDLQE |
| | | LGKYEQYIKWPWYIWLGFIAGLIAIVMVTIMLCCMTSCCSC |
| | | LKGCCSCGSCCKFDEDDSEPVLKGVKLHYT |
| 62 | RBD-A | AACATCACCAACTTGTGCCCATTCGGTGAGGTTTTCAACGC |
| | (nucleotide | TACTAGATTCGCTTCCGTTTACGCCTGGAACAGAAAGAGAA |
| | sequence | TCTCCAACTGCGTTGCTGACTACTCCGTCTTGTACAACTCT |
| | of SEQ ID | GCTTCATTCTCCACCTTCAAGTGCTACGGTGTTTCCCCAAC |
| | NO: 1) | TAAGTTGAACGACCTGTGTTTCACTAACGTCTACGCCGACT |
| | | CCTTCGTTATTAGAGGTGACGAGGTTAGACAGATCGCTCCA |
| | | GGTCAAACTGGTAAGATCGCTGACTACAACTACAAGCTGCC |
| | | AGACGACTTCACCGGTTGTGTTATTGCTTGGAACTCCAACA |
| | | ACCTGGACTCCAAGGTTGGTGGTAACTACAATTACCTGTAC |
| | | CGTCTGTTCAGAAAGTCCAACCTGAAGCCATTCGAGAGAGA |
| | | CATCTCCACTGAGATCTACCAAGCTGGTTCCACTCCATGTA |
| | | ACGGTGTTGAGGGTTTCAACTGTTACTTCCCATTGCAGTCC |
| | | TACGGTTTCCAGCCAACTAATGGTGTTGGTTACCAGCCATA |
| | | CAGAGTCGTCGTTTTGTCCTTCGAGTTGTTGCATGCTCCAG |
| | | CTACTGTTTGCGGTCCAAAGAAGTCCACTAAC |
| 63 | RBD-B | ATCACCAACTTGTGTCCATTCGGTGAGGTTTTCAACGCTAC |
| | N1del | TAGATTCGCTTCCGTTTACGCCTGGAACAGAAAGAGAATCT |
| | (nucleotide | CCAACTGCGTTGCTGACTACTCCGTCTTGTACAACTCTGCT |
| | sequence | TCATTCTCCACCTTCAAGTGCTACGGTGTTTCCCCAACTAA |
| | of SEQ ID | GTTGAACGACCTGTGTTTCACTAACGTCTACGCCGACTCCT |
| | NO: 2) | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 64 | RBD-C | AACATCACCAACTTGTGCCCATTCGGTGAGGTTTTCCAGGC |
| | N13Q | TACTAGATTCGCTTCTGTTTACGCCTGGAACAGAAAGAGAA |
| | (nucleotide | TCTCCAACTGCGTTGCTGACTACTCCGTCTTGTACAACTCT |
| | sequence | GCTTCATTCTCCACCTTCAAGTGCTACGGTGTTTCCCCAAC |
| | of SEQ ID | TAAGTTGAACGACCTGTGTTTCACCAACGTTTACGCCGACT |
| | NO: 3) | CCTTCGTTATTAGAGGTGACGAGGTTAGACAGATCGCTCCA |
| | | GGTCAAACTGGTAAGATCGCTGACTACAACTACAAGCTGCC |
| | | AGACGACTTCACCGGTTGTGTTATTGCTTGGAACTCCAACA |
| | | ACCTGGACTCCAAGGTTGGTGGTAACTACAATTACCTGTAC |
| | | CGTCTGTTCAGAAAGTCCAACCTGAAGCCATTCGAGAGAGA |
| | | CATCTCCACTGAGATCTACCAAGCTGGTTCCACTCCATGTA |
| | | ACGGTGTTGAGGGTTTCAACTGCTACTTCCCATTGCAGTCT |
| | | TACGGTTTCCAGCCAACTAACGGTGTCGGTTACCAACCATA |
| | | CAGAGTTGTCGTTTTGTCCTTCGAGTTGTTGCACGCTCCAG |
| | | CTACTGTTTGTGGTCCAAAGAAGTCCACTAAC |
| 65 | RBD-D | ATCACCAACTTGTGTCCATTCGGTGAGGTTTTCCAGGCTAC |
| | N1del | TAGATTCGCTTCTGTTTACGCCTGGAACAGAAAGAGAATCT |
| | N13Q | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACCAACGTTTACGCCGACTCCT |
| | of SEQ ID | TCGTTATTAGAGGTGACGAGGTTAGACAGATCGCTCCAGGT |
| | NO: 4) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGCTACTTCCCATTGCAGTCTTAC |
| | | GGTTTCCAGCCAACTAACGGTGTCGGTTACCAACCATACAG |
| | | AGTTGTCGTTTTGTCCTTCGAGTTGTTGCACGCTCCAGCTA |
| | | CTGTTTGTGGTCCAAAGAAGTCCACTAAC |
| 66 | RBD-E | ATCACCAACTTGTGTCCATTCGGTGAGGTTTTCAACGCCAC |
| | N1del 230 | TAAGTTCCCATCTGTTTACGCCTGGGAGAGAAAGAAGATCT |
| | Histag | CCAACTGCGTTGCTGACTACTCCGTCTTGTACAACTCCACA |
| | (nucleotide | TTCTTCAGCACCTTCAAGTGCTACGGTGTTTCCGCTACTAA |
| | sequence | GTTGAACGACCTGTGTTTCTCCAACGTTTACGCCGACTCCT |
| | of SEQ ID | TCGTTGTTAAGGGTGACGACGTTAGACAGATTGCTCCAGGT |
| | NO: 5) | CAGACTGGTGTTATCGCTGACTACAACTACAAGCTGCCAGA |
| | | CGACTTCATGGGTTGTGTTTTGGCTTGGAACACCAGAAACA |
| | | TCGACGCTACTTCCACCGGTAATTACAATTACAAGTACCGT |
| | | TACCTGAGGCACGGTAAGCTTAGACCATTCGAGAGAGACAT |
| | | CTCTAACGTCCCATTCTCTCCAGACGGTAAGCCTTGTACTC |
| | | CTCCTGCTTTGAACTGTTACTGGCCATTGAACGACTACGGT |
| | | TTCTACACTACTACCGGTATCGGTTACCAGCCATACAGAGT |
| | | TGTTGTCTTGTCCTTCGAGTTGTTGAACGCTCCAGCTACTG |
| | | TTTGCGGTCCAAAGTTGTCCACCGACCTGATTAAGAACCAG |
| | | TGCGTCAACTTTAACTTCAACGGTCTGACTGGTACTGGTGG |
| | | TTCCTTGGAGGTTTTGTTTCAAGGTCCAGGTTCTCATCACC |
| | | ACCACCATCACCATCATCACCAC |
| 67 | RBD-G | ATCACCAACTTGTGTCCATTCGGTGAGGTTTTCAACGCTAC |
| | N1del | TAGATTCGCTTCCGTTTACGCCTGGAACAGAAAGAGAATCT |
| | L38A | CCAACTGCGTTGCTGACTACTCCGTCGCTTACAACTCTGCT |
| | L122K | TCATTCTCCACCTTCAAGTGCTACGGTGTTTCCCCAACTAA |
| | L125Y | GTTGAACGACCTGTGTTTCACTAACGTCTACGCCGACTCCT |
| | F126L | TCGTTATTAGAGGTGACGAGGTTAGACAGATCGCTCCAGGT |
| | F160W | CAAACTGGTAAGATCGCTGACTACAACTACAAGCTGCCAGA |
| | L187A | CGACTTCACCGGTTGTGTTATTGCTTGGAACTCCAACAACC |
| | L188A | TGGACTCCAAGGTTGGTGGTAACTACAATTACAAATACCGT |
| | (nucleotide | TATTTGAGAAAGTCCAACCTGAAGCCATTCGAGAGAGACAT |
| | sequence | CTCCACTGAGATCTACCAAGCTGGTTCCACTCCATGTAACG |
| | of SEQ ID | GTGTTGAGGGTTTCAACTGTTACTGGCCATTGCAGTCCTAC |
| | NO: 6) | GGTTTCCAGCCAACTAATGGTGTTGGTTACCAGCCATACAG |
| | | AGTCGTCGTTTTGTCCTTCGAGGCTGCTCATGCTCCAGCTA |
| | | CTGTTTGCGGTCCAAAGAAGTCCACTAAC |
| 68 | RBD-H | ATCACCAACTTGTGTCCATTCGGTGAGGTTTTCAACGCTAC |
| | N1del | TAGATTCGCTTCCGTTTACGCCTGGAACAGAAAGAGAATCT |
| | L38A | CCAACTGCGTTGCTGACTACTCCGTCGCTTACAACTCTGCT |
| | L122K | TCATTCTCCACCTTCAAGTGCTACGGTGTTTCCCCAACTAA |
| | F160W | GTTGAACGACCTGTGTTTCACTAACGTCTACGCCGACTCCT |
| | L187A | TCGTTATTAGAGGTGACGAGGTTAGACAGATCGCTCCAGGT |
| | L188A | CAAACTGGTAAGATCGCTGACTACAACTACAAGCTGCCAGA |
| | (nucleotide | CGACTTCACCGGTTGTGTTATTGCTTGGAACTCCAACAACC |
| | sequence | TGGACTCCAAGGTTGGTGGTAACTACAATTACAAATACCGT |
| | of SEQ ID | TTGTTTAGAAAGTCCAACCTGAAGCCATTCGAGAGAGACAT |
| | NO: 7) | CTCCACTGAGATCTACCAAGCTGGTTCCACTCCATGTAACG |
| | | GTGTTGAGGGTTTCAACTGTTACTGGCCATTGCAGTCCTAC |
| | | GGTTTCCAGCCAACTAATGGTGTTGGTTACCAGCCATACAG |
| | | AGTCGTCGTTTTGTCCTTCGAGGCTGCTCATGCTCCAGCTA |
| | | CTGTTTGCGGTCCAAAGAAGTCCACTAAC |
| 69 | RBD-1 | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | L125Y | TCATTCTCCACCTTCAAGTGCTACGGTGTTTCCCCAACTAA |
| | F160W | GTTGAACGACCTGTGTTTCACTAACGTCTACGCCGACTCCT |
| | F126L | TCGTTATTAGAGGTGACGAGGTTAGACAGATCGCTCCAGGT |
| | (nucleotide | CAAACTGGTAAGATCGCTGACTACAACTACAAGCTGCCAGA |
| | sequence | CGACTTCACCGGTTGTGTTATTGCTTGGAACTCCAACAACC |
| | of SEQ ID | TGGACTCCAAGGTTGGTGGTAACTACAATTACAAATACCGT |
| | NO: 8) | TATTTGAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTGGCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 70 | RBD-J | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | F160W | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 9) | CGACTTCACCGGTTGTGTTATTGCTTGGAACTCCAACAACC |
| | | TGGACTCCAAGGTTGGTGGTAACTACAATTACAAATACCGT |
| | | TTGTTTAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTGGCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 71 | RBD-K | ATCACCAACTTGTGTCCATTCGGTGAGGTTTTCAACGCTAC |
| | N1del | TAGATTCGCTTCCGTTTACGCCTGGAACAGAAAGAGAATCT |
| | L38A | CCAACTGCGTTGCTGACTACTCCGTCGCTTACAACTCTGCT |
| | L187A | TCATTCTCCACCTTCAAGTGCTACGGTGTTTCCCCAACTAA |
| | L188A | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 10) | TGGACTCCAAGGTTGGTGGTAACTACAATTACTTGTACCGT |
| | | TTGTTTAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTTCCATTGCAGTCCTAC |
| | | GGTTTCCAGCCAACTAATGGTGTTGGTTACCAGCCATACAG |
| | | AGTCGTCGTTTTGTCCTTCGAGGCTGCTCATGCTCCAGCTA |
| | | CTGTTTGCGGTCCAAAGAAGTCCACTAAC |
| 72 | RBD-L | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 11) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 73 | RBD-M | ATCACCAACTTGTGTCCATTCGGTGAGGTTTTCAACGCTAC |
| | N1del | TAGATTCGCTTCCGTTTACGCCTGGAACAGAAAGAGAATCT |
| | L38M | CCAACTGCGTTGCTGACTACTCCGTCATGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 12) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 74 | RBD-N | ATCACCAACTTGTGTCCATTCGGTGAGGTTTTCAACGCTAC |
| | N1del | TAGATTCGCTTCCGTTTACGCCTGGAACAGAAAGAGAATCT |
| | L38M | CCAACTGCGTTGCTGACTACTCCGTCATGTACAACTCTGCT |
| | L188M | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 13) | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGATGCATGCTCCAGCTA |
| | | CTGTTTGCGGTCCAAAGAAGTCCACTAAC |
| 75 | RBD-O | ATCACCAACTTGTGTCCATTCGGTGAGGTTTTCAACGCTAC |
| | N1del | TAGATTCGCTTCCGTTTACGCCTGGAACAGAAAGAGAATCT |
| | L38M | CCAACTGCGTTGCTGACTACTCCGTCATGTACAACTCTGCT |
| | L122K | TCATTCTCCACCTTCAAGTGCTACGGTGTTTCCCCAACTAA |
| | L188M | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 14) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGATGCATGCTCCAGCTA |
| | | CTGTTTGCGGTCCAAAGAAGTCCACTAAC |
| 76 | RBD-P | ATCACCAACTTGTGTCCATTCGGTGAGGTTTTCAACGCTAC |
| | N1del | TAGATTCGCTTCCGTTTACGCCTGGAACAGAAAGAGAATCT |
| | Y159R | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 15) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTAGATTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 77 | RBD-Q | ATCACCAACTTGTGTCCATTCGGTGAGGTTTTCAACGCTAC |
| | N1del | TAGATTCGCTTCCGTTTACGCCTGGAACAGAAAGAGAATCT |
| | F160M | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 16) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACATGCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 78 | RBD-R | ATCACCAACTTGTGTCCATTCGGTGAGGTTTTCAACGCTAC |
| | N1del | TAGATTCGCTTCCGTTTACGCCTGGAACAGAAAGAGAATCT |
| | Y159R | CCAACTGCGTTGCTGACTACTCCGTCTTGTACAACTCTGCT |
| | F160M | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 17) | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTAGAATGCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 79 | RBD-S | ATCACCAACTTGTGTCCATTCGGTGAGGTTTTCAACGCTAC |
| | N1del | TAGATTCGCTTCCGTTTACGCCTGGAACAGAAAGAGAATCT |
| | L38M | CCAACTGCGTTGCTGACTACTCCGTCATGTACAACTCTGCT |
| | L122K | TCATTCTCCACCTTCAAGTGCTACGGTGTTTCCCCAACTAA |
| | Y159R | GTTGAACGACCTGTGTTTCACTAACGTCTACGCCGACTCCT |
| | F160M | TCGTTATTAGAGGTGACGAGGTTAGACAGATCGCTCCAGGT |
| | L188M | CAAACTGGTAAGATCGCTGACTACAACTACAAGCTGCCAGA |
| | (nucleotide | CGACTTCACCGGTTGTGTTATTGCTTGGAACTCCAACAACC |
| | sequence | TGGACTCCAAGGTTGGTGGTAACTACAATTACAAATACCGT |
| | of SEQ ID | CTGTTCAGAAAGTCCAACCTGAAGCCATTCGAGAGAGACAT |
| | NO: 18) | CTCCACTGAGATCTACCAAGCTGGTTCCACTCCATGTAACG |
| | | GTGTTGAGGGTTTCAACTGTAGAATGCCATTGCAGTCCTAC |
| | | GGTTTCCAGCCAACTAATGGTGTTGGTTACCAGCCATACAG |
| | | AGTCGTCGTTTTGTCCTTCGAGTTGATGCATGCTCCAGCTA |
| | | CTGTTTGCGGTCCAAAGAAGTCCACTAAC |
| 80 | RBD-T | ATCACCAACTTGTGTCCATTCGGTGAGGTTTTCAACGCTAC |
| | L125Y | TAGATTCGCTTCCGTTTACGCCTGGAACAGAAAGAGAATCT |
| | F126L | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 19) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | TACCTGAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 81 | RBD-U | ATCACCAACTTGTGTCCATTCGGTGAGGTTTTCAACGCTAC |
| | L122K | TAGATTCGCTTCCGTTTACGCCTGGAACAGAAAGAGAATCT |
| | L125Y | CCAACTGCGTTGCTGACTACTCCGTCTTGTACAACTCTGCT |
| | F126L | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 20) | CGACTTCACCGGTTGTGTTATTGCTTGGAACTCCAACAACC |
| | | TGGACTCCAAGGTTGGTGGTAATTACAATTACAAGTACCGT |
| | | TACCTGAGGAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 82 | RBD-V | ATCACCAACTTGTGTCCATTCGGTGAGGTTTTCAACGCTAC |
| | L125S | TAGATTCGCTTCCGTTTACGCCTGGAACAGAAAGAGAATCT |
| | F126L | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 21) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACAGA |
| | | TCCCTGAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 83 | RBD-W | ATCACCAACTTGTGTCCATTCGGTGAGGTTTTCAACGCTAC |
| | L122K | TAGATTCGCTTCCGTTTACGCCTGGAACAGAAAGAGAATCT |
| | L125S | CCAACTGCGTTGCTGACTACTCCGTCTTGTACAACTCTGCT |
| | F126L | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 22) | CGACTTCACCGGTTGTGTTATTGCTTGGAACTCCAACAACC |
| | | TGGACTCCAAGGTTGGTGGTAATTACAATTACAAGTACAGG |
| | | TCCCTGAGGAAGTCCAACTTGAAGCCATTCGAGAGAGACAT |
| | | CTCCACCGAGATCTACCAAGCTGGTTCTACTCCATGTAACG |
| | | GTGTCGAGGGTTTCAACTGTTACTTCCCATTGCAGTCCTAC |
| | | GGTTTCCAGCCAACTAACGGTGTTGGTTACCAGCCATACAG |
| | | AGTTGTCGTTTTGTCCTTCGAGTTGTTGCACGCTCCAGCTA |
| | | CTGTTTGTGGTCCAAAGAAGTCCACTAAC |
| 84 | RBD-X | ATCACCAACTTGTGTCCATTCGGTGAGGTTTTCAACGCTAC |
| | L122A | TAGATTCGCTTCCGTTTACGCCTGGAACAGAAAGAGAATCT |
| | (nucleotide | CCAACTGCGTTGCTGACTACTCCGTCTTGTACAACTCTGCT |
| | sequence | TCATTCTCCACCTTCAAGTGCTACGGTGTTTCCCCAACTAA |
| | of SEQ ID | GTTGAACGACCTGTGTTTCACTAACGTCTACGCCGACTCCT |
| | NO: 23) | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACGCCTACAGG |
| | | CTGTTCAGAAAGTCCAACTTGAAGCCATTCGAGAGAGACAT |
| | | CTCCACCGAGATCTACCAAGCTGGTTCTACTCCATGTAACG |
| | | GTGTCGAGGGTTTCAACTGTTACTTCCCATTGCAGTCCTAC |
| | | GGTTTCCAGCCAACTAACGGTGTTGGTTACCAGCCATACAG |
| | | AGTTGTCGTTTTGTCCTTCGAGTTGTTGCACGCTCCAGCTA |
| | | CTGTTTGTGGTCCAAAGAAGTCCACTAAC |
| 85 | RBD-Y | ATCACCAACTTGTGTCCATTCGGTGAGGTTTTCAACGCTAC |
| | L122K | TAGATTCGCTTCCGTTTACGCCTGGAACAGAAAGAGAATCT |
| | Y159V | CCAACTGCGTTGCTGACTACTCCGTCTTGTACAACTCTGCT |
| | F160R | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 24) | CGACTTCACCGGTTGTGTTATTGCTTGGAACTCCAACAACC |
| | | TGGACTCCAAGGTTGGTGGTAATTACAATTACAAGTACAGG |
| | | CTGTTCCGTAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGCGTCAGACCATTGCAATCTTAC |
| | | GGTTTCCAGCCAACTAACGGTGTCGGTTACCAACCATACAG |
| | | AGTTGTCGTTTTGTCCTTCGAGTTGTTGCACGCTCCAGCTA |
| | | CTGTTTGTGGTCCAAAGAAGTCCACTAAC |
| 86 | RBD-Z | ATCACCAACTTGTGTCCATTCGGTGAGGTTTTCAACGCTAC |
| | L122K | TAGATTCGCTTCCGTTTACGCCTGGAACAGAAAGAGAATCT |
| | F160N | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 25) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAATTACAATTACAAGTACAGG |
| | | CTGTTCCGTAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACAACCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 87 | RBD-Lmut1 | ATCACCAACTTGTGTCCATTCGGTGAGGTTTTCAACGCTAC |
| | N1del | TAGATTCGCTTCCGTTTACGCCTGGGAAAGAAAGAGAATCT |
| | L122K | CCAACTGCGTTGCTGACTACTCCGTCTTGTACAACTCTGCT |
| | N24E | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 26) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 88 | RBD-Lmut2 | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTGGTCCGTCTTGTACAACTCTGCT |
| | Y35W | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 27) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 89 | RBD-Lmut3 | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCTTTTTGTACAACTCTGCT |
| | V37F | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 28) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 90 | RBD-Lmut4 | ATCACCAACTTGTGTCCATTCGGTGAGATTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | V11I | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 2 9 | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 91 | RBD-Lmut5 | ATCACCAACTTGTGTCCATTCGGTGAGGTTTTCAACGCTAC |
| | N1del | TAGATTCCCATCCGTTTACGCCTGGAACAGAAAGAGAATCT |
| | L122K | CCAACTGCGTTGCTGACTACTCCGTCTTGTACAACTCTGCT |
| | A18P | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 30) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 92 | RBD-Lmut6 | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | K87V | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTGTTATCGCTGACTACAACTACAAGCTGCCAGA |
| | NO: 31) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 93 | RBD-Lmut7 | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | L188D | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 32) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGGATCATGCTCCAGCTA |
| | | CTGTTTGCGGTCCAAAGAAGTCCACTAAC |
| 94 | RBD-Lmut8 | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | V194R | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 33) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTAGATGCGGTCCAAAGAAGTCCACTAAC |
| 95 | RBD-Lmut9 | ATCACCAACTTGTGTGATTTCGGTGAGGTTTTCAACGCTAC |
| | N1del | TAGATTCGCTTCCGTTTACGCCTGGAACAGAAAGAGAATCT |
| | L122K P7D | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 34) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 96 | RBD- | ATCACCAACTTGTGTCCATTCGGTGAGGTTTTCAACGCTAC |
| | Lmut10 | TAGATTCGCTTCCGTTTACGCCTGGAACAGAAAGAAAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | R27K | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 35) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 97 | RBD- | ATCACCAACTTGTGTCCATTCGGTGAGGTTTTCAACGCTAC |
| | Lmut11 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K 53D | TCATTCTCCACCTTCAAGTGCTACGGTGTTGATCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 37) | CGACTTCACCGGTTGTGTTATTGCTTGGAACTCCAACAACC |
| | | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 98 | RBD- | ATCACCAACTTGTGTCCATTCGGTGAGGTTTTCAACGCTAC |
| | Lmut12 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | L60Y | GTTGAACGACTATTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 38) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 99 | RBD- | ATCACCAACTTGTGTCCATTCGGTGAGGTTTTCAACGCTAC |
| | Lmut13 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | F62W | GTTGAACGACCTGTGTTGGACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 39) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 100 | RBD- | ATCACCAACTTGTGTCCATTCGGTGAGGTTTTCAACGCTAC |
| | Lmut14 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | R78D | GTTGAACGACCTGTGTTTCACTAACGTCTACGCCGACTCCT |
| | (nucleotide | TCGTTATTAGAGGTGACGAGGTTGATCAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 40) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 101 | RBD- | ATCACCAACTTGTGTCCATTCGGTGAGGTTTTCAACGCTAC |
| | Lmut15 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | Q84A | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | GCTACTGGTAAGATCGCTGACTACAACTACAAGCTGCCAGA |
| | of SEQ ID | CGACTTCACCGGTTGTGTTATTGCTTGGAACTCCAACAACC |
| | NO: 41) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 102 | RBD- | ATCACCAACTTGTGTCCATTCGGTGAGGTTTTCAACGCTAC |
| | Lmut16 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | D98N | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CAATTTCACCGGTTGTGTTATTGCTTGGAACTCCAACAACC |
| | NO: 42) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 103 | RBD- | ATCACCAACTTGTGTCCATTCGGTGAGGTTTTCAACGCTAC |
| | Lmut17 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | L111I | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACA |
| | NO: 43) | TTGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 104 | RBD- | ATCACCAACTTGTGTCCATTCGGTGAGGTTTTCAACGCTAC |
| | Lmut18 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | Y178H | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 44) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCACATAG |
| | | AGTCGTCGTTTTGTCCTTCGAGTTGTTGCATGCTCCAGCTA |
| | | CTGTTTGCGGTCCAAAGAAGTCCACTAAC |
| 105 | RBD- | ATCACCAACTTGTGTCCATTCGGTGAGGTTTTCAACGCTAC |
| | Lmut19 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAGATGCTACGGTGTTTCCCCAACTAA |
| | K48R | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 45) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 106 | RBD- | ATCACCAACTTGTGTCCATTCGGTGAGGTTTTCAACGCTAC |
| | Lmut20 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | I72V | GTTGAACGACCTGTGTTTCACTAACGTCTACGCCGACTCCT |
| | (nucleotide | TCGTTGTTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 46) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 107 | RBD- | ATCACCAACTTGTGTCCATTCGGTGAGGTTTTCAACGCTAC |
| | Lmut21 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | Q168D | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 47) | TGGACTCCAAGGTTGGTGGTAACTACAATTACAAATACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCGATCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 108 | RBD- | ATCACCAACTTGTGTCCATTCGGTGAGGTTTTCAACGCTAC |
| | Lmut22 | TAGATTCGCTTCCGTTTACGCCTGGAACAGAAAGAGAATCT |
| | N1del | CCAACTGCGTTGCTGACTACTCCGTCTTGTACAACTCTGCT |
| | L122K | TCATTCTCCACCTTCAAGTGCTACGGTGTTTCCCCAACTAA |
| | L125N | GTTGAACGACCTGTGTTTCACTAACGTCTACGCCGACTCCT |
| | (nucleotide | TCGTTATTAGAGGTGACGAGGTTAGACAGATCGCTCCAGGT |
| | sequence | CAAACTGGTAAGATCGCTGACTACAACTACAAGCTGCCAGA |
| | of SEQ ID | CGACTTCACCGGTTGTGTTATTGCTTGGAACTCCAACAACC |
| | NO: 48) | TGGACTCCAAGGTTGGTGGTAACTACAATTACAAATACCGT |
| | | AATTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAAC |
| 109 | RBD-B | ATCACCAACTTGTGTCCATTCGGTGAGGTTTTCAACGCTAC |
| | N1del | TAGATTCGCTTCCGTTTACGCCTGGAACAGAAAGAGAATCT |
| | Histag | CCAACTGCGTTGCTGACTACTCCGTCTTGTACAACTCTGCT |
| | FreeCys | TCATTCTCCACCTTCAAGTGCTACGGTGTTTCCCCAACTAA |
| | (nucleotide | GTTGAACGACCTGTGTTTCACTAACGTCTACGCCGACTCCT |
| | sequence | TCGTTATTAGAGGTGACGAGGTTAGACAGATCGCTCCAGGT |
| | of SEQ ID | CAAACTGGTAAGATCGCTGACTACAACTACAAGCTGCCAGA |
| | NO: 49) | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACCATCATCATCAT |
| | | CATCATTGT |
| 110 | RBD-B | ATCACCAACTTGTGTCCATTCGGTGAGGTTTTCAACGCTAC |
| | N1del | TAGATTCGCTTCCGTTTACGCCTGGAACAGAAAGAGAATCT |
| | Long | CCAACTGCGTTGCTGACTACTCCGTCTTGTACAACTCTGCT |
| | Linker | TCATTCTCCACCTTCAAGTGCTACGGTGTTTCCCCAACTAA |
| | SpyTag | GTTGAACGACCTGTGTTTCACTAACGTCTACGCCGACTCCT |
| | | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAATCTACCAATGGTGGTGATGGT |
| | | GGCGACGGCGGAGATGGTGGTGCTCATATAGTTATGGTTGA |
| | | CGCCTACAAGCCTACTAAG |
| 111 | RBD-J | ATCACCAACTTGTGTCCATTCGGTGAGGTTTTCAACGCTAC |
| | N1del | TAGATTCGCTTCCGTTTACGCCTGGAACAGAAAGAGAATCT |
| | L122K | CCAACTGCGTTGCTGACTACTCCGTCTTGTACAACTCTGCT |
| | F160W | TCATTCTCCACCTTCAAGTGCTACGGTGTTTCCCCAACTAA |
| | Long | GTTGAACGACCTGTGTTTCACTAACGTCTACGCCGACTCCT |
| | Linker | TCGTTATTAGAGGTGACGAGGTTAGACAGATCGCTCCAGGT |
| | SpyTag | CAAACTGGTAAGATCGCTGACTACAACTACAAGCTGCCAGA |
| | (nucleotide | CGACTTCACCGGTTGTGTTATTGCTTGGAACTCCAACAACC |
| | sequence | TGGACTCCAAGGTTGGTGGTAATTACAATTACAAGTACAGG |
| | of SEQ ID | CTGTTCCGTAAGTCCAACCTGAAGCCATTCGAGAGAGACAT |
| | NO: 50) | CTCCACTGAGATCTACCAAGCTGGTTCCACTCCATGTAACG |
| | | GTGTTGAGGGTTTCAACTGTTACTGGCCATTGCAGTCTTAC |
| | | GGTTTCCAGCCAACTAACGGTGTCGGTTACCAACCATACAG |
| | | AGTTGTCGTTTTGTCCTTCGAGTTGTTGCACGCTCCAGCTA |
| | | CTGTTTGTGGTCCAAAGAAATCCACCAATGGTGGTGATGGT |
| | | GGCGACGGCGGAGATGGTGGTGCTCATATAGTTATGGTTGA |
| | | CGCCTACAAGCCTACTAAG |
| 112 | RBD-B | ATCACCAACTTGTGTCCATTCGGTGAGGTTTTCAACGCTAC |
| | 207C | TAGATTCGCTTCCGTTTACGCCTGGAACAGAAAGAGAATCT |
| | (nucleotide | CCAACTGCGTTGCTGACTACTCCGTCTTGTACAACTCTGCT |
| | sequence | TCATTCTCCACCTTCAAGTGCTACGGTGTTTCCCCAACTAA |
| | of SEQ ID | GTTGAACGACCTGTGTTTCACTAACGTCTACGCCGACTCCT |
| | NO: 51) | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACTTGGTCAAGAAC |
| | | AAATGT |
| 113 | RBD-J | ATCACCAACTTGTGTCCATTCGGTGAGGTTTTCAACGCTAC |
| | 207C | TAGATTCGCTTCCGTTTACGCCTGGAACAGAAAGAGAATCT |
| | (nucleotide | CCAACTGCGTTGCTGACTACTCCGTCTTGTACAACTCTGCT |
| | sequence | TCATTCTCCACCTTCAAGTGCTACGGTGTTTCCCCAACTAA |
| | of SEQ ID | GTTGAACGACCTGTGTTTCACTAACGTCTACGCCGACTCCT |
| | NO: 52) | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACAAATACCGT |
| | | TTGTTTAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTGGCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACTTGGTCAAGAAC |
| | | AAATGT |
| 114 | RBD-B | ATCACCAACTTGTGTCCATTCGGTGAGGTTTTCAACGCTAC |
| | C207S | TAGATTCGCTTCCGTTTACGCCTGGAACAGAAAGAGAATCT |
| | 259C | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 53) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACTTGGTCAAGAAC |
| | | AAATCTGTCAACTTTAACTTCAACGGCCTGACCGGTACTGG |
| | | TGTTTTGACTGAATCCAACAAGAAGTTCCTGCCATTCCAGC |
| | | AGTTCGGTAGAGACATTGCTGACACTACTGACGCCGTTAGA |
| | | GATCCACAGACTTTGGAGATCTTGGACATCACCCCATGT |
| 115 | RBD-B | ATCACCAACTTGTGTCCATTCGGTGAGGTTTTCAACGCTAC |
| | C207S | TAGATTCGCTTCCGTTTACGCCTGGAACAGAAAGAGAATCT |
| | 259CPPC | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 54) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACTTGGTCAAGAAC |
| | | AAATCTGTCAACTTTAACTTCAACGGCCTGACCGGTACTGG |
| | | TGTTTTGACTGAATCCAACAAGAAGTTCCTGCCATTCCAGC |
| | | AGTTCGGTAGAGACATTGCTGACACTACTGACGCCGTTAGA |
| | | GATCCACAGACTTTGGAGATCTTGGACATCACCCCATGTCC |
| | | ACCATGT |
| 116 | RBD-J | ATCACCAACTTGTGTCCATTCGGTGAGGTTTTCAACGCTAC |
| | C207S | TAGATTCGCTTCCGTTTACGCCTGGAACAGAAAGAGAATCT |
| | 259C | CCAACTGCGTTGCTGACTACTCCGTCTTGTACAACTCTGCT |
| | (nucleotide | TCATTCTCCACCTTCAAGTGCTACGGTGTTTCCCCAACTAA |
| | sequence | GTTGAACGACCTGTGTTTCACTAACGTCTACGCCGACTCCT |
| | of SEQ ID | TCGTTATTAGAGGTGACGAGGTTAGACAGATCGCTCCAGGT |
| | NO: 55) | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACAAATACCGT |
| | | TTGTTTAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTGGCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACTTGGTCAAGAAC |
| | | AAATCTGTCAACTTTAACTTCAACGGCCTGACCGGTACTGG |
| | | TGTTTTGACTGAATCCAACAAGAAGTTCCTGCCATTCCAGC |
| | | AGTTCGGTAGAGACATTGCTGACACTACTGACGCCGTTAGA |
| | | GATCCACAGACTTTGGAGATCTTGGACATCACCCCATGT |
| 117 | RBD-J | ATCACCAACTTGTGTCCATTCGGTGAGGTTTTCAACGCTAC |
| | C207S | TAGATTCGCTTCCGTTTACGCCTGGAACAGAAAGAGAATCT |
| | 259CPPC | CCAACTGCGTTGCTGACTACTCCGTCTTGTACAACTCTGCT |
| | | TCATTCTCCACCTTCAAGTGCTACGGTGTTTCCCCAACTAA |
| | | GTTGAACGACCTGTGTTTCACTAACGTCTACGCCGACTCCT |
| | | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACAAATACCGT |
| | | TTGTTTAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTGGCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACTTGGTCAAGAAC |
| | | AAATCTGTCAACTTTAACTTCAACGGCCTGACCGGTACTGG |
| | | TGTTTTGACTGAATCCAACAAGAAGTTCCTGCCATTCCAGC |
| | | AGTTCGGTAGAGACATTGCTGACACTACTGACGCCGTTAGA |
| | | GATCCACAGACTTTGGAGATCTTGGACATCACCCCATGTCC |
| | | ACCATGT |
| 118 | RBD-B 260 | ATCACCAACTTGTGTCCATTCGGTGAGGTTTTCAACGCTAC |
| | B-Long | TAGATTCGCTTCCGTTTACGCCTGGAACAGAAAGAGAATCT |
| | (nucleotide | CCAACTGCGTTGCTGACTACTCCGTCTTGTACAACTCTGCT |
| | sequence | TCATTCTCCACCTTCAAGTGCTACGGTGTTTCCCCAACTAA |
| | of SEQ ID | GTTGAACGACCTGTGTTTCACTAACGTCTACGCCGACTCCT |
| | NO: 56) | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACCTGGTCAAGAAC |
| | | AAGTGCGTCAACTTTAACTTCAACGGTCTGACCGGTACTGG |
| | | TGTTTTGACTGAGTCCAACAAGAAGTTCCTGCCATTCCAGC |
| | | AATTCGGTAGAGACATTGCTGACACTACTGACGCCGTTAGA |
| | | GATCCACAGACTTTGGAGATCTTGGACATCACCCCATGTTC |
| | | T |
| 119 | RBD-J 260 | ATCACCAACTTGTGTCCATTCGGTGAGGTTTTCAACGCTAC |
| | J-Long | TAGATTCGCTTCCGTTTACGCCTGGAACAGAAAGAGAATCT |
| | (nucleotide | CCAACTGCGTTGCTGACTACTCCGTCTTGTACAACTCTGCT |
| | sequence | TCATTCTCCACCTTCAAGTGCTACGGTGTTTCCCCAACTAA |
| | of SEQ ID | GTTGAACGACCTGTGTTTCACTAACGTCTACGCCGACTCCT |
| | NO: 57) | TCGTTATTAGAGGTGACGAGGTTAGACAGATCGCTCCAGGT |
| | | CAAACTGGTAAGATCGCTGACTACAACTACAAGCTGCCAGA |
| | | CGACTTCACCGGTTGTGTTATTGCTTGGAACTCCAACAACC |
| | | TGGACTCCAAGGTTGGTGGTAACTACAATTACCTGTACCGT |
| | | CTGTTCAGAAAGTCCAACCTGAAGCCATTCGAGAGAGACAT |
| | | CTCCACTGAGATCTACCAAGCTGGTTCCACTCCATGTAACG |
| | | GTGTTGAGGGTTTCAACTGTTACTTCCCATTGCAGTCCTAC |
| | | GGTTTCCAGCCAACTAATGGTGTTGGTTACCAGCCATACAG |
| | | AGTCGTCGTTTTGTCCTTCGAGTTGTTGCATGCTCCAGCTA |
| | | CTGTTTGCGGTCCAAAGAAGTCCACTAACCTGGTCAAGAAC |
| | | AAGTGCGTCAACTTTAACTTCAACGGTCTGACCGGTACTGG |
| | | TGTTTTGACTGAGTCCAACAAGAAGTTCCTGCCATTCCAGC |
| | | AATTCGGTAGAGACATTGCTGACACTACTGACGCCGTTAGA |
| | | GATCCACAGACTTTGGAGATCTTGGACATCACCCCATGTTC |
| | | T |

Other embodiments of the S glycoprotein variant are described in US Published patent Application US 2022-0133880, incorporated by reference herein in its entirety.

### Optional Non-Liposome, Non-Micelle Particle

In one embodiment, the composition further comprises a non-liposome, non-micelle particle, wherein the particle comprises a lipid, a sterol, a saponin, and an optional additional non-alum adjuvant, wherein the particle is optionally bound to the alum.

The particle is optionally bound to the alum. In one embodiment, the alum and the particle are not bound. In another embodiment, the alum and particle are bound. When bound, the alum and particle may be covalently or non-covalently bound. In one embodiment, the particle is covalently bound to the alum via phosphate residues in the particle.

The particle is a non-liposome, non-micelle particle, wherein the particle comprises a lipid, a sterol, a saponin, and an optional additional non-alum adjuvant. Such particles are described, for example, in published US patent application 20200085756, incorporated by reference herein in its entirety.

In one embodiment the particle is a porous, cage-like nanoparticle comprising saponin, sterol, lipid, and an optional additional adjuvant. Exemplary saponins, sterols, lipids, additional adjuvants including TLR4 agonists, and antigens are discussed in more detail below.

Generally, the nanocage particle is formed by mixing the components together in the presence of a detergent in a suitable ratio such that when the detergent is removed (e.g., by dialysis), the components self-assemble into nanocages. The size of the nanocages is typically dictated by the properties of the components and the self-assembly process. The disclosed compositions and methods typically yield nanocages in the range of about 30 nm and about 60 nm, or about 40 nm to about 50 nm, with an exemplary size being about 40 nm. The nanocages generally assume a distinctive porous morphology that can be structurally distinguished by transmission electronic microscope (TEM) from lipid monolayer (micelle) and lipid bilayer (liposome) particles. The particles are not micelles or liposomes.

### A. Saponin

The particles include one or more saponins. A suitable saponin is one that can induce or enhance an immune response. Saponins from plants have proven to be very effective as adjuvants. Saponins are triterpene and steroid glycosides widely distributed in the plant kingdom. Structurally, saponins are amphiphilic surfactants, which explains their surfactant properties, ability to form colloidal solutions, hemolytic activity and ability to form mixed micelles with lipids and sterols. The saponins most studied and used as adjuvants are those from Chilean tree Quillaja saponaria, which have cellular and humoral adjuvant activity. Saponins extracts from Quillaja saponaria with adjuvant activity are known and employed in commercial or experimental vaccines formulation.

A particular saponin preparation is called Quil A®, a saponin preparation isolated from the South American tree Quillaja Saponaria Molina and was first described by Dalsgaard et al. in 1974 (“Saponin adjuvants,” Archiv. für die gesamte Virus forschung, Vol. 44, Springer Verlag, Berlin, p 243-254) to have adjuvant activity. The isolation of pure saponins or better defined mixtures from the Quil A® product having adjuvant activity and lower toxicity than Quil A® have also been described. Purified fragments of Quil A® that retain adjuvant activity without the toxicity associated with Quil A® (EP 0362 278), for example QS7 and QS21 (also known as QA7 and QA21), have been isolated by HPLC. QS-21 is a natural saponin derived from the bark of Quillaja Saponaria Molina, which induces CD8+ cytotoxic T cells (CTLs), Th1 cells and a predominant IgG2a antibody response. QS-21 has been used or is being studied as an adjuvant for various types of vaccines. See also EP 0 362 279 B1 and U.S. Pat. No. 5,057,540.

The isolation and adjuvant activity of other isolated Quil A® saponins, including those called QS-17, and 18 have also been reported, and can also be used in the disclosed nanocages

In other embodiments, the saponin is from Quillaja brasiliensis (A. St.-Hil. et Tul.) Mart., which is native to southern Brazil and Uruguay and has saponins that have proven to be effective as adjuvants with a similar activity against viral antigens as Quil A® (Silveira et al., Vaccine 29 (2011), 9177-9182).

Other useful saponins are derived from the plants Aesculus hippocastanum or Gyophila Struthium. Other saponins which have been described in the literature include escin, which has been described in the Merck index (12th ed: entry 3737) as a mixture of saponins occurring in the seed of the horse chestnut tree, Lat: Aesculus hippocastanum. Its isolation by chromatography and purification (Fiedler, Arzneimittel-Forsch. 4, 213 (1953)), and by ion exchange resins (Erbring et al., U.S. Pat. No. 3,238,190) has been described. Fractions of escin have been purified and shown to be biologically active (Yoshikawa M, et al. (Chem Pharm Bull (Tokyo) August 1996; 44(8): 1454-1464)). Sapoalbin from Gypsophila struthium (R. Vochten et al., 1968, J. Pharm. Belg., 42, 213-226) has also been described.

In other embodiments, the saponin is a synthetic saponin. See, e.g., U.S. Published Application No. 2011/0300177 and U.S. Pat. No. 8,283,456, which describe the Triterpene Saponin Synthesis Technology (TriSST) platform, a convergent synthetic approach in which the four domains in QS-21 (branched trisaccharide+triterpene+linear tetrasaccharide+fatty acyl chain) are synthesized separately and then assembled to produce the target molecule. Each of the domains can be modified independently and then combined to produce a virtually infinite number of rationally designed QS-21 analogs. Initially, fully synthetic QS-21(SQS-21) was shown to be safe and immunologically active in a Phase 1 clinical trial, and later over 100 analogues were prepared and tested in a systematic sequential series of studies. See, e.g., Ragupathi, et al., Expert Rev Vaccines. 2011 April; 10(4): 463-470. See also Zu, et al., Journal of Carbohydrate Chemistry, Volume 33, 2014-Issue 6, pages 269-97.

Preferably the saponin component is in a substantially pure form, for example, at least 90% pure, preferably at least 95% pure and most preferably at least 98% pure.

### B. Sterol

The particles include one or more sterols. Sterols include p-sitosterol, stigmasterol, ergosterol, ergocalciferol, campesterol, and cholesterol. These sterols are well known in the art, for example cholesterol is disclosed in the Merck Index, 11th Ed., page 341, as a naturally occurring sterol found in animal fat. In preferred embodiments, the sterol is cholesterol or a derivative thereof e.g., ergosterol or cholesterylhemisuccinate.

### C. Lipid

The particles include one or more lipids, such as one or more phospholipids. The lipid can be neutral, anionic, or cationic at physiologic pH. Phospholipids include, but are not limited to, diacylglycerides such as phosphatidic acid (phosphatidate) (PA), phosphatidylethanolamine (cephalin) (PE), phosphatidylcholine (lecithin) (PC), phosphatidylserine (PS), and phosphoinositides, e.g., phosphatidylinositol (PI), phosphatidylinositol phosphate (PIP), phosphatidylinositol bisphosphate (PIP2) and phosphatidylinositol trisphosphate (PIP3), as well as phosphoshingolipids such as ceramide phosphorylcholine (Sphingomyelin) (SPH), ceramide phosphorylethanolamine (Sphingomyelin) (Cer-PE), and ceramide phosphoryllipid, and natural and synthetic phospholipid derivatives such as egg PC (Egg lecithin), egg PG, soy PC, hydrogenated soy PC, sphingomyelin, phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), and PEG phospholipid (mPEG-phospholipid, polyglycerin-phospholipid, functionalized-phospholipid, terminal activated-phospholipid).

Thus, particles can include any one of more of 1,2-Didecanoyl-sn-glycero-3-phosphocholine (DDPC), 1,2-Dierucoyl-sn-glycero-3-phosphate (Sodium Salt) (DEPA-NA), 1,2-Dierucoyl-sn-glycero-3-phosphocholine (DEPC), 1,2-Dierucoyl-sn-glycero-3-phosphoethanolamine (DEPE) 1,2-Dierucoyl-sn-glycero-3[Phospho-rac-(1-glycerol) (Sodium Salt) (DEPG-NA), 1,2-Dilinoleoyl-sn-glycero-3-phosphocholine (DLOPC), 1,2-Dilauroyl-sn-glycero-3-phosphate (Sodium Salt) (DLPA-NA) 1,2-Dilauroyl-sn-glycero-3-phosphocholine (DLPC) 1,2-Dilauroyl-sn-glycero-3-phosphoethanolamine (DLPE), 1,2-Dilauroyl-sn-glycero-3[Phospho-rac-(1-glycerol) (Sodium Salt) (DLPG-NA), 1,2-Dilauroyl-sn-glycero-3[Phospho-rac-(1-glycerol) (Ammonium Salt) (DLPG-NH4), 1,2-Dilauroyl-sn-glycero-3-phosphoserine (Sodium Salt) (DLPS-NA), 1,2-Dimyristoyl-sn-glycero-3-phosphate (Sodium Salt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol) (Sodium Salt) (DMPG-NA), 1,2-Dimyristoyl-sn-glycero-3[Phospho-rac-(1-glycerol) (Ammonium Salt) (DMPG-NH4), 1,2-Dimyristoyl-sn-glycero-3 [Phospho-rac-(1-glycerol) (Sodium/Ammonium Salt) (DMPG-NH4/NA), 1,2-Dimyristoyl-sn-glycero-3-phosphoserine (Sodium Salt) (DMPS-NA), 1,2-Dioleoyl-sn-glycero-3-phosphate (Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol) (Sodium Salt) (DOPG-NA), 1,2-Dioleoyl-sn-glycero-3-phosphoserine (Sodium Salt) (DOPS-NA), 1,2-Dipalmitoyl-sn-glycero-3-phosphate (Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol) (Sodium Salt) (DPPG-NA), 1,2-Dipalmitoyl-sn-glycero-3 [Phospho-rac-(1-glycerol) (Ammonium Salt) (DPPG-NH4), 1,2-Dipalmitoyl-sn-glycero-3-phosphoserine (Sodium Salt) (DPPS-NA), 1,2-Distearoyl-sn-glycero-3-phosphate (Sodium Salt) (DSPA-NA), 1,2-Distearoyl-sn-glycero-3-phosphocholine (DSPC), 1,2-Distearoyl-sn-glycero-3-phosphoethanolamine (DSPE), 1,2-Distearoyl-sn-glycero-3[Phospho-rac-(1-glycerol) (Sodium Salt) (DSPG-NA), 1,2-Distearoyl-sn-glycero-3[Phospho-rac-(1-glycerol) (Ammonium Salt) (DSPG-NH4), 1,2-Distearoyl-sn-glycero-3-phosphoserine (Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LYS OPC MYRISTIC), 1-Palmitoyl-sn-glycero-3-phosphocholine (LYS OPC PALMITIC), 1-Stearoyl-sn-glycero-3-phosphocholine (LYS OPC STEARIC), 1-Myristoyl-2-palmitoyl-sn-glycero 3-phosphocholine (Milk Sphingomyelin MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol) . . . ] (Sodium Salt) (POPG-NA), 1-Palmitoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). Any of the lipids can be PEGylated lipids, for example PEG-DSPE. In a specific embodiment, the phospholipid is 2-Dipalmitoyl-snglycero-3-phosphocholine (DPPC).

### D. Adjuvant

The particles may optionally include one or more additional adjuvants. In one embodiment, the particle comprises an additional adjuvant. The additional adjuvant typically has physical and biochemical properties compatible with its incorporation into structure of the particle and that do not prevent particle self-assembly. The additional adjuvant also typically increases at least one immune response relative to the same nanocage formulation in the absence of the additional adjuvant. Immune responses include, but are not limited to, an increase in an antigen-specific antibody response (e.g., IgG, IgG2a, IgG1, or a combination thereof), an increase in a response in germinal centers (e.g., increase in the frequency of germinal center B cells, an increase in frequencies and/or activation of T follicular helper (Tfh) cells, an increase in B cell presence or residence in dark zone of germinal center or a combination thereof), an increase in plasmablast frequency, an increase in inflammatory cytokine expression (e.g., IL-6, IFN-γ, IFN-α, IL-1β, TNF-α, CXCL10 (IP-10), or a combination thereof), an increase in drainage of antigen from the injection site, an in increase in antigen accumulation in the lymph nodes, an increase in lymph node permeability, an increase in lymph flow, an increase in antigen-specific B cell antigen uptake in lymph nodes, an increase in humoral responses beyond the proximal lymph node, increased diffusion of antigen into B cell follicles, or a combination thereof, when the nanocages are administered to a subject, preferably in combination with an antigen.

1. TLR4 Agonists

In some embodiments, the additional adjuvant is a TLR agonist. TLR4 is a transmembrane protein member of the toll-like receptor family, which belongs to the pattern recognition receptor (PRR) family. Its activation leads to an intracellular signaling pathway NF-κB and inflammatory cytokine production responsible for activating the innate immune system. Classes of TLR agonists include, but are not limited to, viral proteins, polysaccharides, and a variety of endogenous proteins such as low-density lipoprotein, beta-defensins, and heat shock protein.

Exemplary TLR4 agonist include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland).

In another embodiment, the TLR4 agonist is a natural or synthetic lipopolysaccharide (LPS), or a lipid A derivative thereof such as MPLA or 3D-MPLA. Lipopolysaccharides are the major surface molecule of, and occur exclusively in, the external leaflet of the outer membrane of gram-negative bacteria. LPS impede destruction of bacteria by serum complements and phagocytic cells, and are involved in adherence for colonization. LPS are a group of structurally related complex molecules of approximately 10,000 Daltons in size and contain three covalently linked regions: (i) an O-specific polysaccharide chain (O-antigen) at the outer region (ii) a core oligosaccharide central region (iii) lipid A—the innermost region which serves as the hydrophobic anchor, it includes glucosamine disaccharide units which carry long chain fatty acids.

The biological activities of LPS, such as lethal toxicity, pyrogenicity and adjuvanticity, have been shown to be related to the lipid A moiety. In contrast, immunogenicity is associated with the 0-specific polysaccharide component (O-antigen). Both LPS and lipid A have long been known for their strong adjuvant effects, but the high toxicity of these molecules has precluded their use in vaccine formulations. Significant effort has therefore been made towards reducing the toxicity of LPS or lipid A while maintaining their adjuvanticity.

The Salmonella minnesota mutant R595 was isolated in 1966 from a culture of the parent (smooth) strain (Luderitz et al. 1966 Ann. N. Y. Acad. Sci. 133:349-374). The colonies selected were screened for their susceptibility to lysis by a panel of phages, and only those colonies that displayed a narrow range of sensitivity (susceptible to one or two phages only) were selected for further study. This effort led to the isolation of a deep rough mutant strain which is defective in LPS biosynthesis and referred to as S. minnesota R595.

In comparison to other LPS, those produced by the mutant S. minnesota R595 have a relatively simple structure. (i) they contain no O-specific region—a characteristic which is responsible for the shift from the wild type smooth phenotype to the mutant rough phenotype and results in a loss of virulence (ii) the core region is very short—this characteristic increases the strain susceptibility to a variety of chemicals (iii) the lipid A moiety is highly acylated with up to 7 fatty acids.

4′-monophosporyl lipid A (MPLA), which may be obtained by the acid hydrolysis of LPS extracted from a deep rough mutant strain of gram-negative bacteria, retains the adjuvant properties of LPS while demonstrating a toxicity which is reduced by a factor of more than 1000 (as measured by lethal dose in chick embryo eggs) (Johnson et al. 1987 Rev. Infect. Dis. 9 Suppl:S512-S516). LPS is typically refluxed in mineral acid solutions of moderate strength (e.g. 0.1 M HCl) for a period of approximately 30 minutes. This process results in dephosphorylation at the 1 position, and decarbohydration at the 6′ position, yielding MPLA. In some embodiments, the TLR4 agonist is MPLA.

3-O-deacylated monophosphoryl lipid A (3D-MPLA), which can be obtained by mild alkaline hydrolysis of MPLA, has a further reduced toxicity while again maintaining adjuvanticity, see U.S. Pat. No. 4,912,094 (Ribi Immunochemicals). Alkaline hydrolysis is typically performed in organic solvent, such as a mixture of chloroform/methanol, by saturation with an aqueous solution of weak base, such as 0.5 M sodium carbonate at pH 10.5. In some embodiments, the TLR4 agonist is 3D-MPLA.

In some embodiments, the MPLA is a fully synthetic MPLA such as Phosphorylated HexaAcyl Disaccharide (PHAD®), the first fully synthetic monophosphoryl Lipid A available for use as an adjuvant in human vaccines, or Monophosphoryl 3-Deacyl Lipid A (Synthetic) (3D-PHAD®). See also U.S. Pat. No. 9,241,988.

2. Other Exemplary Adjuvants

As introduced above, the additional adjuvant typically has physical and biochemical properties compatible with its incorporation into the structure of the particle and that do not prevented particle self-assembly and increase an immune response. Thus, other suitable adjuvants immunostimulators include those that include a lipid tail, or can be modified to contain a lipid tail. Examples of molecules that include a lipid tail, or can be modified to include one, can be, for example, pathogen-associated molecular patterns (PAMPs). PAMPS are recognized by pattern recognition receptors (PRRs). Five families of PRRs have been shown to initiate pro-inflammatory signaling pathways: Toll-like receptors (TLRs), NOD-like receptors (NLRs), RIG-I-like receptors (RLRs), C-type lectin receptors (CLRs) and cytosolic dsDNA sensors (CDSs). Also, some NLRs are involved in the formation of pro-inflammatory complexes called inflammasomes.

Thus, in some embodiments, the additional adjuvant is a TLR ligand, a NOD ligand, an RLR ligand, a CLR ligand, and inflammasome inducer, a STING ligand, or a combination thereof. Such ligands are known in the art can obtained through commercial vendors such as InvivoGen.

As introduced above, the ligands and other adjuvants can be modified (e.g., through chemical conjugation, for example, maleimide thiol reaction, amine N-hydroxysuccinimide ester reaction, click chemistry, etc.) to include a lipid tail to facilitate incorporation of the adjuvant into the nanocage structure during self-assembly. Preferred lipids will include a 16:0 dipalmitoyl tail such as 1,2-dipalmitoyl-sn-glycero-3-phosphoethanolamine-N-[4-(p-maleimidophenyl)butyramide], these, however, are non-limiting examples. For example, lipids of different lengths are also contemplated. In preferred embodiments, the lipid or lipids is/are unsaturated. Chemically functionalized lipids that that can be used for conjugation are known in the art and commercially available. See, for example, AVANTI® Polar Lipids, Inc. (e.g., “Headgroup Modified Lipids” and “Functionalized Lipids”).

The additional adjuvant can be an immunostimulatory oligonucleotide, preferable a lipidated immunostimulatory oligonucleotide. Exemplary lapidated immunostimulatory oligonucleotides and methods of making them are described in Liu, et al., Nature Letters, 507:519-22 (+11 pages of extended data) (2014)) (lipo-CpG) and U.S. Pat. No. 9,107,904, that contents of which are incorporated by reference herein in their entireties. In some embodiments, the immunostimulatory oligonucleotide portion of the adjuvant can serve as a ligand for PRRs. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76(5):2123-2129, (2008)). Therefore, the sequence of the oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDAS), which are best known as RNA-sensing receptors in the cytosol. Therefore, in some embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In some embodiments, the oligonucleotide includes two or more immunostimulatory sequences.

Microbial cell-wall components such as Pam2CSK4, Pam3CSK4, and flagellin activate TLR2 and TLR5 receptors respectively and can also be used.

Any suitable ratios of the various particle components may be used. In one embodiment, comprising a lipid:additional adjuvant:sterol:saponin molar ratio of 2.5:1:10:10, or a variation thereof wherein the molar ratio of lipid, additional adjuvant, sterol, saponin or any combination thereof is increased or decreased by any value between about 0 and about 3. In a specific embodiment, the lipid is DPPC, the additional adjuvant is a natural or synthetic MPLA, the sterol is cholesterol, and the saponin is Quil A® in a molar ratio of 2.5:1:10:10. In another embodiment the Quil-A:chol:DPPC:MPLA are in a mass ratio of 10:2:1:1. See US20200085756 for exemplary methods for modifying the molar ratio or mass ratio of the particle components.

### Pharmaceutical Compositions

In a further embodiment, the disclosure provides pharmaceutical compositions comprising the composition of any embodiment of the disclosure, and a pharmaceutically acceptable carrier. In this embodiment, the compositions are combined with a pharmaceutically acceptable carrier. Suitable acids which are capable of forming such salts include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming such salts include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

In some embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In some embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In some embodiments, the optimal pharmaceutical composition will be determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the immunogenic composition.

In some embodiments, the primary vehicle or carrier in a pharmaceutical composition can be either aqueous or non-aqueous in nature. For example, in some embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In some embodiments, pharmaceutical compositions comprise Tris buffer of about pH 7.0-8.5, or acetate buffer of about pH 4.0-5.5, which can further include sorbitol or a suitable substitute therefore. In some embodiments, an immunogenic composition can be prepared for storage by mixing the selected composition having the desired degree of purity with optional formulation agents (Remington's Pharmaceutical Sciences, supra) in the form of a lyophilized cake or an aqueous solution. Further, in some embodiments, an immunogenic composition can be formulated as a lyophilizate using appropriate excipients such as sucrose.

The pharmaceutical compositions of the invention may be made up in any suitable formulation, preferably in formulations suitable for administration by parenteral delivery such as subcutaneous of intra-venous injection, inhalation, or oral delivery. Such pharmaceutical compositions can be used, for example, in the therapeutic methods disclosed herein.

The pharmaceutical compositions may contain any other components as deemed appropriate for a given use. In another embodiment, the disclosure provides vaccines comprising the composition of any embodiment of the disclosure in which an antigen is present. The compositions and vaccines may be used, for example in the methods of the disclosure.

In another aspect, the disclosure provides methods for generating an immune response against a SARS-CoV-2 S glycoprotein variant, comprising administering to a subject an amount effective to generate an immune response in the subject of the composition of any embodiment herein.

The “immune response” refers to responses that induce, increase, or perpetuate the activation or efficiency of innate or adaptive immunity. The immune response includes, but is not limited to, the production of antibodies and/or cytokines and/or the activation of cytotoxic T cells, antigen presenting cells, helper T cells, dendritic cells and/or other cellular responses.

In a further embodiment, the disclosure provides methods of treating a subject in need thereof comprising administering to the subject the composition or vaccine of any embodiment herein in an effective amount to induce an immune response against the SARS-CoV-2 S glycoprotein variant.

In some embodiments, the immunogenic compositions are administered as part of prophylactic vaccines or immunogenic compositions which confer resistance in a subject to subsequent exposure to SARS-CoV-2, or as part of therapeutic vaccines, which can be used to initiate or enhance a subject's immune response to SARS-CoV-2 exposure. The desired outcome of a prophylactic or therapeutic immune response may vary according to the subject to be treated. For example, an immune response against SARS-CoV-2 may completely prevent colonization and replication of the virus, affecting “sterile immunity” and the absence of any disease symptoms. However, a vaccine against SARS-CoV-2 may be considered effective if it reduces the number, severity or duration of symptoms; if it reduces the number of individuals in a population with symptoms; or reduces the transmission of SARS-CoV-2.

Methods for analyzing an antibody response in a subject are known to those of skill in the art. For example, in some embodiments an increase in an immune response is measured by ELISA assays to determine antigen-specific antibody titers. In some embodiments, the methods increasing broadly neutralizing antibodies in a subject. Methods for measuring neutralizing antibodies are known to those of ordinary skill in the art. In some embodiments, elicitation of neutralizing antibodies is measured in a neutralization assay. Methods for identifying and measuring neutralizing antibodies are known to those of skill in the art. Neutralizing antibodies are an indicator of the protective efficacy of a vaccine, but direct protection from a sub-lethal or lethal challenge of virus unequivocally demonstrates the efficacy of the vaccine. In an exemplary animal model system, a virus challenge is conducted wherein the subjects are immunized, optionally more than once, and challenged after immune response to the vaccine has developed. Elicitation of neutralization may be quantified by measurement of morbidity or mortality on the challenged subjects.

In some embodiments, the administration of the composition or vaccine induces an improved B-memory cell response in immunized subjects. An improved B-memory cell response is intended to mean an increased frequency of peripheral blood B lymphocytes capable of differentiation into antibody-secreting plasma cells upon antigen encounter as measured by stimulation of in vitro differentiation. In some embodiments, the methods increase the number of antibody secreting B cells. In some embodiments, the antibody secreting B cells are bone marrow plasma cells, or germinal center B cells. In some embodiments, methods for measuring the number of antibody secreting B cells, includes, but are not limited to, an antigen-specific ELISPOT assay and flow cytometric studies of plasma cells, or germinal center B cells collected at various time points post-immunization.

In some embodiments, the disclosure provides methods of reducing a SARS-CoV-2 infection in a subject in need thereof, comprising administering to the subject an immunogenic composition or vaccine described herein. In some embodiments, the disclosure provides methods for inducing an anti-SARS-CoV-2 response in a subject with cancer, comprising administering to the subject an immunogenic composition or vaccine described herein.

The “subject” may be any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

In all embodiments, administration of the compositions may be by any suitable route, including but not limited to subcutaneous, intramuscular, intradermal, or intravenous injection.

### EXAMPLES

### Summary:

There remains a significant need for strategies to facilitate global SARS-CoV-2 vaccine coverage. To this end, subunit vaccines are attractive for their ability to be produced at low cost, at scale and without the need for ultra-cold storage temperatures, but the global supply of adjuvants for accessible vaccines is unclear. The most common clinical vaccine adjuvant, alum, is well-suited to global vaccination campaigns due to its manufacturability and low cost, but alum has exhibited relatively poor immunogenicity with SARS-CoV-2 subunit vaccines to date (Yang et al. Nature 2020, Dai et al. Cell 2020). Of equal importance to these practical issues is the ability of vaccines to promote neutralizing responses to SARS-CoV-2 variants that are now circulating globally. A number of preclinical studies have demonstrated that vaccines eliciting higher levels of neutralizing responses against the original Wuhan-Hu-1 virus tend to also elicit high neutralizing titers against these viral variants (Planas et al. Nature Medicine 2021). Hence, approaches to enhance the immunogenicity of alum-adjuvanted subunit vaccines for SARS-CoV-2 may be important in the effort to achieve global vaccination coverage.

We recently described an approach to augment alum:protein subunit vaccines by site-specific introduction of phosphoserine (pSer) peptide tags onto human immunodeficiency virus (HIV) protein immunogens (Moyer et al. Nature Medicine 2020). pSer tagging allows immunogens to bind to the surface of aluminum hydroxide via a ligand exchange reaction, providing tight binding that can be tuned by the valency of the pSer peptide tag sequence. Stable anchoring to alum was shown to prolong antigen delivery to lymph nodes via slow trafficking of alum particles, coincident with direct B cell triggering by antigen multivalently displayed on alum. These changes in the physical chemistry of vaccine delivery enhanced germinal center (GC) responses, serum antibody titers, and neutralizing antibody titers against HIV envelope (Env) immunogens.

Despite these promising data, alum remains an adjuvant that does not stimulate many of the innate immune recognition pathways that might be exploited to drive robust immune responses. We hypothesized that phosphate-mediated binding could be used to co-anchor SARS-CoV-2 and other antigens alongside complementary molecular adjuvants to alum particles to synergistically drive humoral immunity. To test this idea, we evaluated the potential of pSer-tagging to enhance the immunogenicity of alum:RBD subunit vaccines. We assessed alum binding, antigen structural stability, and in vivo humoral immune responses for pSer-modified RBD proteins. Immunization with pSer-labeled RBD antigens was found to greatly enhance the immunogenicity of this antigen in combination with alum. To further amplify these responses, we combined pSer-tagged RBD with the saponin/phospholipid nanoparticle adjuvant (SMNP) which intrinsically contains phosphate residues, for co-adsorption to alum. We found that the persistence of the SMNP adjuvant in vivo could be significantly increased by complexing with pSer-RBD and alum, correlating with synergistic enhancements in vaccine immunogenicity. These findings indicate that phosphoserine modification is a promising way to enhance the efficacy of SARS-CoV-2 subunit vaccines, and that combining alum with molecular adjuvants capable of undergoing ligand-exchange-mediated binding such as SMNP can further substantially potentiate humoral immunity.

While alum alone is a weak adjuvant, the combination of SMNP and alum synergistically enhanced humoral immune responses. Given the wide availability of alum as an adjuvant, combining SMNP with alum is feasible and direct means to enhance immune responses to immunization. Phosphate-mediated co-anchoring of antigen and SMNP to alum is an effective strategy to enhance the efficacy of SARS-CoV-2 vaccines and subunit vaccines more broadly. This may enable the reduction in total vaccine dose required to elicit protective responses.

### Summary

There is a need for additional rapidly scalable, low-cost vaccines against SARS-CoV-2 to achieve global vaccination. Aluminum hydroxide (alum) adjuvant is the most widely available vaccine adjuvant but elicits modest humoral responses. We hypothesized that phosphate-mediated co-anchoring of the receptor binding domain (RBD) of SARS-CoV-2 immunogen together with molecular adjuvants on alum particles could potentiate humoral immunity by promoting extended vaccine kinetics and co-delivery of vaccine components to lymph nodes. Modification of RBD immunogens with phosphoserine (pSer) peptides enabled efficient alum binding and slowed antigen clearance in vivo, leading to striking increases in germinal center responses and neutralizing antibody titers in mice. Adding phosphate-containing CpG or saponin adjuvants to pSer-RBD:alum immunizations synergistically enhanced vaccine immunogenicity, inducing neutralizing responses against SARS-CoV-2. Thus, phosphate-mediated co-anchoring of RBD and molecular adjuvants to alum is an effective strategy to enhance the efficacy of SARS-CoV-2 subunit vaccines.

### Results

### Phosphoserine Peptide Modification Facilitates Stable Binding of SARS-CoV-2 RBD to Alum

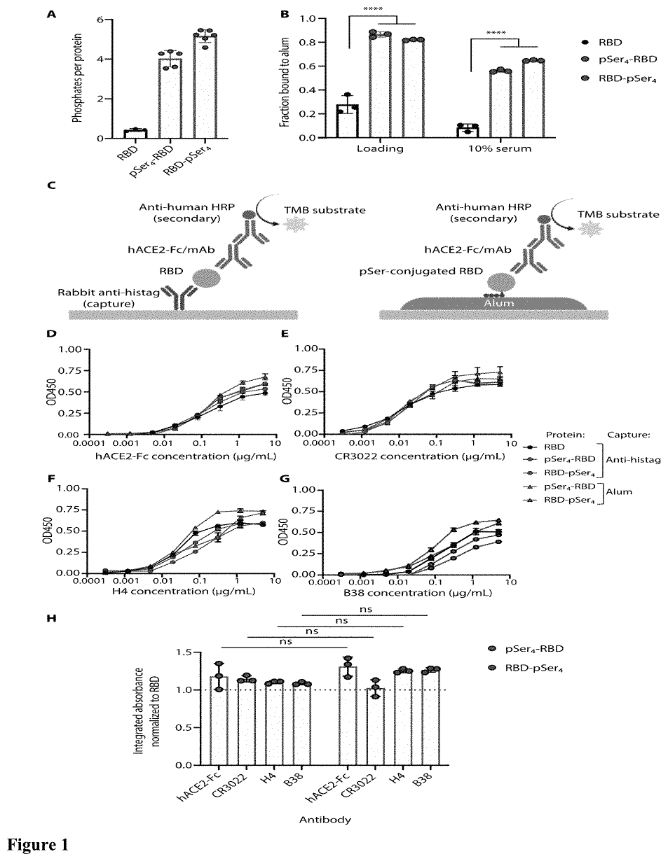

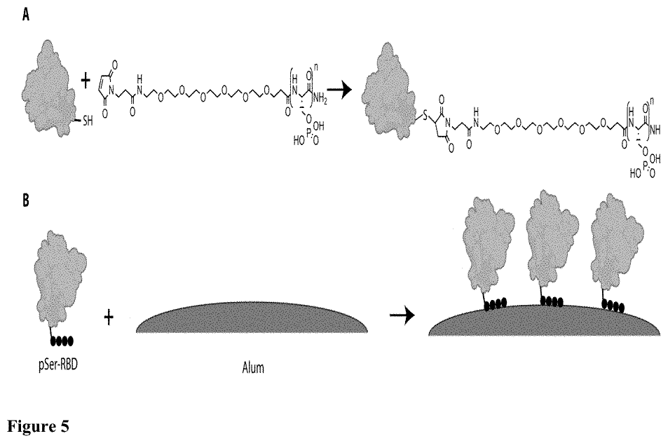

We first tested whether coupling a pSer peptide tag to Wuhan-Hu-1 SARS-CoV-2 RBD could engender stable binding to alum without disrupting key epitopes on the antigen. RBD (amino acids 332-532 of SARS-CoV-2 S protein, table 4) modified with a histag for purification and containing an N- or C-terminal free cysteine was expressed in yeast, and then conjugated with a peptide tag containing a maleimide group linked to a 6-unit poly(ethylene glycol) spacer followed by four phosphoserine residues (FIG. 5). Measurement of the mean number of phosphates per protein using a malachite green assay revealed that the N-terminal-coupled pSer₄-RBD (SEQ ID NO: 120-RBD) and C-terminal-modified RBD-pSer₄ (RBD-SEQ ID NO: 120) gained the expected ˜4 phosphates per protein (FIG. 1A). Incubation of unmodified RBD with alum in tris-buffered saline led to adsorption of only ˜25% of the antigen (FIG. 1B “loading”), most of which desorbed following a 24-hour incubation of the alum in phosphate buffer containing 10% serum (FIG. 1B “10% serum”). By contrast, both pSer-RBDs exhibited high levels of alum binding in buffer, with the majority remaining bound after serum/phosphate exposure (FIG. 1B). To confirm that the alum-bound RBD conjugates were structurally intact relative to unmodified protein, we used a modified sandwich ELISA approach to probe the antigenicity of the constructs. Unmodified RBD was captured on plates coated with a histag-specific antibody, while pSer-conjugated RBD was captured on alum-coated plates (FIG. 1C). The immobilized RBD was then probed for binding to serial dilutions of recombinant hACE2 protein, the target receptor recognized by RBD, or monoclonal antibodies CR3022 (which recognizes a highly conserved epitope distal from the receptor binding site (28)), H4, or B38. As shown in FIGS. 1D-H, the pSer-modified RBDs had antigenicity profiles indistinguishable from unmodified RBD, and the proteins captured on alum retained recognition of both probes. Thus, pSer modification allowed substantially enhanced RBD binding to alum without disrupting its structure.

N-Terminal pSer Modification Enhances the Immunogenicity of RBD Antigens

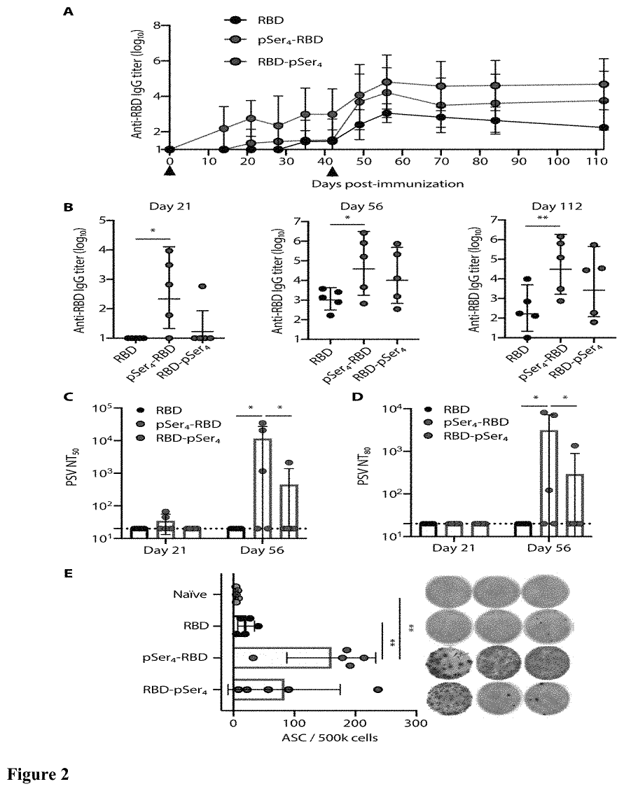

We immunized BALB/c mice with pSer₄-RBD (SEQ ID NO: 120-RBD), RBD-pSer₄ (RBD-SEQ ID NO: 120), or unmodified RBD combined with alum and boosted at 6 weeks. Consistent with prior reports (10, 11), RBD:alum immunization elicited weak IgG responses, with none of the animals seroconverting by 3 weeks post-prime at this dose; post-boost, weak IgG titers were detected that steadily declined over time (FIG. 2A). Both pSer-modified immunogens exhibited stronger serum responses, and the N-terminally modified RBD was particularly effective, with titers 57-fold greater than the control group at the peak of response 2 weeks post-boost (FIG. 2A-B). Further, traditional alum:RBD immunization elicited no detectable neutralizing responses even after boosting, while 3 of 5 animals receiving pSer₄-RBD (SEQ ID NO: 120-RBD):alum vaccination had pseudovirus (PSV) neutralizing titer ID50 (NT₅₀) >10³ post-boost (FIG. 2C-D). pSer₄-RBD (SEQ ID NO: 120) also significantly augmented the number of antibody-secreting cells in the bone marrow at day 112 (FIG. 2E). Altogether, these data suggest that pSer conjugation to the N-terminus of RBD can substantially enhance humoral responses to alum:RBD immunization.

TABLE 4
Antigen sequences

| Protein | Sequence |
|---|---|
| RBD | ITNLCPFGEVFNATRFASVYAWNRKRISNCVADYS |
| C-terminal | VLYNSASFSTFKCYGVSPTKLNDLCFTNVYADSFV |
| cysteine | IRGDEVRQIAPGQTGKIADYNYKLPDDFTGCVIAW |
| | NSNNLDSKVGGNYNYLYRLFRKSNLKPFERDISTE |
| | IYQAGSTPCNGVEGFNCYFPLQSYGFQPTNGVGYQ |
| | PYRVVVLSFELLHAPATVCGPKKSTNHHHHHHC |
| | (SEQ ID NO: 122) |
| RBD | CITNLCPFGEVFNATRFASVYAWNRKRISNCVADY |
| N-terminal | SVLYNSASFSTFKCYGVSPTKLNDLCFTNVYADSF |
| cysteine | VIRGDEVRQIAPGQTGKIADYNYKLPDDFTGCVIA |
| | WNSNNLDSKVGGNYNYLYRLFRKSNLKPFERDIST |
| | EIYQAGSTPCNGVEGFNCYFPLQSYGFQPTNGVGY |
| | QPYRVVVLSFELLHAPATVCGPKKSTNHHHHHH |
| | (SEQ ID NO: 123) |
| RBDJ | CITNLCPFGEVFNATRFASVYAWNRKRISNCVADY |
| N-terminal | SVLYNSASFSTFKCYGVSPTKLNDLCFTNVYADSF |
| cysteine | VIRGDEVRQIAPGQTGKIADYNYKLPDDFTGCVIA |
| sequences | WNSNNLDSKVGGNYNYKYRLFRKSNLKPFERDIST |
| | EIYQAGSTPCNGVEGFNCYWPLQSYGFQPTNGVGY |
| | QPYRVVVLSFELLHAPATVCGPKKSTN |
| | (SEQ ID NO: 124) |

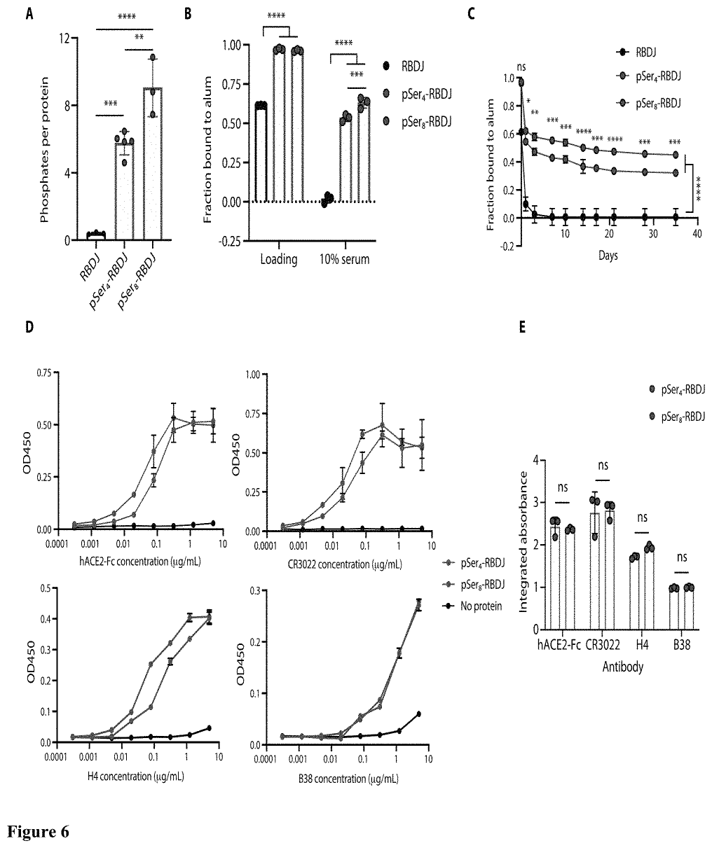

The amino acid sequences of RBD antigens used in these studies.
A Stabilized RBD Mutant Further Enhances the Immunogenicity of Alum:RBD Vaccines We recently developed a novel RBD variant containing two point mutations (L452K, F490W; table 4) engineered to improve manufacturability and stability of the antigen. This variant (hereafter, RBDJ) was also more immunogenic than Wuhan-Hu-1 RBD (hereafter, wild-type RBD) in mice (29). Given the inconsistent neutralizing responses observed in mice immunized with pSer₄-RBD (SEQ ID NO: 120-RBD), we thus tested whether RBDJ would benefit from pSer-tagging and assessed whether increasing the valency of the pSer tag could further enhance antibody responses. To this end, RBDJ N-terminally modified with a pSer₄ or pSer₈ (SEQ ID NO: 120 or 121) tag was synthesized (FIG. 6A). Both pSer₄-RBDJ and pSer₈-RBDJ (SEQ ID NO: 120-RBDJ & SEQ ID NO: 121-RBDJ) adsorbed efficiently to alum, and pSer₈-RBDJ (SEQ ID NO: 121-RBDJ) showed slightly higher retention on alum over time on exposure to serum/phosphate buffer (FIG. 6B-C). As observed with the wild-type RBD, pSer-RBDJ protein bound to alum particles retained robust binding to hACE2, CR3022, H4, and B38 (FIG. 6D-E).

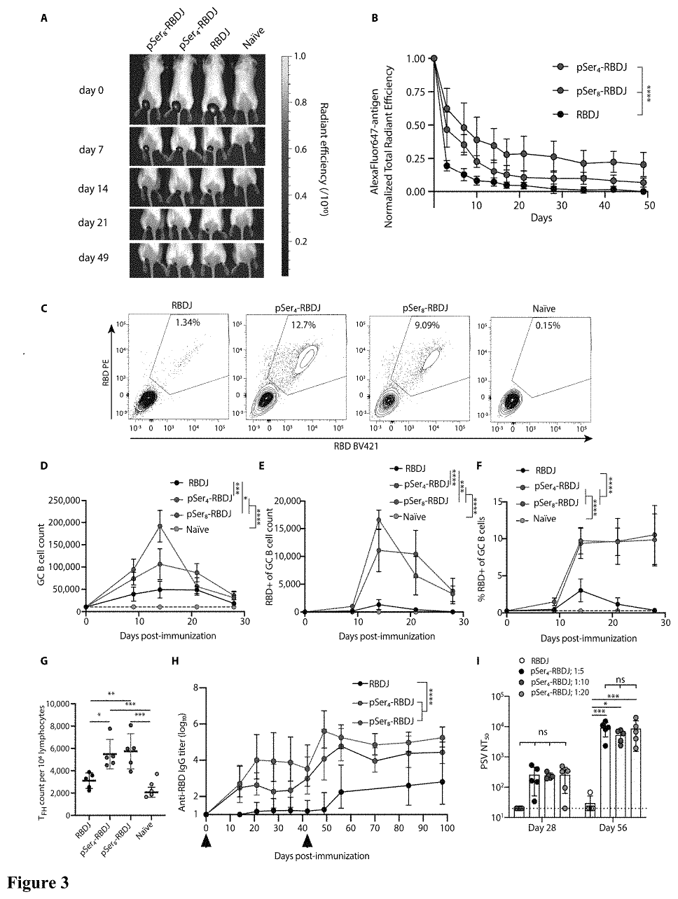

We previously found that pSer-modified HIV Env proteins trafficked to lymph nodes bound to alum particles, such that antigen-specific B cells directly internalized antigen-decorated alum particles (26). To gain insight into the behavior of pSer-tagged RBDJ and assess whether increased pSer valency impacted antigen availability kinetics in vivo, we fluorescently labeled the pSer-RBDJ proteins with an AlexaFluor™647 dye. Mice were immunized subcutaneously (s.c.) with these labeled vaccines near the tail base, and the kinetics of antigen clearance from the injection site over time were tracked by whole animal fluorescence imaging (FIG. 3A). Fluorescence from the RBD antigen steadily cleared from the immunization site, with the rate of decay ordered as RBDJ>pSer₄-RBDJ (SEQ ID NO: 120-RBDJ) >pSer₈-RBDJ (SEQ ID NO: 121-RBDJ) (FIG. 3B).

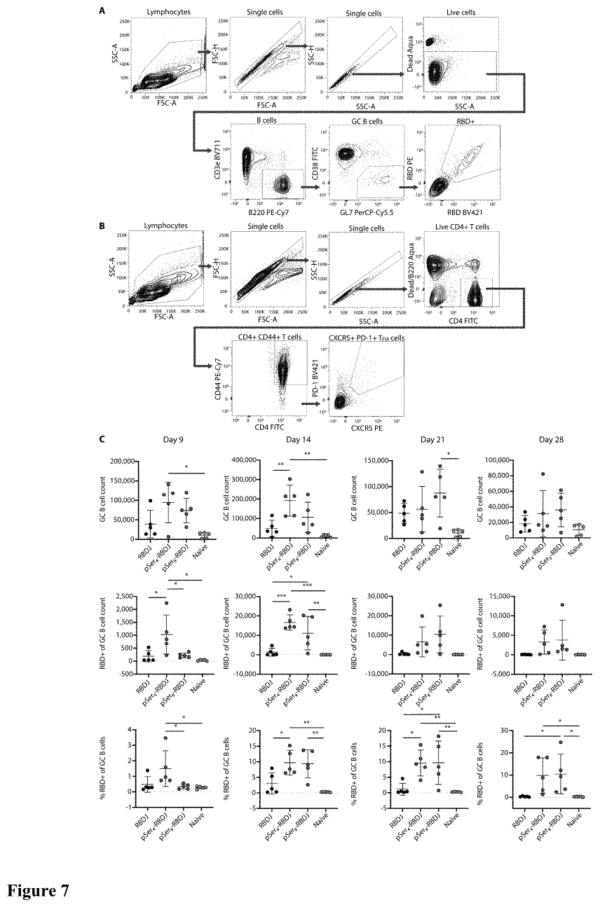

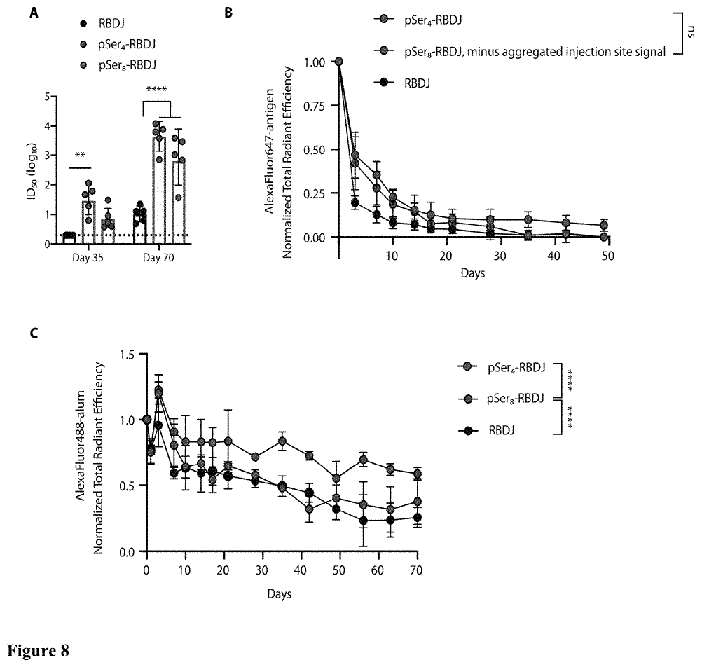

To determine whether these distinct vaccine kinetics impacted the immune response, we first quantified GC responses following alum:RBDJ immunization. Flow cytometry analysis of draining inguinal lymph nodes (dLNs) harvested at staggered time points post-injection revealed that phosphoserine-tagged RBDs elicited notably stronger GC responses than traditional alum:RBDJ immunization (FIG. 3C-F, FIG. 7A-C). The total GC response peaked at day 14, with pSer₄-RBDJ (SEQ ID NO: 120-RBDJ) eliciting the strongest response (FIG. 3D and FIG. 7C). Even more striking was the impact on antigen-specific GC B cells: both pSer₄-RBDJ (SEQ ID NO: 120-RBDJ) and pSer₈-RBDJ (SEQ ID NO: 121-RBDJ) primed a substantial population of RBD-specific GC B cells, while antigen-specific cells were very low in the alum:RBDJ control group across the entire time course (FIG. 3C, FIGS. 3E-F, and FIG. 7C). In addition, pSer-conjugated RBDJ elicited ˜2-fold greater T follicular helper cell (T_{FH}) responses than the unmodified immunogen (FIG. 3G). These enhanced GC and T_{FH} responses correlated with greatly increased IgG antibody responses for mice immunized with pSer-RBDJ compared to RBDJ (FIG. 3H), and these antibodies exhibited a significantly higher ability to block hACE2-RBD interactions (FIGS. 8A, 3I). Intriguingly, total binding IgG ELISA titers and hACE2 binding inhibition trended to be higher with pSer₄-RBDJ (SEQ ID NO: 120-RBDJ) vs. pSer₈-RBDJ (SEQ ID NO: 121-RBDJ), but these differences did not reach statistical significance. Notably, the antigen drainage characterization (FIG. 3A-B) suggested a portion of pSer₈-RBDJ (SEQ ID NO: 121-RBDJ) may be irreversibly trapped at the injection site. If we subtracted the plateau fluorescence signal from the total fluorescence of the pSer₈-RBDJ (SEQ ID NO: 121-RBDJ) group over time, the resulting “bioavailable” pSer₈-RBDJ (SEQ ID NO: 121-RBDJ) trajectory looks very similar to that of pSer₄-RBDJ (SEQ ID NO: 120-RBDJ) (FIG. 8B). Further, longitudinal tracking of alum drainage from the injection site revealed that ˜58% of alum remains at the injection site 70 days post-immunization for pSer₈-RBDJ (SEQ ID NO: 121-RBDJ) compared to ˜37% for pSer₄-RBDJ (SEQ ID NO: 120-RBDJ) (FIG. 8C). We hypothesize that these altered kinetics for antigen and alum clearance observed with the longer pSer₈-tagged (SEQ ID NO: 121-tagged) immunogen may reflect some level of inter-alum particle crosslinking mediated by the longer peptide tag, which inhibits disaggregation of alum particles and promotes their phagocytosis locally at the injection site, limiting delivery to the dLNs. Importantly, pSer₄-RBDJ (SEQ ID NO: 120-RBDJ) elicited peak serum IgG titers ˜4-fold greater than the same dose of pSer₄-RBD (SEQ ID NO: 120-RBDJ) with more consistent seroconversion (FIG. 2A and FIG. 311). Altogether, these data indicate that pSer-RBDJ vaccines elicit greatly enhanced GC and serum antibody responses compared to traditional immunization with admixed RBDJ and alum.

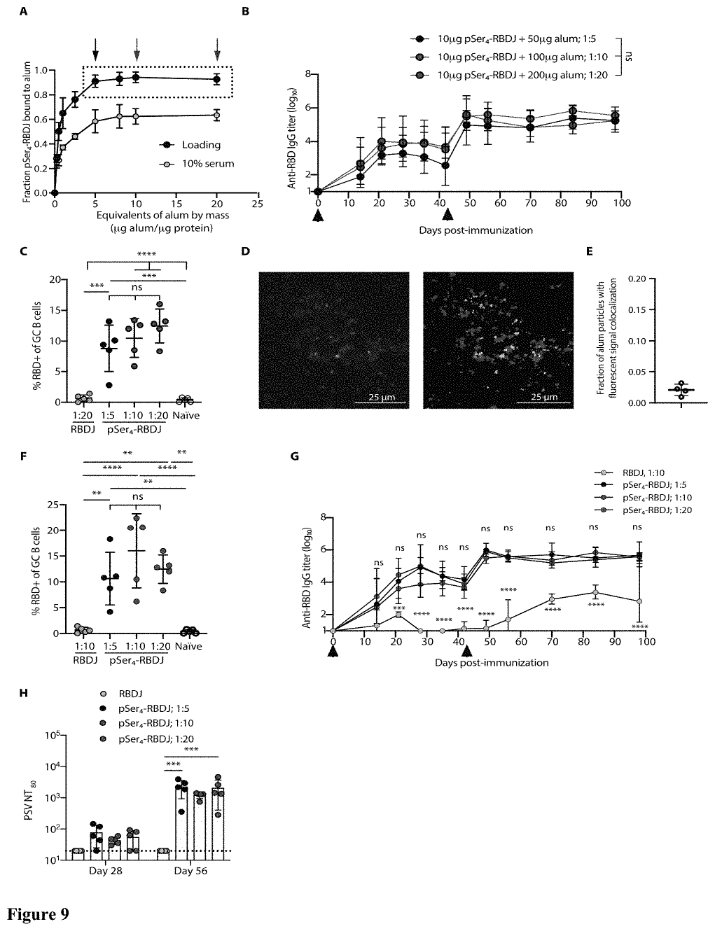

We also investigated the impact of alum dose and antigen density on humoral immune responses. Varying the amount of RBDJ added to a fixed amount of alum, we identified a range of antigen densities for which there was comparable pSer₄-RBDJ (SEQ ID NO: 120-RBDJ) loading and retention on alum (FIG. 9A). Based on the reported surface area of Alhydrogel alum (30), antigen:alum mass ratios of 1:5, 1:10, and 1:20 correspond to an estimated average spacing between RBDs on alum particles of 9.6 nm, 13.5 nm, and 19.2 nm, respectively. To determine whether antigen density variation in this range affects the immune response, mice were immunized with a constant dose of pSer₄-RBDJ (SEQ ID NO: 120-RBDJ) loaded on varying quantities of alum (50, 100, or 200 μg), and the serum antibody responses were tracked longitudinally. Interestingly, differences between these 3 groups were very modest and not statistically significant (FIG. 9B). Examining GC responses 14 days post-immunization, antigen-specific GC B cell frequencies showed a slight trend toward increased responses at lower antigen density/higher alum dose, but these differences again were not significant (FIG. 9C). These experiments are potentially confounded by the convolution of antigen density with alum amount, and thus we also devised a second experimental approach: immunizations were prepared by first loading antigen on alum at the specified ratios, and then supplementing in extra alum just prior to immunization to bring the alum dose to 200 μg for all mice. To confirm that pSer₄-RBDJ (SEQ ID NO: 120-RBDJ) would not redistribute when additional alum was added, we imaged alum particles loaded with fluorophore-tagged pSer₄-RBDJ (SEQ ID NO: 120-RBDJ) that were mixed with RBD-free alum tagged by a low density of pSer₄-Alexa™ (SEQ ID NO: 120-Alexa™) dye and incubated together for 2 days. As shown in FIG. 9D-E, no transfer of pSer₄-RBDJ (SEQ ID NO: 120-RBDJ) (red) to the bare alum particles (cyan) was observed when the mixture was examined by microscopy. Therefore, using this approach we repeated immunizations varying the antigen density across the same RBD:alum mass ratios and assessed GC responses, serum IgG over time, and neutralizing antibody titers. Similar to the previous experiments, GC responses were not statistically different between the groups (FIG. 9F). There was a transient enhancement in humoral responses post-prime with increasing antigen density, but all groups responded similarly post-boost, and PSV NT₅₀ were not different across the three pSer₄-RBDJ groups (SEQ ID NO: 120-RBDJ) (FIG. 9G-H, FIG. 3I). The neutralizing responses elicited by pSer₄-RBDJ (SEQ ID NO: 120-RBDJ):alum, however, were significantly higher than unmodified RBDJ and were notably more consistent than we observed with wild-type pSer₄-RBD (SEQ ID NO: 120-RBD):alum, with all animals primed to produce high levels of neutralizing responses at a mean PSV NT₅₀ of ˜5,270 two weeks post-boost (n.b., compare 1:10 antigen density in FIG. 3I with FIG. 2C). Thus, pSer₄ (SEQ ID NO: 120) modification of RBDJ enhanced GC and neutralizing antibody responses, but these responses were not sensitive to the density of antigen loading on alum.

### Conjugation of Antigen and Adjuvants to Alum Synergistically Amplifies Humoral Responses

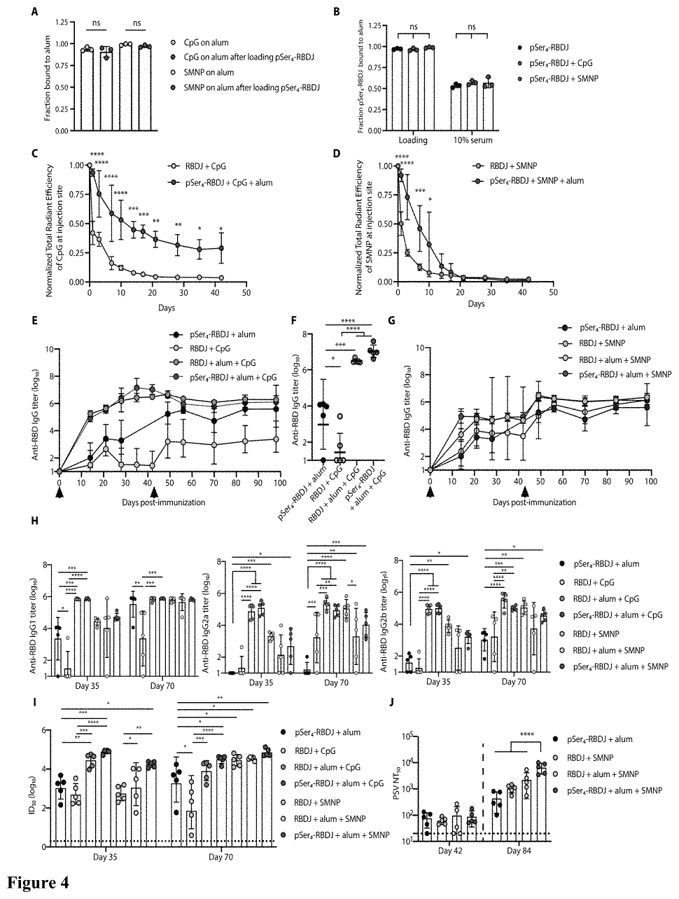

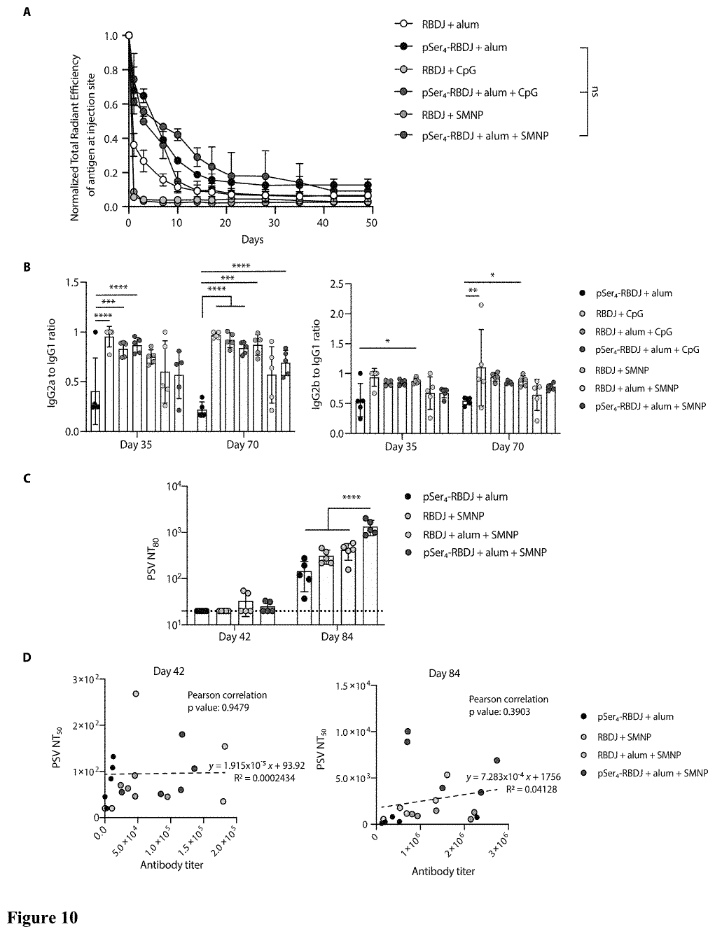

Although pSer anchoring RBD to alum greatly enhanced its immunogenicity, alum remains an adjuvant with modest potency in large animals and humans. We hypothesized that combining alum with a molecular co-adjuvant employing the same ligand exchange reaction used to anchor RBD immunogen would synergistically enhance the immune response, by prolonging the exposure of dLNs to both antigen and inflammatory cues. We thus tested the behavior of two clinically relevant phosphate-containing co-adjuvants, CpG, a single-stranded DNA TLR9 agonist containing phosphorothioates in the oligonucleotide backbone, and SMNP (27), an ISCOMs-like ˜40 nm diameter nanoparticle formed by the self-assembly of phospholipids, saponin, and the TLR4 agonist monophosphoryl lipid A, which binds to alum via phosphate groups of the lipids and MPLA, in combination with pSer₄-RBDJ (SEQ ID NO: 120-RBDJ) and alum. Both CpG and SMNP demonstrated strong alum adsorption and retention on alum (3:10 and 1:20 mass ratios, respectively) in the presence of mouse serum, suggesting strong ligand exchange-mediated binding (FIG. 4A). We sequentially added pSer₄-RBDJ (SEQ ID NO: 120-RBDJ) and the two co-adjuvants to alum; importantly, we found that neither CpG nor SMNP displaced bound pSer₄-RBDJ (SEQ ID NO: 120-RBDJ) (FIG. 4B). Whole animal fluorescence imaging of the injection site following immunization with labeled CpG or SMNP adsorbed to alum together with pSer₄-RBD (SEQ ID NO: 120-RBD) revealed sustained drainage of the co-adjuvants compared to injection of these adjuvants in the absence of alum (FIG. 4C-D). Notably, addition of these co-adjuvants did not disrupt the sustained drainage of pSer₄-RBDJ (SEQ ID NO: 120-RBDJ) when loaded on alum: when CpG or SMNP were adsorbed to alum with labeled pSer₄-RBDJ (SEQ ID NO: 120-RBDJ), there was no significant difference in the kinetics of antigen clearance when compared to pSer₄-RBDJ (SEQ ID NO: 120-RBDJ) on alum alone (FIG. 10A).

In order to investigate the impact of these alum-bound co-adjuvants on humoral responses, we immunized mice with combinations of CpG or SMNP bound to alum with RBDJ or pSer₄-RBDJ (SEQ ID NO: 120-RBDJ) and tracked serum antibody responses over time. Notably, the addition of CpG to pSer₄-RBDJ (SEQ ID NO: 120-RBDJ):alum or RBDJ:alum immunizations dramatically enhanced IgG antibody titers compared to pSer₄-RBDJ (SEQ ID NO: 120-RBDJ):alum or soluble RBDJ plus CpG following the priming immunization (FIG. 4E, F). There were also trends of increased IgG antibody titers for alum-bound antigen and co-adjuvant SMNP (FIG. 4G). Examination of individual IgG isotypes showed that IgG1, IgG2a, and IgG2b titers were all substantially increased when pSer-RBDJ:alum was combined with each of the co-adjuvants (FIG. 411), and the IgG2a/IgG1 and IgG2b/IgG1 ratios were increased with the addition of the co-adjuvants (FIG. 10B). The addition of CpG and SMNP to pSer:alum immunizations also elicited more functional antibody responses, as serum from immunized mice demonstrated stronger inhibition of hACE2-RBD binding both post-prime and post-boost (FIG. 4I). Notably, maximal hACE2 binding inhibition/neutralizing responses required that alum was combined with one of the co-adjuvants and that the RBD was pSer-modified. This finding was even more starkly illustrated by pseudovirus neutralizing antibody titers measured for animals immunized with pSer-RBDJ:alum±SMNP: immunization with pSer₄-RBDJ (SEQ ID NO: 120-RBDJ):alum or RBDJ+SMNP elicited PSV NT₅₀ titers ˜10-fold weaker than the pSer₄-RBDJ (SEQ ID NO: 120-RBDJ):alum:SMNP combination (FIG. 4J, FIG. 10C). Notably, there was no statistically significant correlation between serum IgG ELISA binding titers and PSV NT₅₀ at day 42 or 84 (FIG. 10D), suggesting that the serum IgG ELISA binding titers is not predictive of neutralizing titers for these groups.

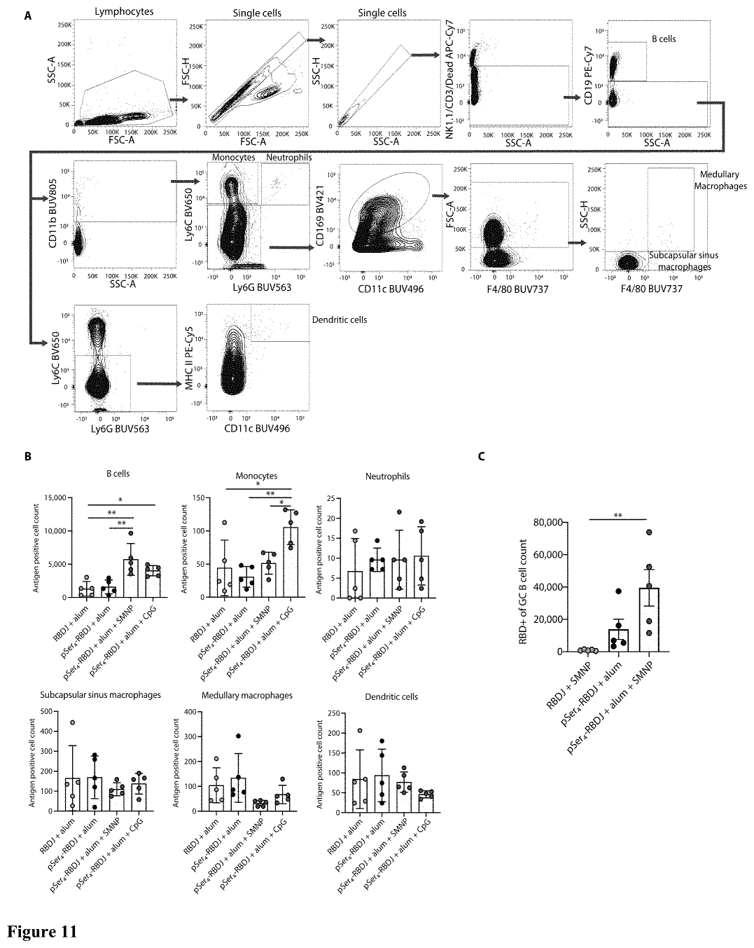

To further investigate the basis of this enhanced neutralizing antibody response, we investigated the impact of CpG or SMNP co-adjuvants on the cellular localization of antigen. Mice were immunized with AlexaFluor™-labeled RBDJ, and the number of cells positive for antigen was assessed among B cells, monocytes, neutrophils, subcapsular sinus macrophages, medullary macrophages, and dendritic cells (FIG. 11A-B). B cells showed a significant increase in antigen uptake following pSer₄-RBDJ (SEQ ID NO: 120-RBDJ):alum+SMNP immunization compared to RBDJ:alum and pSer₄-RBDJ (SEQ ID NO: 120-RBDJ):alum, whereas there was a significant increase in monocyte uptake of antigen for pSer₄-RBDJ (SEQ ID NO: 120-RBDJ):alum+CpG compared to RBDJ:alum and pSer₄-RBDJ (SEQ ID NO: 120-RBDJ):alum. These differences in antigen distribution may contribute to the synergistic benefit of co-adjuvants with pSer₄-RBDJ (SEQ ID NO: 120-RBDJ):alum. To elucidate the basis of the enhanced neutralization responses in mice immunized with pSer₄-RBDJ (SEQ ID NO: 120-RBDJ):alum+SMNP despite comparable overall IgG titers with RBDJ+SMNP, we immunized mice with unmodified RBDJ or pSer₄-RBDJ (SEQ ID NO: 120-RBDJ) and alum and/or SMNP and assessed the RBD-specific GC B cell responses in the dLNs at day 14 post-immunization. Notably, pSer₄-RBDJ (SEQ ID NO: 120-RBDJ):alum+SMNP elicited significantly higher RBD-specific GC B cell responses compared to RBDJ+SMNP (FIG. 11C). These data suggest that stronger neutralizing responses observed in mice immunized with pSer₄-RBDJ (SEQ ID NO: 120-RBDJ):alum+SMNP compared to RBDJ+SMNP are driven by more robust antigen-specific GC responses when alum anchoring and SMNP are combined. Hence, co-conjugation of molecular adjuvants and the immunogen with alum synergistically amplifies humoral immunity to RBD.

### Discussion

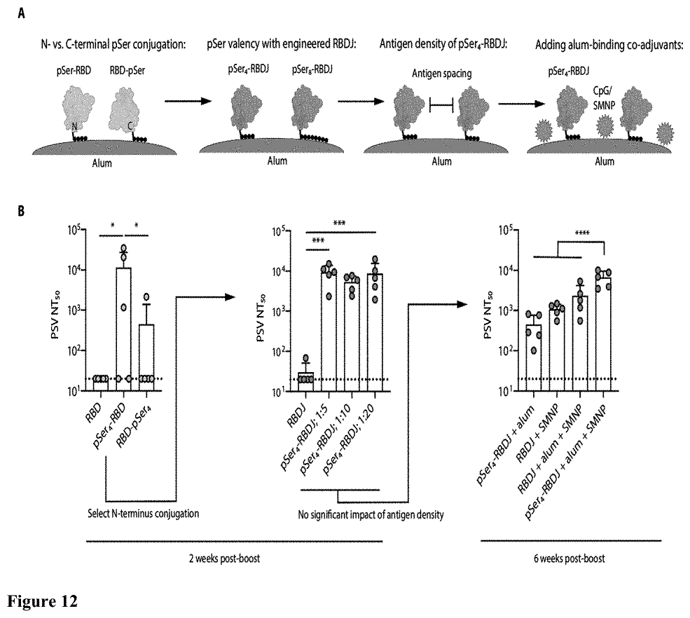

There is a need for additional safe and effective SARS-CoV-2 vaccines to facilitate global vaccine coverage. Given the emergence of novel SARS-CoV-2 variants, it is especially important that these vaccines elicit responses that retain activity against circulating variants of concern. Subunit vaccines are an attractive approach to achieve global coverage, as they can be rapidly scaled for manufacturing, and their distribution does not require ultra-cold storage temperatures. Here we describe an approach using alum, a low-cost adjuvant with widespread clinical use, that elicited potent humoral immune responses and neutralization against SARS-CoV-2. By modifying the RBD antigen with a short peptide linker, the duration of antigen drainage from the injection site was substantially extended, leading to strong antigen-specific GC responses which lasted over a month post-immunization. Through the optimization of this immunization platform, testing the impact of N- versus C-terminal pSer conjugation, pSer valency, antigen density, and the addition of alum-binding co-adjuvants, the platform achieved continually higher and more consistent antibody and neutralization responses in mice (FIG. 12). Notably, the addition of CpG or SMNP co-adjuvants to pSer-RBD plus alum immunizations also promoted a more balanced Th1/Th2 bias to the antibody response.

The pSer modification approach employed here provides a simple and robust strategy to prolong antigen availability in a clinically translatable vaccine regimen. The alum-anchoring strategy used here has the additional capacity to help potentiate B cell responses by presenting many copies of antigen bound to a single alum particle, promoting BCR crosslinking and early signaling/B cell activation (26). However, in the case of RBD, varying antigen density did not impact any of the measures of the humoral responses assessed here, suggesting either that the RBD densities explored here did not cover a wide enough range to detect an effect on B cell triggering and/or that some release of pSer-RBD from alum particles occurs over time, thus diluting the “alum presentation” effect.

Studies applying repeated injections to achieve extended dosing in cancer vaccines have demonstrated the importance of sustained exposure to both antigen and inflammatory cues in peptide vaccines for optimal T cell responses (39), but the role of extended adjuvant exposure on humoral immunity is not well understood. To couple the kinetics of antigen and adjuvant delivery to lymph nodes, we tested here the use of two different molecular adjuvants, CpG and a nanoparticle-formulated saponin, each of which could undergo the same type of ligand exchange reaction with alum as employed in our pSer-modified immunogens. With each of these co-adjuvants, we observed sustained release from the injection site in the presence of alum. These altered vaccine kinetics correlated with enhanced antibody responses and neutralization that were much more than additive over the individual responses elicited by alum or the co-adjuvants in isolation, suggesting strong synergy induced by alum binding. We hypothesize that altered delivery kinetics achieved by ligand exchange binding to alum play an important role in the potency of this adjuvant combination.

As a platform, this technology promotes sustained antigen and co-adjuvant drainage from the injection site, inducing potent humoral immune responses against SARS-CoV-2 using alum, a low-cost adjuvant with widespread clinical use. In the context of more immunogenic antigens, this platform could also be beneficial to promote a dose-sparing strategy to increase vaccine availability. Our findings demonstrate that combinations of adjuvants can enable new immunological mechanisms of action, providing vaccine formulations with activity greater than the individual components, and enhance the potency of subunit vaccine antigens.

Our results demonstrate that:
1. Phosphoserine peptide modification facilitates stable binding of SARS-CoV-2 RBD to alum. We first tested whether coupling a pSer peptide tag to Wuhan-Hu-1 SARS-CoV-2 RBD could engender stable binding to alum without disrupting key epitopes on the antigen. RBD (amino acids 332-532 of SARS-CoV-2 S protein) modified with a histag for purification and containing an N- or C-terminal free cysteine was expressed in K. phaffii, and then conjugated with a peptide tag containing a maleimide group linked to a 6-unit poly(ethylene glycol) spacer followed by four phosphoserine residues. Measurement of the mean number of phosphates per protein using a malachite green assay revealed that the N-terminal-coupled pSer₄-RBD (SEQ ID NO: 120-RBD) and C-terminal-modified RBD-pSer₄ (RBD-SEQ ID NO: 120) gained the expected ˜4 phosphates per protein (FIG. 1A). Incubation of unmodified RBD with alum in tris-buffered saline led to adsorption of only ˜25% of the antigen (FIG. 1B “loading”), most of which desorbed following a 24-hour incubation of the alum in phosphate buffer containing 10% serum (FIG. 1B “10% serum”). By contrast, both pSer-RBDs exhibited high levels of alum binding in buffer, with the majority remaining bound after serum/phosphate exposure (FIG. 1B). To confirm that the alum-bound RBD conjugates were structurally intact relative to unmodified protein, we used a modified sandwich ELISA approach to probe the antigenicity of the constructs. Unmodified RBD was captured on plates coated with a histag-specific antibody, while pSer-conjugated RBD was captured on alum-coated plates (FIG. 1C). The immobilized RBD was then probed for binding to serial dilutions of recombinant hACE2 protein, the target receptor recognized by RBD, or monoclonal antibodies CR3022 (which recognizes a highly conserved epitope distal from the receptor binding site), H4, or B38. As shown in FIGS. 1D-H, the pSer-modified RBDs had antigenicity profiles indistinguishable from unmodified RBD, and the proteins captured on alum retained recognition of both probes. Thus, pSer modification allowed substantially enhanced RBD binding to alum without disrupting its structure.
2. N-terminal pSer modification enhances the immunogenicity of RBD antigens. We immunized BALB/c mice with pSer₄-RBD (SEQ ID NO: 120-RBD), RBD-pSer₄(RBD-SEQ ID NO: 120), or unmodified RBD combined with alum and boosted at 6 weeks.

RBD:alum immunization elicited weak IgG responses, with none of the animals seroconverting by 3 weeks post-prime at this dose; post-boost, weak IgG titers were detected that steadily declined over time (FIG. 2A). Both pSer-modified immunogens exhibited stronger serum responses, and the N-terminally modified RBD was particularly effective, with titers 57-fold greater than the control group at the peak of response 2 weeks post-boost (FIG. 2A-B). Further, traditional alum:RBD immunization elicited no detectable neutralizing responses even after boosting, while 3 of 5 animals receiving pSer₄-RBD (SEQ ID NO: 120-RBD):alum vaccination had pseudovirus (PSV) neutralizing titer ID50 (NT₅₀)>10³ post-boost (FIG. 2C-D). pSer₄-RBD (SEQ ID NO: 120-RBD) also significantly augmented the number of antibody-secreting cells in the bone marrow at day 112 (FIG. 2E). Altogether, these data suggest that pSer conjugation to the N-terminus of RBD can substantially enhance humoral responses to alum:RBD immunization.
3. A stabilized RBD mutant further enhances the immunogenicity of alum:RBD vaccines. We recently developed a novel RBD variant containing two point mutations (L452K, F490W) engineered to improve manufacturability and stability of the antigen. This variant (hereafter, RBDJ) was also more immunogenic than Wuhan-Hu-1 RBD (hereafter, wild-type RBD) in mice. Given the inconsistent neutralizing responses observed in mice immunized with pSer₄-RBD (SEQ ID NO: 120-RBD), we thus tested whether RBDJ would benefit from pSer-tagging and assessed whether increasing the valency of the pSer tag could further enhance antibody responses. To this end, RBDJ N-terminally modified with a pSer₄ or pSer₈ (SEQ ID NO: 120 or 121) tag was synthesized. To gain insight into the behavior of pSer-tagged RBDJ and assess whether increased pSer valency impacted antigen availability kinetics in vivo, we fluorescently labeled the pSer-RBDJ proteins with an AlexaFluor™647 dye. Mice were immunized subcutaneously (s.c.) with these labeled vaccines near the tail base, and the kinetics of antigen clearance from the injection site over time were tracked by whole animal fluorescence imaging (FIG. 3A). Fluorescence from the RBD antigen steadily cleared from the immunization site, with the rate of decay ordered as RBDJ>pSer₄-RBDJ (SEQ ID NO: 120-RBDJ)>pSer₈-RBDJ (SEQ ID NO: 121-RBDJ) (FIG. 3B). To determine whether these distinct vaccine kinetics impacted the immune response, we first quantified GC responses following alum:RBDJ immunization. Flow cytometry analysis of draining inguinal lymph nodes (dLNs) harvested at staggered time points post-injection revealed that phosphoserine-tagged RBDs elicited notably stronger GC responses than traditional alum:RBDJ immunization (FIG. 3C-F). The total GC response peaked at day 14, with pSer₄-RBDJ (SEQ ID NO: 120-RBDJ) eliciting the strongest response (FIG. 3D). Even more striking was the impact on antigen-specific GC B cells: both pSer₄-RBDJ (SEQ ID NO: 120-RBDJ) and pSer₈-RBDJ (SEQ ID NO: 121-RBDJ) primed a substantial population of RBD-specific GC B cells, while antigen-specific cells were very low in the alum:RBDJ control group across the entire time course (FIG. 3C, FIG. 3E-F). In addition, pSer-conjugated RBDJ elicited ˜2-fold greater T follicular helper cell (T_{FH}) responses than the unmodified immunogen (FIG. 3G). These enhanced GC and T_{FH} responses correlated with greatly increased IgG antibody responses for mice immunized with pSer-RBDJ compared to RBDJ (FIG. 311). Importantly, pSer₄-RBDJ (SEQ ID NO: 120-RBDJ) elicited peak serum IgG titers ˜4-fold greater than the same dose of pSer₄-RBD (SEQ ID NO: 120-RBD) with more consistent seroconversion (FIG. 2A and FIG. 311). Altogether, these data indicate that pSer-RBDJ vaccines elicit greatly enhanced GC and serum antibody responses compared to traditional immunization with admixed RBDJ and alum.
4. We hypothesized that combining alum with a molecular co-adjuvant employing the same ligand exchange reaction used to anchor RBD immunogen would synergistically enhance the immune response, by prolonging the exposure of dLNs to both antigen and inflammatory cues. We thus tested the behavior of SMNP adjuvant. SMNP demonstrates strong alum adsorption and retention on alum in the presence of mouse serum, indicating strong ligand exchange-mediated binding (FIG. 4A). This alum binding behavior translated to sustained drainage of SMNP from the injection site in vivo. Whole animal fluorescence imaging of the injection site following immunization with labeled SMNP adsorbed to alum revealed sustained drainage of SMNP compared to injection in the absence of alum (FIG. 4D). This demonstrated that anchoring of SMNP to alum significantly prolonged the persistence of SMNP in vivo.
5. The combination of alum and SMNP (alum:SMNP) synergistically enhances immune responses to immunization. To investigate the impact of the alum-bound co-adjuvant on humoral responses, we immunized mice with a SARS-CoV-2 antigen, receptor binding domain (RBD) of the S protein, with or without conjugated pSer, with alum alone, SMNP alone, or alum:SMNIP and tracked serum antibody responses over time. There were trends of increased IgG antibody titers for alum bound antigen and co-adjuvant SMNP (FIG. 4G). Examination of individual IgG isotypes showed that IgG1, IgG2a, and IgG2b titers were all substantially increased when pSer-RBD:alum was combined with SMNIP (FIG. 411). The addition of SMNP to pSer:alum immunizations also elicited more functional antibody responses, as serum from immunized mice demonstrated stronger inhibition of hACE2-RBD binding both post-prime and post-boost (FIG. 4I). Notably, maximal hACE2 binding inhibition/neutralizing titers required that alum was combined with SMNP and that the RBD was pSer-modified. This finding was even more starkly illustrated by pseudovirus neutralization titers measured for animals immunized with pSer-RBD:alum ±SMNP: immunization with pSer-RBD:alum or RBD+SMNP elicited neutralizing titers ˜10 fold weaker than the pSer-RBD:alum:SMNP combination (FIG. 4J). Hence, co-conjugation of SMNP and the antigen with alum synergistically amplifies humoral immunity to RBD.

### Materials and Methods

Phosphoserine Peptide Synthesis pSer peptide linkers were synthesized using solid phase synthesis on low-loading TentaGel Rink Amide resin (0.2 meq/g, Peptides International, catalog no. R28023) as described previously (26). Briefly, resin was deprotected with 20% piperidine (Sigma Aldrich, catalog no. 411027) in dimethylformamide (DMF, Sigma Aldrich, catalog no. 319937-4L), and peptide couplings were performed with 4 equivalents of Fmoc-Ser(PO(OBzl)OH)-OH (Millipore Sigma, catalog no. 8520690005) and 3.95 equivalents of hexafluorophosphate azabenzotriazole tetramethyl uranium (HATU, Millipore Sigma catalog no. 148893-10-1) for 2 hours at 25° C. pSer residues were deprotected with 5% 1,8-diazabicyclo[5.4.0]undec-7-ene (DBU, Sigma Aldrich, catalog no. 139009) in DMF. Double couplings were performed after the third residue. An Fmoc-protected 6-unit oligoethylene glycol linker (Peptides International, catalog no. DPG-5750) was then coupled to the peptide and subsequently deprotected and reacted with N-maleoyl-β-alanine (Sigma Aldrich, catalog no. 394815). Completion of each deprotection and coupling step was confirmed by a ninhydrin test (Sigma Aldrich, catalog no. 60017). pSer side chains were deprotected and the peptide was cleaved from the resin in 95% trifluoroacetic acid (Sigma Aldrich, catalog no. T6508), 2.5% H₂0, and 2.5% triisopropylsilane (Sigma Aldrich, catalog no. 233781), for 2.5 hours at 25° C. The product was precipitated in 4° C. diethyl ether (Sigma Aldrich, catalog no. 673811) and dried under N2, then purified by HPLC on a C18 column (Agilent Zorbax 300SB-C18) using 0.1 M triethylammonium acetate buffer (Glen Research, catalog no. 60-4110-62) in an acetonitrile gradient. The peptide mass was confirmed by matrix-assisted laser desorption/ionization-time of flight mass spectrometry. For imaging experiments, the pSer₄-AlexaFluor™488 (SEQ ID NO: 120—AlexaFluor™488) conjugate was synthesized as described for the pSer component of the linker, followed by deprotection and coupling to Fmoc-5-azido-pentanoic acid (Anaspec, catalog no. AS-65518-1). The peptide was deprotected with 20% piperidine in dimethylformamide prior to cleavage from the resin in 95% trifluoroacetic acid, 2.5% H₂0, and 2.5% triisopropylsilane for 2.5 hours at 25° C. The product was then precipitated in 4° C. diethyl ether, and dried under N2, and purified by HPLC on a C18 column using 0.1M triethylammonium acetate buffer in an acetonitrile gradient. The peptide mass was confirmed by matrix-assisted laser desorption/ionization-time of flight mass spectrometry. This pSer₄-azide (SEQ ID NO: 120-azide) linker was reacted with one equivalent of AlexaFluor™488-DBCO (Click Chemistry Tools, catalog no. 1278) overnight at 4° C. in a Cu-free click reaction in PBS (pH 7.2-7.4) and subsequently purified by HPLC on a C18 column using 0.1M triethylammonium acetate buffer in an acetonitrile gradient.

Antigen Production and pSer Conjugation

RBD immunogens were expressed in yeast strains derived from Komagataella phaffii (NRRL Y-11430) as described previously (29). Protein was purified using the InSCyT purification module as described previously (58). Columns were equilibrated in buffer prior to each run. His-tagged RBDs were purified with a 1 ml HisTrap HP column (Cytiva Life Sciences, catalog no. 29051021) on an AKTA pure 25 L FPLC system (Cytiva Life Sciences, catalog no. 29018224). The column was equilibrated with a binding buffer composed of 25 mM imidazole, 25 mM sodium phosphate, 500 mM NaCl, pH 7.4. Protein-containing supernatant was applied to the column via a S9 sample pump (Cytiva Life Sciences, catalog no. 29027745) at a rate of 2 ml/min. After washing the column with binding buffer, the his-tagged RBD (amino acids 332-532 of SARS-CoV-2 Wuhan-Hu-1 S protein; GenBank: MN908947.3) was eluted with 500 mM imidazole, 25 mM sodium phosphate, 500 mM NaCl, pH 7.4. For non-histagged RBDs, protein-containing supernatant was adjusted to pH 4.5 using 100 mM citric acid and subsequently loaded into a pre-packed 5 ml CMM HyperCel column (Pall Corporation, catalog no. PRCCMMHCEL5ML), re-equilibrated with 20 mM sodium citrate pH 5.0, washed with 20 mM sodium phosphate pH 5.8, and eluted with 20 mM sodium phosphate pH 8.0, 150 mM NaCl. Eluate from column 1 above 15 mAU was flowed through a 1 ml pre-packed HyperCel STAR AX column (Pall Corporation, catalog no. PRCSTARAX1ML). Flow-through from column 2 above 15 mAU was collected.

Antigens expressed with a free terminal cysteine were reduced at 1 mg/ml with 2 molar equivalents of tris(2-carboxyethyl)phosphine (TCEP, ThermoFisher, catalog no. 20490) and incubated at 25° C. for 10 minutes. TCEP was subsequently removed from reduced protein solutions using Amicon Ultra Centrifugal Filters (10 kDa MWCO, Millipore Sigma, catalog no. UFC501096) in tris-buffered saline (TBS, Sigma Aldrich, catalog no. T5912), and 1 mg/ml antigen was reacted with 2 molar equivalents of pSer-maleimide linkers for 16 hours at 4° C. in TBS (pH 7.2-7.4). Free pSer linker was subsequently removed using centrifugal filters in TBS, and pSer-antigen was buffer exchanged to PBS. The pSer₄-cytochrome C (SEQ ID NO: 120-cytochrome C) used for antigenicity profiling of immunogens was prepared as described, using cytochrome C from Saccharomyces cerevisiae (Sigma Aldrich, catalog no. C2436). The number of pSer residues conjugated to the antigen was assessed using the Malachite Green Phosphoprotein Phosphate Estimation Assay Kit (Thermo Scientific, catalog no. 23270) against a standard curve of pSer-maleimide linker. Signal from pSer-antigen was compared to the background from an unconjugated antigen control. Fluorescently labeled protein used in imaging experiments were prepared by reacting 1 mg/ml antigen in 50 mM sodium bicarbonate buffer for 1 hour at 25° C. with 6 molar equivalents of AlexaFluor™647 NHS ester (Invitrogen, catalog no. A20006) for alum binding studies and whole-mouse imaging or AlexaFluor™555 NHS ester (Invitrogen, catalog no. A20009) for microscopy experiments. Labeled antigen was purified by centrifugal filtration.

### SMNP Adjuvant Synthesis

Saponin-MPLA nanoparticles (SMNP) adjuvant was prepared as previously described (27). Briefly, solutions at 20 mg/ml were prepared of cholesterol (Avanti Polar Lipids, catalog no. 700000), DPPC (Avanti Polar Lipids, catalog no. 850355), and PHAD MPLA (Avanti Polar Lipids, catalog no. 699800P) in 20% MEGA-10 (Sigma, catalog no. D6277) detergent. Quil-A saponin (InvivoGen, catalog no. vac-quil) was dissolved in Milli-Q water at a final concentration of 100 mg/ml. These were mixed at a mass ratio of 10:2:1:1 (Quil-A:chol:DPPC:MPLA) and diluted in PBS to a final cholesterol concentration of 1 mg/ml. The solution was equilibrated overnight at 25° C. and then dialyzed against PBS using a 10 kDa MWCO cassette. The adjuvant was then sterile filtered, concentrated using Amicon Ultra Centrifugal Filters (50 kDa MWCO, Millipore Sigma, catalog no. UFC505096), and purified by FPLC using a Sephacryl S-500 HR size exclusion column. SMNP labeled with Cy7 was prepared as described incorporating 1,2-distearoyl-sn-glycero-3-phosphoethanolamine-N-(Cyanine 7) (Avanti Polar Lipids, catalog no. 810347) in place of 10 mol % of the MLPA.

### Antigen and Adjuvant Alum Binding and Release

AlexaFluor™647-labeled antigen was loaded onto Alhydrogel (alum, InvivoGen, catalog no. vac-alu-250) in TBS at a 1:10 antigen:alum mass ratio, unless otherwise specified, for 30 minutes on a tube rotator at 25° C. To assess antigen binding to alum, samples were immediately centrifuged at 10,000×g for 10 minutes to pellet alum, and the fluorescence of the supernatant was measured against a standard curve of labeled antigen. To assess the release of antigen from alum, mouse serum was added to antigen-alum solutions post-loading to a final mouse serum concentration of 10 vol % and incubated at 37° C. for 24 hours, unless otherwise specified. Samples were subsequently centrifuged at 10,000 g for 10 minutes to pellet alum, and the fraction of protein bound to alum was measured by fluorescence using a Tecan Infinite M200 Pro plate reader. Experiments investigating CpG binding and release from alum were performed using FITC-labeled CpG 1826 (InvivoGen, catalog no. tlrl-1826f) with a 3:10 CpG:alum mass ratio. Experiments investigating SMNP binding and release from alum were performed using Cy7-labeled SMNP with a 1:20 SMNP:alum mass ratio.

### Antigenicity Profiling of RBD Immunogens

Antigenicity profiling of antigens was completed by comparing antibody binding curves of pSer-conjugated RBD or RBDJ on alum against those of unmodified RBD or RBDJ. To capture alum on Nunc Maxisorp ELISA plates (Invitrogen, catalog no. 44-2404-21), plates were first coated with pSer₄-conjugated cytochrome C (SEQ ID NO: 120--cytochrome C) at 2 μg/ml for 4 hours at 25° C. Alum was then added at 200 μg/ml and captured by pSer₄-cytochrome C (SEQ ID NO: 120-cytochrome C) overnight at 4° C. To capture unmodified RBD, plates were coated with a rabbit anti-histag antibody (GenScript, catalog no. A00174-40) at 2 μg/ml overnight at 4° C. Plates were washed with 0.05% Tween-20 in PBS and incubated with 2 μg/ml protein in 2% BSA in PBS for 2 hours at 25° C. CR3022 monoclonal antibody (Abcam, catalog no. ab273073), hACE2-Fc chimera (InvivoGen, catalog no. fc-hace2), H4 (InvivoGen, catalog no. cov2rbdc1-mab1), or B38 (InvivoGen, catalog no. cov2rbdc2-mab1) was added at 5 μg/ml with 1:4 serial dilutions for 2 hours at 25° C. Plates were washed and antibody binding was detected with a goat anti-human HRP conjugated secondary antibody (BioRad, catalog no. 1721050) at 1:5000 dilution in PBS containing 2% BSA and then developed with 3,3′,5,5′-tetramethylbenzidine (ThermoFisher, catalog no. 34028), stopped with 2N sulfuric acid and immediately read (450 nm with 540 nm reference) on a BioTek Synergy2 plate reader.

### Animals and Immunizations

Experiments and handling of mice were conducted under federal, state and local guidelines under an Institutional Animal Care and Use Committee (IACUC) approved protocol. Female 6-8-week-old BALB/c mice were purchased from the Jackson Laboratory (stock no. 000651). Immunizations were prepared by mixing 10 μg of antigen and 100 μg of alum in 100 μL sterile tris-buffered saline (TBS, Sigma Aldrich, catalog no. T5912) per mouse unless otherwise specified. Antigen was loaded onto alum for 30 minutes on a tube rotator prior to immunization. When CpG 1826 or SMNP was added into the immunization, antigen was first loaded onto alum for 30 minutes on a rotator, after which 30 μg of CpG 1826 or 5 μg of SMNP was added into the immunization and incubated with antigen-alum formulations for 30 minutes prior to immunization. This dose of SMNP corresponds to 5 μg of Quil-A and 0.5 μg MPLA. Experiments in which antigen density was altered but the total alum dose remained the same, antigen was loaded onto alum at the indicated antigen:alum mass ratio for 30 minutes, and supplemented alum added just prior to immunization to bring the total alum dose to 200 μg per mouse. Mice were immunized subcutaneously at the tail base with 50 μL on each side of the tail base and were subsequently boosted 6 weeks post-prime.

### Antigen-Binding ELISA

Serum was collected from mice retro-orbitally using capillary tubes and stored at ˜20° C. until analysis. To determine serum IgG titers with RBD, Nunc Maxisorp plates (Invitrogen, catalog no. 44-2404-21) were coated with a rabbit anti-histag antibody (GenScript, catalog no. A00174-40) at 2 μg/ml for 4 hours at 25° C. in PBS and blocked with 2% BSA in PBS overnight at 4° C. Plates were washed with 0.05% Tween-20 PBS, and RBD was added at 2 μg/ml in 2% BSA in PBS for 2 hours. Serum dilutions (1:10 dilution followed by 1:50 dilution with 1:4 serial dilutions) were incubated in the plate for 2 hours. Plates are washed again, incubated with a goat anti-mouse IgG HRP-conjugated secondary (BioRad, catalog no. 1721011) at 1:5000 dilution, and then developed with 3,3′,5,5′-tetramethytlbenzidine (ThermoFisher, catalog no. 34028), stopped with 2N sulfuric acid, and immediately read (450 nm with 540 nm reference) on a BioTek Synergy2 plate reader. To determine serum IgG titers for mice immunized with RBDJ, protein was coated directly on Corning Costar High Binding 96-well plates (catalog no. 9018/3690) at 2 μg/ml in PBS overnight at 4° C. and blocked for 2 hours, and subsequently follow the protocol for RBD ELISAs. Isotype ELISAs followed the same protocol but used goat anti-mouse IgG1 HRP cross-adsorbed secondary antibody (Invitrogen, catalog no. A10551), goat anti-mouse IgG2a HRP cross-adsorbed secondary antibody (Invitrogen, catalog no. M32207), or goat anti-mouse IgG2b cross-adsorbed secondary antibody (Invitrogen, catalog no. M32407) at 1:2000 dilution.

### ACE2 Competition ELISA

Surrogate virus neutralization ELISAs (GenScript, catalog no. L00847A) were performed following the manufacturer's protocol. Briefly, mouse serum samples were diluted at 1:10 with 1:3 serial dilutions and mixed 1:1 with RBD-HRP for 30 minutes at 37° C. Samples were then added to hACE2 coated plates and incubated for 15 minutes at 37° C. Plates were developed for 15 minutes with 3,3′,5,5′-tetramethytlbenzidine, stopped with 1N sulfuric acid, and the absorbance at 450 nm was immediately read on a BioTek Synergy2 plate reader. ID₅₀ values were calculated using a nonlinear fit of individual dilution curves.

### Pseudovirus Neutralization Analysis

To assess neutralization in mouse serum samples, SARS-CoV-2 pseudoviruses expressing a luciferase reporter gene were generated similar to an approach described previously (59, 60). Briefly, HEK293T cells were co-transfected with the packaging plasmid psPAX2 (AIDS Resource and Reagent Program), luciferase reporter plasmid pLenti-CMV Puro-Luc (Addgene, catalog no. 17477), and spike protein expressing pcDNA3.1-SARS CoV-2 SΔCT using lipofectamine 2000 (ThermoFisher, catalog no. 11668030). Pseudotype viruses were collected from culture supernatants 48 hours post-transfection and purified by centrifugation and 0.45 μm filtration. To assess the neutralization activity of the mouse serum samples, serum was inactivated at 56° C. for 30 minutes. HEK293T-hACE2 cells were seeded overnight in 96-well tissue culture plates at a density of 1.75×10⁴ cells per well. Three-fold serial dilutions of heat inactivated serum samples were prepared and mixed with 50 μL of pseudovirus, followed by incubation at 37° C. for 1 hour before adding the mixture to HEK293T-hACE2 cells. After incubation for 48 hours, cells were lysed using Steady-Glo Luciferase Assay (Promega, catalog no. E2510) according to the manufacturer's instructions. SARS-CoV-2 pseudovirus neutralization titers were defined as the sample dilution at which a 50% reduction in relative light unit (RLU) was observed relative to the average virus control wells.

### ELISPOT Analysis

Bone marrow ELISPOTs were performed in mice 16 weeks post-prime following the manufacturer protocol (MabTech, catalog no. 3825-2A) unless otherwise specified. Briefly, 96-well PVDF ELISPOT plates (Millipore Sigma, catalog no. MSIPS4510) were treated with 35% ethanol prior to coating with anti-mouse IgG at 15 μg/ml in sterile PBS overnight at 4° C. Cells were isolated from the femur and tibia of mice, ACK lysed, and 70 μm filtered in complete media (RPMI 1640 containing 10% FBS, 100 U/ml penicillin-streptomycin, and 1 mM sodium pyruvate). The next day, plates were blocked with complete media for at least 30 minutes prior to adding cells with three technical replicates per mouse. For total IgG and antigen-specific IgG, 100,000 and 500,000 cells were added per well, respectively, and incubated at 37° C. with 5% CO₂ for 16 hours. Plates were then washed with PBS. Antigen-specific responses were determined by adding 1 μg/ml biotinylated RBD in PBS with 0.5% BSA to each well for 2 hours at 25° C. Total IgG responses were determined by adding 1 μg/ml anti-mouse IgG-biotin detection antibody in PBS with 0.5% BSA to each well for 2 hours at 25° C. Plates were washed again in PBS and incubated with 1:1000 streptavidin-alkaline phosphatase in PBS with 0.5% BSA for 1 hour at 25° C. After washing, plates were developed with BCIP/NBT substrate (MabTech, catalog no. 3650-10) and developed for 20 minutes, quenched with H₂O, and dried prior to quantification on an ImmunoSpot CTL Analyzer.

### Germinal Center and T Follicular Helper Responses

The inguinal lymph nodes were collected from immunized mice 14 days post-immunization unless otherwise specified. For germinal center analysis, cells were stained for viability (ThermoFisher Live/Dead Fixable Aqua, catalog no. L34957) and against CD3e (BV711, 145-2C11 clone; BioLegend, 100349), B220 (PE-Cy7, RA3-6B2 clone; BioLegend, catalog no. 103221), CD38 (FITC, 90 clone; BioLegend, catalog no. 102705), and GL7 (PerCP-Cy5.5, GL7 clone; BioLegend, catalog no. 144609), with antigen-specific staining completed using biotinylated RBD conjugated to streptavidin-BV421 (BioLegend, catalog no. 405226) and streptavidin-PE (BioLegend, catalog no. 405203). For T follicular helper analysis, cells were stained for viability (ThermoFisher LiveDead Fixable Aqua, catalog no. L34957) and against B220 (BV510, RA3-6B2 clone; BioLegend, catalog no. 103247), CD4 (FITC, GK1.5 clone; BioLegend, catalog no. 100405), CD44 (PE-Cy7, IM7 clone; BioLegend, catalog no. 103029), PD-1 (BV421, RMP1-30 clone; BD Biosciences, catalog no. 748268), and CXCR5 (PE, 2G8 clone; BD Biosciences, catalog no. 551960). Samples were analyzed by flow cytometry on a BD Celesta and analyzed on FlowJo.

### Cellular Uptake of Antigen

Mice were immunized with 10 μg of AlexaFluor™555 labeled antigen and 100 μg alum and 5 μg SMNP or 30 μg CpG, and the inguinal lymph nodes were collected 7 days post-immunization. Cells were stained for viability (ThermoFisher Live/Dead Fixable Near-IR, catalog no. L34975) and against CD3 (APC-Cy7, 17A2 clone; BioLegend, catalog no. 100221), NK1.1 (APC-Cy7, PK136 clone; BioLegend, catalog no. 108723), CD19 (PE-Cy7, 6D5 clone; BioLegend, catalog no. 115519), CD11b (BUV805, M1/70 clone; BD Biosciences, catalog no. 741934), CD11c (BUV496, HL3 clone; BD Biosciences, catalog no. 750483), Ly6C (BV650, HK1.4 clone; BioLegend, catalog no. 128049), Ly6G (BUV563, 1A8 clone; BD Biosciences, catalog no. 612921), F4/80 (BUV737, T45-2342 clone; BD Biosciences, catalog no. 749283), CD169 (BV421, 3D6.112 clone; BioLegend, catalog no. 142421), and MHC II (PE-Cy5, M5/114.15.2 clone; BioLegend, catalog no. 107611). Samples were analyzed by flow cytometry on a BD Symphony A3 and analyzed on FlowJo.

### Whole-Mouse Imaging of Vaccination Drainage

Mice were immunized subcutaneously at the tail base with fluorescently labeled antigen or adjuvant. Immunizations were prepared as described, using fluorescently labeled components as indicated. For studies including fluorescently labeled components, immunizations were prepared by loading antigen onto alum in sterile tris-buffered saline (TBS, Sigma Aldrich, catalog no. T5912) for 30 minutes on a tube rotator prior to adding co-adjuvants and incubating for 30 minutes on a tube rotator. Alum was labeled using 0.1 nmol of pSer₄-AlexaFluor™488 (SEQ ID NO: 120-AlexaFluor™488). Imaging was completed using a PerkinElmer Xenogen Spectrum in vivo imaging system (IVIS), and the fluorescent signal at the injection site was quantified using LivingImage software. The radiant efficiency was tracked longitudinally to monitor drainage from the injection site.

### Microscopy

Alum was incubated with AlexaFluor™555 labeled pSer₄-RBDJ (SEQ ID NO: 120-RBDJ) or pSer₄-AlexaFluor™488 (SEQ ID NO: 120-AlexaFluor™488) at 25° C. for 30 minutes in TBS. These solutions were mixed and incubated together for 2 days prior to imaging. Fluorescence images were acquired on an Applied Precision DeltaVision Microscope with a 100×/1.4 oil objective using the accompanying Softworx software. Image analysis was performed using Fiji (ImageJ version 2.1.0) by converting the images into a binary image, applying a Watershed transform, counting the number of particles (3D Objects Counter), and applying the Colocalization Threshold analysis to assess the number of particles for which there is colocalization of the two fluorescent signals. The number of alum particles with fluorescent colocalization was divided by the total number of alum particles detected in the image and reported as the fraction of particles with fluorescent colocalization.

### Statistical Analysis

All data were plotted and all statistical analyses were performed using GraphPad Prism 8 software (La Jolla, Calif.). All graphs display mean values, and the error bars represent the standard deviation unless otherwise specified. No samples or animals were excluded from the analyses. Statistical comparison was performed using a one-way ANOVA followed by Tukey's post-hoc test for single timepoint data and two-way ANOVA followed by Tukey's post-hoc test for multi-timepoint longitudinal data. Statistical analysis of antibody titer was completed using log-transformed data. Data were considered statistically significant if the p-value was less than 0.05.

### REFERENCES

1. X. Ou, Y. Liu, X. Lei, P. Li, D. Mi, L. Ren, L. Guo, R. Guo, T. Chen, J. Hu, Z. Xiang, Z. Mu, X. Chen, J. Chen, K. Hu, Q. Jin, J. Wang, Z. Qian, Characterization of spike glycoprotein of SARS-CoV-2 on virus entry and its immune cross-reactivity with SARS-CoV. Nat Commun. 11, 1620 (2020).
2. W. Tai, L. He, X. Zhang, J. Pu, D. Voronin, S. Jiang, Y. Zhou, L. Du, Characterization of the receptor-binding domain (RBD) of 2019 novel coronavirus: implication for development of RBD protein as a viral attachment inhibitor and vaccine. Cell Mol Immunol. 17, 613-620 (2020).
3. K. McMahan, J. Yu, N. B. Mercado, C. Loos, L. H. Tostanoski, A. Chandrashekar, J. Liu, L. Peter, C. Atyeo, A. Zhu, E. A. Bondzie, G. Dagotto, M. S. Gebre, C. Jacob-Dolan, Z. Li, F. Nampanya, S. Patel, L. Pessaint, A. V. Ry, K. Blade, J. Yalley-Ogunro, M. Cabus, R. Brown, A. Cook, E. Teow, H. Andersen, M. G. Lewis, D. A. Lauffenburger, G. Alter, D. H. Barouch, Correlates of protection against SARS-CoV-2 in rhesus macaques. Nature. 590, 630-634 (2021).
4. T. F. Rogers, F. Zhao, D. Huang, N. Beutler, A. Burns, W. He, O. Limbo, C. Smith, G. Song, J. Woehl, L. Yang, R. K. Abbott, S. Callaghan, E. Garcia, J. Hurtado, M. Parren, L. Peng, S. Ramirez, J. Ricketts, M. J. Ricciardi, S. A. Rawlings, N. C. Wu, M. Yuan, D. M. Smith, D. Nemazee, J. R. Teijaro, J. E. Voss, I. A. Wilson, R. Andrabi, B. Briney, E. Landais, D. Sok, J. G. Jardine, D. R. Burton, Isolation of potent SARS-CoV-2 neutralizing antibodies and protection from disease in a small animal model. Science. 369, 956-963 (2020).
5. S. J. Zost, P. Gilchuk, J. B. Case, E. Binshtein, R. E. Chen, J. P. Nkolola, A. Schäfer, J. X. Reidy, A. Trivette, R. S. Nargi, R. E. Sutton, N. Suryadevara, D. R. Martinez, L. E. Williamson, E. C. Chen, T. Jones, S. Day, L. Myers, A. O. Hassan, N. M. Kafai, E. S. Winkler, J. M. Fox, S. Shrihari, B. K. Mueller, J. Meiler, A. Chandrashekar, N. B. Mercado, J. J. Steinhardt, K. Ren, Y.-M. Loo, N. L. Kallewaard, B. T. McCune, S. P. Keeler, M. J. Holtzman, D. H. Barouch, L. E. Gralinski, R. S. Baric, L. B. Thackray, M. S. Diamond, R. H. Carnahan, J. E. Crowe, Potently neutralizing and protective human antibodies against SARS-CoV-2. Nature. 584, 443-449 (2020).
6. A. Addetia, K. H. D. Crawford, A. Dingens, H. Zhu, P. Roychoudhury, M.-L. Huang, K. R. Jerome, J. D. Bloom, A. L. Greninger, Neutralizing Antibodies Correlate with Protection from SARS-CoV-2 in Humans during a Fishery Vessel Outbreak with a High Attack Rate. J Clin Microbiol. 58 (2020), doi:10.1128/jcm.02107-20.
7. P. Jin, J. Li, H. Pan, Y. Wu, F. Zhu, Immunological surrogate endpoints of COVID-2019 vaccines: the evidence we have versus the evidence we need. Signal Transduct Target Ther. 6, 48 (2021).
8. N. B. Mercado, R. Zahn, F. Wegmann, C. Loos, A. Chandrashekar, J. Yu, J. Liu, L. Peter, K. McMahan, L. H. Tostanoski, X. He, D. R. Martinez, L. Rutten, R. Bos, D. van Manen, J. Vellinga, J. Custers, J. P. Langedijk, T. Kwaks, M. J. G. Bakkers, D. Zuijdgeest, S. K. R. Huber, C. Atyeo, S. Fischinger, J. S. Burke, J. Feldman, B. M. Hauser, T. M. Caradonna, E. A. Bondzie, G. Dagotto, M. S. Gebre, E. Hoffman, C. Jacob-Dolan, M. Kirilova, Z. Li, Z. Lin, S. H. Mahrokhian, L. F. Maxfield, F. Nampanya, R. Nityanandam, J. P. Nkolola, S. Patel, J. D. Ventura, K. Verrington, H. Wan, L. Pessaint, A. V. Ry, K. Blade, A. Strasbaugh, M. Cabus, R. Brown, A. Cook, S. Zouantchangadou, E. Teow, H. Andersen, M. G. Lewis, Y. Cai, B. Chen, A. G. Schmidt, R. K. Reeves, R. S. Baric, D. A. Lauffenburger, G. Alter, P. Stoffels, M. Mammen, J. V. Hoof, H. Schuitemaker, D. H. Barouch, Single-shot Ad26 vaccine protects against SARS-CoV-2 in rhesus macaques. Nature. 586, 583-588 (2020).
9. F. Krammer, SARS-CoV-2 vaccines in development. Nature. 586, 516-527 (2020).
10. J. Yang, W. Wang, Z. Chen, S. Lu, F. Yang, Z. Bi, L. Bao, F. Mo, X. Li, Y. Huang, W. Hong, Y. Yang, Y. Zhao, F. Ye, S. Lin, W. Deng, H. Chen, H. Lei, Z. Zhang, M. Luo, H. Gao, Y. Zheng, Y. Gong, X. Jiang, Y. Xu, Q. Lv, D. Li, M. Wang, F. Li, S. Wang, G. Wang, P. Yu, Y. Qu, L. Yang, H. Deng, A. Tong, J. Li, Z. Wang, J. Yang, G. Shen, Z. Zhao, Y. Li, J. Luo, H. Liu, W. Yu, M. Yang, J. Xu, J. Wang, H. Li, H. Wang, D. Kuang, P. Lin, Z. Hu, W. Guo, W. Cheng, Y. He, X. Song, C. Chen, Z. Xue, S. Yao, L. Chen, X. Ma, S. Chen, M. Gou, W. Huang, Y. Wang, C. Fan, Z. Tian, M. Shi, F.-S. Wang, L. Dai, M. Wu, G. Li, G. Wang, Y. Peng, Z. Qian, C. Huang, J. Y.-N. Lau, Z. Yang, Y. Wei, X. Cen, X. Peng, C. Qin, K. Zhang, G. Lu, X. Wei, A vaccine targeting the RBD of the S protein of SARS-CoV-2 induces protective immunity. Nature. 586, 572-577 (2020).
11. L. Dai, T. Zheng, K. Xu, Y. Han, L. Xu, E. Huang, Y. An, Y. Cheng, S. Li, M. Liu, M. Yang, Y. Li, H. Cheng, Y. Yuan, W. Zhang, C. Ke, G. Wong, J. Qi, C. Qin, J. Yan, G. F. Gao, A Universal Design of Betacoronavirus Vaccines against COVID-19, MERS, and SARS. Cell. 182, 722-733.e11 (2020).
12. D. F. Robbiani, C. Gaebler, F. Muecksch, J. C. C. Lorenzi, Z. Wang, A. Cho, M. Agudelo, C. O. Barnes, A. Gazumyan, S. Finkin, T. Hagglof, T. Y. Oliveira, C. Viant, A. Hurley, H.-H. Hoffmann, K. G. Millard, R. G. Kost, M. Cipolla, K. Gordon, F. Bianchini, S. T. Chen, V. Ramos, R. Patel, J. Dizon, I. Shimeliovich, P. Mendoza, H. Hartweger, L. Nogueira, M. Pack, J. Horowitz, F. Schmidt, Y. Weisblum, E. Michailidis, A. W. Ashbrook, E. Waltari, J. E. Pak, K. E. Huey-Tubman, N. Koranda, P. R. Hoffman, A. P. West, C. M. Rice, T. Hatziioannou, P. J. Bjorkman, P. D. Bieniasz, M. Caskey, M. C. Nussenzweig, Convergent antibody responses to SARS-CoV-2 in convalescent individuals. Nature. 584, 437-442 (2020).
13. H. Tegally, E. Wilkinson, M. Giovanetti, A. Iranzadeh, V. Fonseca, J. Giandhari, D. Doolabh, S. Pillay, E. J. San, N. Msomi, K. Mlisana, A. von Gottberg, S. Walaza, M. Allam, A. Ismail, T. Mohale, A. J. Glass, S. Engelbrecht, G. V. Zyl, W. Preiser, F. Petruccione, A. Sigal, D. Hardie, G. Marais, M. Hsiao, S. Korsman, M.-A. Davies, L. Tyers, I. Mudau, D. York, C. Maslo, D. Goedhals, S. Abrahams, O. Laguda-Akingba, A. Alisoltani-Dehkordi, A. Godzik, C. K. Wibmer, B. T. Sewell, J. Lourengo, L. C. J. Alcantara, S. L. K. Pond, S. Weaver, D. Martin, R. J. Lessells, J. N. Bhiman, C. Williamson, T. de Oliveira, Emergence and rapid spread of a new severe acute respiratory syndrome-related coronavirus 2 (SARS-CoV-2) lineage with multiple spike mutations in South Africa. medRxiv (2020), doi:10.1101/2020.12.21.20248640.
14. T. Alpert, A. F. Brito, E. Lasek-Nesselquist, J. Rothman, A. L. Valesano, M. J. MacKay, M. E. Petrone, M. I. Breban, A. E. Watkins, C. B. F. Vogels, C. C. Kalinich, S. Dellicour, A. Russell, J. P. Kelly, M. Shudt, J. Plitnick, E. Schneider, W. J. Fitzsimmons, G. Khullar, J. Metti, J. T. Dudley, M. Nash, N. Beaubier, J. Wang, C. Liu, P. Hui, A. Muyombwe, R. Downing, J. Razeq, S. M. Bart, A. Grills, S. M. Morrison, S. Murphy, C. Neal, E. Laszlo, H. Rennert, M. Cushing, L. Westblade, P. Velu, A. Craney, L. Cong, D. R. Peaper, M. L. Landry, P. W. Cook, J. R. Fauver, C. E. Mason, A. S. Lauring, K. St. George, D. R. MacCannell, N. D. Grubaugh, Early introductions and transmission of SARS-CoV-2 variant B.1.1.7 in the United States. Cell (2021), doi:10.1016/j.cell.2021.03.061.
15. M. Krutikov, A. Hayward, L. Shallcross, Spread of a Variant SARS-CoV-2 in Long-Term Care Facilities in England. New Engl J Med (2021), doi:10.1056/nejmc2035906.
16. A. Rambaut, N. Loman, O. Pybus, W. Barclay, J. Barrett, A. Carabelli, T. Connor, T. Peacock, D. L. Robertson, E. Volz, C.-19 G. C. U. (CoG-UK), Preliminary genomic characterisation of an emergent SARS-CoV-2 lineage in the UK defined by a novel set of spike mutations. (2021), (available at https://virological.org/t/preliminary-genomic-characterisation-of-an-emergent-sars-cov-2-lineage-in-the-uk-defined-by-a-novel-set-of-spike-mutations/563).
17. D. Planas, T. Bruel, L. Grzelak, F. Guivel-Benhassine, I. Staropoli, F. Porrot, C. Planchais, J. Buchrieser, M. M. Rajah, E. Bishop, M. Albert, F. Donati, M. Prot, S. Behillil, V. Enouf, M. Maquart, M. Smati-Lafarge, E. Varon, F. Schortgen, L. Yahyaoui, M. Gonzalez, J. D. Sëze, H. Péré, D. Veyer, A. Seve, E. Simon-Lorière, S. Fafi-Kremer, K. Stefic, H. Mouquet, L. Hocqueloux, S. van der Werf, T. Prazuck, O. Schwartz, Sensitivity of infectious SARS-CoV-2 B.1.1.7 and B.1.351 variants to neutralizing antibodies. Nat Med, 1-8 (2021).
18. A. Muik, A.-K. Wallisch, B. Sanger, K. A. Swanson, J. M0hl, W. Chen, H. Cai, D. Maurus, R. Sarkar, Ö. Tireci, P. R. Dormitzer, U. Şahin, Neutralization of SARS-CoV-2 lineage B.1.1.7 pseudovirus by BNT162b2 vaccine-elicited human sera. Science. 371, 1152-1153 (2021).
19. V. V. Edara, K. Floyd, L. Lai, M. Gardner, W. Hudson, A. Piantadosi, J. J. Waggoner, A. Babiker, R. Ahmed, X. Xie, K. Lokugamage, V. Menachery, P.-Y. Shi, M. S. Suthar, Infection and mRNA-1273 vaccine antibodies neutralize SARS-CoV-2 UK variant. medRxiv (2021), doi:10.1101/2021.02.02.21250799.
20. R. E. Chen, X. Zhang, J. B. Case, E. S. Winkler, Y. Liu, L. A. VanBlargan, J. Liu, J. M. Errico, X. Xie, N. Suryadevara, P. Gilchuk, S. J. Zost, S. Tahan, L. Droit, J. S. Turner, W. Kim, A. J. Schmitz, M. Thapa, D. Wang, A. C. M. Boon, R. M. Presti, J. A. O'Halloran, A. H. J. Kim, P. Deepak, D. Pinto, D. H. Fremont, J. E. Crowe, D. Corti, H. W. Virgin, A. H. Ellebedy, P.-Y. Shi, M. S. Diamond, Resistance of SARS-CoV-2 variants to neutralization by monoclonal and serum-derived polyclonal antibodies. Na tMed. 27, 717-726 (2021).
21. D. Weissman, M.-G. Alameh, T. de Silva, P. Collini, H. Hornsby, R. Brown, C. C. LaBranche, R. J. Edwards, L. Sutherland, S. Santra, K. Mansouri, S. Gobeil, C. McDanal, N. Pardi, N. Hengartner, P. J. C. Lin, Y. Tam, P. A. Shaw, M. G. Lewis, C. Boesler, U. Şahin, P. Acharya, B. F. Haynes, B. Korber, D. C. Montefiori, D614G Spike Mutation Increases SARS CoV-2 Susceptibility to Neutralization. Cell Host Microbe. 29, 23-31.e4 (2020).
22. F. Lu, I. Boutselis, R. F. Borch, H. HogenEsch, Control of antigen-binding to aluminum adjuvants and the immune response with a novel phosphonate linker. Vaccine. 31, 4362-4367 (2013).
23. S. L. Hem, H. HogenEsch, Relationship between physical and chemical properties of aluminum-containing adjuvants and immunopotentiation. Expert Rev Vaccines. 6, 685-698 (2014).
24. B. Hansen, A. Sokolovska, H. HogenEsch, S. L. Hem, Relationship between the strength of antigen adsorption to an aluminum-containing adjuvant and the immune response. Vaccine. 25, 6618-6624 (2007).
25. S. Iyer, H. HogenEsch, S. L. Hem, Relationship between the degree of antigen adsorption to aluminum hydroxide adjuvant in interstitial fluid and antibody production. Vaccine. 21, 1219-1223 (2003).
26. T. J. Moyer, Y. Kato, W. Abraham, J. Y. H. Chang, D. W. Kulp, N. Watson, H. L. Turner, S. Menis, R. K. Abbott, J. N. Bhiman, M. B. Melo, H. A. Simon, S. H.-D. la Mata, S. Liang, G. Seumois, Y. Agarwal, N. Li, D. R. Burton, A. B. Ward, W. R. Schief, S. Crotty, D. J. Irvine, Engineered immunogen binding to alum adjuvant enhances humoral immunity. Nat Med. 26, 430-440 (2020).
27. M. Silva, Y. Kato, M. B. Melo, I. Phung, B. L. Freeman, Z. Li, K. Roh, J. V. Wijnbergen, H. Watkins, C. A. Enemuo, B. Hartwell, J. Chang, S. Xiao, K. A. Rodrigues, K. M. Cirelli, N. Li, S. Haupt, A. Aung, B. Cossette, S. Kataria, R. Bastidas, J. Bhiman, C. Linde, A. Thomas, J. Bals, D. G. Carnathan, D. Lingwood, D. Burton, D. Alter, T. Padera, A. Belcher, W. R. Schief, G. Silvestri, R. Ruprecht, S. Crotty, D. J. Irvine, A potent saponin and TLR agonist particulate vaccine adjuvant alters lymphatic flow and lymph node antigen accumulation. Science Immunology (in review) (2021).
28. M. Yuan, N. C. Wu, X. Zhu, C.-C. D. Lee, R. T. Y. So, H. Lv, C. K. P. Mok, I. A. Wilson, A highly conserved cryptic epitope in the receptor binding domains of SARS-CoV-2 and SARS-CoV. Science. 368, 630-633 (2020).
29. N. C. Dalvie, S. A. Rodriguez-Aponte, B. L. Hartwell, L. H. Tostanoski, A. M. Biedermann, L. E. Crowell, K. Kaur, O. Kumru, L. Carter, J. Yu, A. Chang, K. McMahan, T. Courant, C. Lebas, A. A. Lemnios, K. A. Rodrigues, M. Silva, R. S. Johnston, C. A. Naranjo, M. K. Tracey, J. R. Brady, C. A. Whittaker, D. Yun, S. Kar, M. Porto, M. Lok, H. Andersen, M. G. Lewis, K. R. Love, D. L. Camp, J. M. Silverman, H. Kleanthous, S. B. Joshi, D. B. Volkin, P. M. Dubois, N. Collin, N. P. King, D. H. Barouch, D. J. Irvine, J. C. Love, Engineered SARS-CoV-2 receptor binding domain improves immunogenicity in mice and elicits protective immunity in hamsters. bioRxiv (2021), doi:10.1101/2021.03.03.433558.
30. C. T. Johnston, S. Wang, S. L. Hem, Measuring the surface area of aluminum hydroxide adjuvant. J Pharm Sci. 91, 1702-1706 (2002).
31. J. D. Campbell, Y. Cho, M. L. Foster, H. Kanzler, M. A. Kachura, J. A. Lum, M. J. Ratcliffe, A. Sathe, A. J. Leishman, A. Bahl, M. McHale, R. L. Coffman, E. M. Hessel, CpG-containing immunostimulatory DNA sequences elicit TNF-α-dependent toxicity in rodents but not in humans. J Clin Invest. 119, 2564-2576 (2009).
32. J. Mateus, A. Grifoni, A. Tarke, J. Sidney, S. I. Ramirez, J. M. Dan, Z. C. Burger, S. A. Rawlings, D. M. Smith, E. Phillips, S. Mallal, M. Lammers, P. Rubiro, L. Quiambao, A. Sutherland, E. D. Yu, R. da S. Antunes, J. Greenbaum, A. Frazier, A. J. Markmann, L. Premkumar, A. de Silva, B. Peters, S. Crotty, A. Sette, D. Weiskopf, Selective and cross-reactive SARS-CoV-2 T cell epitopes in unexposed humans. Science. 370, 89-94 (2020).
33. J. Alexander, J. Sidney, S. Southwood, J. Ruppert, C. Oseroff, A. Maewal, K. Snoke, H. M. Serra, R. T. Kubo, A. Sette, H. M. Grey, Development of high potency universal DR-restricted helper epitopes by modification of high affinity DR-blocking peptides. Immunity. 1, 751-761 (1994).
34. J. Alexander, M.-F. del Guercio, A. Maewal, L. Qiao, J. Fikes, R. W. Chesnut, J. Paulson, D. R. Bundle, S. DeFrees, A. Sette, Linear PADRE T Helper Epitope and Carbohydrate B Cell Epitope Conjugates Induce Specific High Titer IgG Antibody Responses. J Immunol. 164, 1625-1633 (2000).
35. E. D. Franke, S. L. Hoffman, J. B. Sacci, R. Wang, Y. Charoenvit, E. Appella, R. Chesnut, J. Alexander, M.-F. D. Guercio, A. Sette, Pan D R binding sequence provides T-cell help for induction of protective antibodies against Plasmodium yoelii sporozoites. Vaccine. 17, 1201-1205 (1999).
36. K. M. Cirelli, D. G. Carnathan, B. Nogal, J. T. Martin, O. L. Rodriguez, A. A. Upadhyay, C. A. Enemuo, E. H. Gebru, Y. Choe, F. Viviano, C. Nakao, M. G. Pauthner, S. Reiss, C. A. Cottrell, M. L. Smith, R. Bastidas, W. Gibson, A. N. Wolabaugh, M. B. Melo, B. Cossette, V. Kumar, N. B. Patel, T. Tokatlian, S. Menis, D. W. Kulp, D. R. Burton, B. Murrell, W. R. Schief, S. E. Bosinger, A. B. Ward, C. T. Watson, G. Silvestri, D. J. Irvine, S. Crotty, Slow Delivery Immunization Enhances HIV Neutralizing Antibody and Germinal Center Responses via Modulation of Immunodominance. Cell. 180, 206 (2019).
37. M. Pauthner, C. Havenar-Daughton, D. Sok, J. P. Nkolola, R. Bastidas, A. V. Boopathy, D. G. Carnathan, A. Chandrashekar, K. M. Cirelli, C. A. Cottrell, A. M. Eroshkin, J. Guenaga, K. Kaushik, D. W. Kulp, J. Liu, L. E. McCoy, A. L. Oom, G. Ozorowski, K. W. Post, S. K. Sharma, J. M. Steichen, S. W. de Taeye, T. Tokatlian, A. T. de la Peña, S. T. Butera, C. C. LaBranche, D. C. Montefiori, G. Silvestri, I. A. Wilson, D. J. Irvine, R. W. Sanders, W. R. Schief, A. B. Ward, R. T. Wyatt, D. H. Barouch, S. Crotty, D. R. Burton, Elicitation of Robust Tier 2 Neutralizing Antibody Responses in Nonhuman Primates by HIV Envelope Trimer Immunization Using Optimized Approaches. Immunity. 46, 1073-1088.e6 (2017).
38. H. H. Tam, M. B. Melo, M. Kang, J. M. Pelet, V. M. Ruda, M. H. Foley, J. K. Hu, S. Kumari, J. Crampton, A. D. Baldeon, R. W. Sanders, J. P. Moore, S. Crotty, R. Langer, D. G. Anderson, A. K. Chakraborty, D. J. Irvine, Sustained antigen availability during germinal center initiation enhances antibody responses to vaccination. Proc National Acad Sci. 113, E6639-E6648 (2016).
39. P. Johansen, T. Storni, L. Rettig, Z. Qiu, A. Der-Sarkissian, K. A. Smith, V. Manolova, K. S. Lang, G. Senti, B. Müllhaupt, T. Gerlach, R. F. Speck, A. Bot, T. M. Kündig, Antigen kinetics determines immune reactivity. Proc National Acad Sci. 105, 5189-5194 (2008).
40. H. Jiang, Q. Wang, L. Li, Q. Zeng, H. Li, T. Gong, Z. Zhang, X. Sun, Turning the Old Adjuvant from Gel to Nanoparticles to Amplify CD8+ T Cell Responses. Adv Sci. 5, 1700426 (2018).
41. C. L. B. Millan, R. Weeratna, A. M. Krieg, C.-A. Siegrist, H. L. Davis, CpG DNA can induce strong Th1 humoral and cell-mediated immune responses against hepatitis B surface antigen in young mice. Proc National Acad Sci. 95, 15553-15558 (1998).
42. T.-Y. Kuo, M.-Y. Lin, R. L. Coffman, J. D. Campbell, P. Traquina, Y.-J. Lin, L. T.-C. Liu, J. Cheng, Y.-C. Wu, C.-C. Wu, W.-H. Tang, C.-G. Huang, K.-C. Tsao, C. Chen, Development of CpG-adjuvanted stable prefusion SARS-CoV-2 spike antigen as a subunit vaccine against COVID-19. Sci Rep-uk. 10, 20085 (2020).
43. P. S. Arunachalam, A. C. Walls, N. Golden, C. Atyeo, S. Fischinger, C. Li, P. Aye, M. J. Navarro, L. Lai, V. V. Edara, K. Roltgen, K. Rogers, L. Shirreff, D. E. Ferrell, S. Wrenn, D. Pettie, J. C. Kraft, M. C. Miranda, E. Kepl, C. Sydeman, N. Brunette, M. Murphy, B. Fiala, L. Carter, A. G. White, M. Trisal, C.-L. Hsieh, K. Russell-Lodrigue, C. Monjure, J. Dufour, S. Spencer, L. Doyle-Meyer, R. P. Bohm, N. J. Maness, C. Roy, J. A. Plante, K. S. Plante, A. Zhu, M. J. Gorman, S. Shin, X. Shen, J. Fontenot, S. Gupta, D. T. O'Hagan, R. V. D. Most, R. Rappuoli, R. L. Coffman, D. Novack, J. S. McLellan, S. Subramaniam, D. Montefiori, S. D. Boyd, J. L. Flynn, G. Alter, F. Villinger, H. Kleanthous, J. Rappaport, M. S. Suthar, N. P. King, D. Veesler, B. Pulendran, Adjuvanting a subunit COVID-19 vaccine to induce protective immunity. Nature, 1-10 (2021).
44. P. Richmond, L. Hatchuel, M. Dong, B. Ma, B. Hu, I. Smolenov, P. Li, P. Liang, H. H. Han, J. Liang, R. Clemens, Safety and immunogenicity of S-Trimer (SCB-2019), a protein subunit vaccine candidate for COVID-19 in healthy adults: a phase 1, randomised, double-blind, placebo-controlled trial. Lancet. 397, 682-694 (2021).
45. X. Ma, F. Zou, F. Yu, R. Li, Y. Yuan, Y. Zhang, X. Zhang, J. Deng, T. Chen, Z. Song, Y. Qiao, Y. Zhan, J. Liu, J. Zhang, X. Zhang, Z. Peng, Y. Li, Y. Lin, L. Liang, G. Wang, Y. Chen, Q. Chen, T. Pan, X. He, H. Zhang, Nanoparticle Vaccines Based on the Receptor Binding Domain (RBD) and Heptad Repeat (HR) of SARS-CoV-2 Elicit Robust Protective Immune Responses. Immunity (2020), doi:10.1016/j.immuni.2020.11.015.
46. M. Mandolesi, D. J. Sheward, L. Hanke, J. Ma, P. Pushparaj, L. P. Vidakovics, C. Kim, M. Adori, K. Lenart, K. Lore, X. C. Dopico, J. M. Coquet, G. M. McInerney, G. B. K. Hedestam, B. Murrell, SARS-CoV-2 protein subunit vaccination of mice and rhesus macaques elicits potent and durable neutralizing antibody responses. Cell Reports Medicine, 100252 (2021).
47. P. J. M. Brouwer, M. Brinkkemper, P. Maisonnasse, N. Dereuddre-Bosquet, M. Grobben, M. Claireaux, M. de Gast, R. Marlin, V. Chesnais, S. Diry, J. D. Allen, Y. Watanabe, J. M. Giezen, G. Kerster, H. L. Turner, K. van der Straten, C. A. van der Linden, Y. Aldon, T. Naninck, I. Bontjer, J. A. Burger, M. Poniman, A. Z. Mykytyn, N. M. A. Okba, E. E. Schermer, M. J. van Breemen, R. Ravichandran, T. G. Caniels, J. van Schooten, N. Kahlaoui, V. Contreras, J. Lemaitre, C. Chapon, R. H. T. Fang, J. Villaudy, K. Sliepen, Y. U. van der Velden, B. L. Haagmans, G. J. de Bree, E. Ginoux, A. B. Ward, M. Crispin, N. P. King, S. van der Werf, M. J. van Gils, R. L. Grand, R. W. Sanders, Two-component spike nanoparticle vaccine protects macaques from SARS-CoV-2 infection. Cell (2021), doi:10.1016/j.cell.2021.01.035.
48. A. B. Vogel, I. Kanevsky, Y. Che, K. A. Swanson, A. Muik, M. Vormehr, L. M. Kranz, K. C. Walzer, S. Hein, A. Guler, J. Loschko, M. S. Maddur, A. Ota-Setlik, K. Tompkins, J. Cole, B. G. Lui, T. Ziegenhals, A. Plaschke, D. Eisel, S. C. Dany, S. Fesser, S. Erbar, F. Bates, D. Schneider, B. Jesionek, B. Sanger, A.-K. Wallisch, Y. Feuchter, H. Junginger, S. A. Krumm, A. P. Heinen, P. Adams-Quack, J. Schlereth, S. Schille, C. Kröner, R. de la C. G. Garcia, T. Hiller, L. Fischer, R. S. Sellers, S. Choudhary, O. Gonzalez, F. Vascotto, M. R. Gutman, J. A. Fontenot, S. Hall-Ursone, K. Brasky, M. C. Griffor, S. Han, A. A. H. Su, J. A. Lees, N. L. Nedoma, E. H. Mashalidis, P. V. Sahasrabudhe, C. Y. Tan, D. Pavliakova, G. Singh, C. Fontes-Garfias, M. Pride, I. L. Scully, T. Ciolino, J. Obregon, M. Gazi, R. Carrion, K. J. Alfson, W. V. Kalina, D. Kaushal, P.-Y. Shi, T. Klamp, C. Rosenbaum, A. N. Kuhn, Ö. Tureci, P. R. Dormitzer, K. U. Jansen, U. Sahin, BNT162b vaccines protect rhesus macaques from SARS-CoV-2. Nature. 592, 283-289 (2021).
49. K. S. Corbett, B. Flynn, K. E. Foulds, J. R. Francica, S. Boyoglu-Barnum, A. P. Werner, B. Flach, S. O'Connell, K. W. Bock, M. Minai, B. M. Nagata, H. Anderson, D. R. Martinez, A. T. Noe, N. Douek, M. M. Donaldson, N. N. Nji, G. S. Alvarado, D. K. Edwards, D. R. Flebbe, E. Lamb, N. A. Doria-Rose, B. C. Lin, M. K. Louder, S. O'Dell, S. D. Schmidt, E. Phung, L. A. Chang, C. Yap, J.-P. M. Todd, L. Pessaint, A. V. Ry, S. Browne, J. Greenhouse, T. Putman-Taylor, A. Strasbaugh, T.-A. Campbell, A. Cook, A. Dodson, K. Steingrebe, W. Shi, Y. Zhang, O. M. Abiona, L. Wang, A. Pegu, E. S. Yang, K. Leung, T. Zhou, I.-T. Teng, A. Widge, I. Gordon, L. Novik, R. A. Gillespie, R. J. Loomis, J. I. Moliva, G. Stewart-Jones, S. Himansu, W.-P. Kong, M. C. Nason, K. M. Morabito, T. J. Ruckwardt, J. E. Ledgerwood, M. R. Gaudinski, P. D. Kwong, J. R. Mascola, A. Carfi, M. G. Lewis, R. S. Baric, A. McDermott, I. N. Moore, N. J. Sullivan, M. Roederer, R. A. Seder, B. S. Graham, Evaluation of the mRNA-1273 Vaccine against SARS-CoV-2 in Nonhuman Primates. New Engl J Med (2020), doi:10.1056/nejmoa2024671.
50. N. B. Mercado, R. Zahn, F. Wegmann, C. Loos, A. Chandrashekar, J. Yu, J. Liu, L. Peter, K. McMahan, L. H. Tostanoski, X. He, D. R. Martinez, L. Rutten, R. Bos, D. van Manen, J. Vellinga, J. Custers, J. P. Langedijk, T. Kwaks, M. J. G. Bakkers, D. Zuijdgeest, S. K. R. Huber, C. Atyeo, S. Fischinger, J. S. Burke, J. Feldman, B. M. Hauser, T. M. Caradonna, E. A. Bondzie, G. Dagotto, M. S. Gebre, E. Hoffman, C. Jacob-Dolan, M. Kirilova, Z. Li, Z. Lin, S. H. Mahrokhian, L. F. Maxfield, F. Nampanya, R. Nityanandam, J. P. Nkolola, S. Patel, J. D. Ventura, K. Verrington, H. Wan, L. Pessaint, A. V. Ry, K. Blade, A. Strasbaugh, M. Cabus, R. Brown, A. Cook, S. Zouantchangadou, E. Teow, H. Andersen, M. G. Lewis, Y. Cai, B. Chen, A. G. Schmidt, R. K. Reeves, R. S. Baric, D. A. Lauffenburger, G. Alter, P. Stoffels, M. Mammen, J. V. Hoof, H. Schuitemaker, D. H. Barouch, Single-shot Ad26 vaccine protects against SARS-CoV-2 in rhesus macaques. Nature, 1-11 (2020).
51. J. R. Francica, B. J. Flynn, K. E. Foulds, A. T. Noe, A. P. Werner, I. N. Moore, M. Gagne, T. S. Johnston, C. Tucker, R. L. Davis, B. Flach, S. O'Connell, S. F. Andrew, E. Lamb, D. R. Flebbe, S. T. Nurmukhambetova, M. M. Donaldson, J.-P. M. Todd, A. L. Zhu, C. Atyeo, S. Fischinger, M. J. Gorman, S. Shin, V. V. Edara, K. Floyd, L. Lai, A. Tylor, E. McCarthy, V. Lecouturier, S. Ruiz, C. Berry, T. Tibbitts, H. Andersen, A. Cook, A. Dodson, L. Pessaint, A. V. Ry, M. Koutsoukos, C. Gutzeit, I.-T. Teng, T. Zhou, D. Li, B. F. Haynes, P. D. Kwong, A. McDermott, M. G. Lewis, T. M. Fu, R. Chicz, R. van der Most, K. S. Corbett, M. S. Suthar, G. Alter, M. Roederer, N. J. Sullivan, D. C. Douek, B. S. Graham, D. Casimiro, R. A. Seder, Vaccination with SARS-CoV-2 Spike Protein and AS03 Adjuvant Induces Rapid Anamnestic Antibodies in the Lung and Protects Against Virus Challenge in Nonhuman Primates. bioRxiv (2021), doi:10.1101/2021.03.02.433390.
52. M. Guebre-Xabier, N. Patel, J.-H. Tian, B. Zhou, S. Maciejewski, K. Lam, A. D. Portnoff, M. J. Massare, M. B. Frieman, P. A. Piedra, L. Ellingsworth, G. Glenn, G. Smith, NVX-CoV2373 vaccine protects cynomolgus macaque upper and lower airways against SARS-CoV-2 challenge. Vaccine. 38, 7892-7896 (2020).
53. C. Keech, G. Albert, I. Cho, A. Robertson, P. Reed, S. Neal, J. S. Plested, M. Zhu, S. Cloney-Clark, H. Zhou, G. Smith, N. Patel, M. B. Frieman, R. E. Haupt, J. Logue, M. McGrath, S. Weston, P. A. Piedra, C. Desai, K. Callahan, M. Lewis, P. Price-Abbott, N. Formica, V. Shinde, L. Fries, J. D. Lickliter, P. Griffin, B. Wilkinson, G. M. Glenn, Phase 1-2 Trial of a SARS-CoV-2 Recombinant Spike Protein Nanoparticle Vaccine. New Engl J Med. 383, 2320-2332 (2020).
54. J. Yang, W. Wang, Z. Chen, S. Lu, F. Yang, Z. Bi, L. Bao, F. Mo, X. Li, Y. Huang, W. Hong, Y. Yang, Y. Zhao, F. Ye, S. Lin, W. Deng, H. Chen, H. Lei, Z. Zhang, M. Luo, H. Gao, Y. Zheng, Y. Gong, X. Jiang, Y. Xu, Q. Lv, D. Li, M. Wang, F. Li, S. Wang, G. Wang, P. Yu, Y. Qu, L. Yang, H. Deng, A. Tong, J. Li, Z. Wang, J. Yang, G. Shen, Z. Zhao, Y. Li, J. Luo, H. Liu, W. Yu, M. Yang, J. Xu, J. Wang, H. Li, H. Wang, D. Kuang, P. Lin, Z. Hu, W. Guo, W. Cheng, Y. He, X. Song, C. Chen, Z. Xue, S. Yao, L. Chen, X. Ma, S. Chen, M. Gou, W. Huang, Y. Wang, C. Fan, Z. Tian, M. Shi, F.-S. Wang, L. Dai, M. Wu, G. Li, G. Wang, Y. Peng, Z. Qian, C. Huang, J. Y.-N. Lau, Z. Yang, Y. Wei, X. Cen, X. Peng, C. Qin, K. Zhang, G. Lu, X. Wei, A vaccine targeting the RBD of the S protein of SARS-CoV-2 induces protective immunity. Nature, 1-9 (2020).
55. J. R. Francica, D. E. Zak, C. Linde, E. Siena, C. Johnson, M. Juraska, N. L. Yates, B. Gunn, E. D. Gregorio, B. J. Flynn, N. M. Valiante, P. Malyala, S. W. Barnett, P. Sarkar, M. Singh, S. Jain, M. Ackerman, M. Alam, G. Ferrari, A. Salazar, G. D. Tomaras, D. T. O'Hagan, A. Aderem, G. Alter, R. A. Seder, Innate transcriptional effects by adjuvants on the magnitude, quality, and durability of HIV envelope responses in NHPs. Blood Adv. 1, 2329-2342 (2017).
56. N. Doria-Rose, M. S. Suthar, M. Makowski, S. O'Connell, A. B. McDermott, B. Flach, J. E. Ledgerwood, J. R. Mascola, B. S. Graham, B. C. Lin, S. O'Dell, S. D. Schmidt, A. T. Widge, V.-V. Edara, E. J. Anderson, L. Lai, K. Floyd, N. G. Rouphael, V. Zarnitsyna, P. C. Roberts, M. Makhene, W. Buchanan, C. J. Luke, J. H. Beigel, L. A. Jackson, K. M. Neuzil, H. Bennett, B. Leav, J. Albert, P. Kunwar, mRNA-1273 S. Group, Antibody Persistence through 6 Months after the Second Dose of mRNA-1273 Vaccine for Covid-19. New Engl J Med (2021), doi:10.1056/nejmc2103916.
57. G. Mutwiri, V. Gerdts, S. van D. L. den Hurk, G. Auray, N. Eng, S. Garlapati, L. A. Babiuk, A. Potter, Combination adjuvants: the next generation of adjuvants? Expert Rev Vaccines. 10, 95-107 (2014).
58. L. E. Crowell, A. E. Lu, K. R. Love, A. Stockdale, S. M. Timmick, D. Wu, Y. (Annie) Wang, W. Doherty, A. Bonnyman, N. Vecchiarello, C. Goodwine, L. Bradbury, J. R. Brady, J. J. Clark, N. A. Colant, A. Cvetkovic, N. C. Dalvie, D. Liu, Y. Liu, C. A. Mascarenhas, C. B. Matthews, N. J. Mozdzierz, K. A. Shah, S.-L. Wu, W. S. Hancock, R. D. Braatz, S. M. Cramer, J. C. Love, On-demand manufacturing of clinical-quality biopharmaceuticals. Nat Biotechnol. 36, 988-995 (2018).
59. Z. Yang, W. Kong, Y. Huang, A. Roberts, B. R. Murphy, K. Subbarao, G. J. Nabel, A DNA vaccine induces SARS coronavirus neutralization and protective immunity in mice. Nature. 428, 561-564 (2004).
60. J. Yu, Z. Li, X. He, M. S. Gebre, E. A. Bondzie, H. Wan, C. Jacob-Dolan, D. R. Martinez, J. P. Nkolola, R. S. Baric, D. H. Barouch, Deletion of the SARS-CoV-2 Spike Cytoplasmic Tail Increases Infectivity in Pseudovirus Neutralization Assays. J Virol. 95 (2021), doi:10.1128/jvi.00044-21.
61. C. R. Moderbacher, S. I. Ramirez, J. M. Dan, A. Grifoni, K. M. Hastie, D. Weiskopf, S. Belanger, R. K. Abbott, C. Kim, J. Choi, Y. Kato, E. G. Crotty, C. Kim, S. A. Rawlings, J. Mateus, L. P. V. Tse, A. Frazier, R. Baric, B. Peters, J. Greenbaum, E. O. Saphire, D. M. Smith, A. Sette, S. Crotty, Antigen-Specific Adaptive Immunity to SARS-CoV-2 in Acute COVID-19 and Associations with Age and Disease Severity. Cell. 183, 996-1012.e19 (2020).

1. A composition, comprising:
(a) alum; and
(b) a SARS-CoV-2 Spike (S) glycoprotein variant, wherein the S glycoprotein variant comprises:
(i) a receptor binding domain (RBD) having a mutation of at least one amino acid residue in an angiotensin-converting enzyme 2 (ACE2) receptor binding motif (RBM) relative to a wild-type RBD, wherein the residue is (i) hydrophobic; and (ii) within an aggregation-prone region of about 3-15 amino acid residues, wherein the mutation is a substitution of the hydrophobic residue with a different amino acid residue; and
(ii) at least one linker comprising 2-12 phosphoserine residues, wherein the S glycoprotein variant is covalently bound to the alum via the phosphoserine residues.

2.-5. (canceled)

6. The composition of m claim 1, wherein the alum comprises a salt of aluminum.

7. The composition of claim 1, wherein the alum comprises aluminum hydroxide, aluminum phosphate, aluminum potassium sulfate, or combinations thereof.

8.-20. (canceled)

21. The composition of claim 1, wherein the S glycoprotein variant comprises an RBD having a mutation of at least one amino acid residue in a first and/or second aggregation-prone region relative to a wild-type RBD comprising the amino acid sequence of SEQ ID NO: 1, wherein the first aggregation-prone region comprises amino acid residues 122-126 of SEQ ID NO: 1, and the second aggregation-prone region comprises amino acid residues 158-162 of SEQ ID NO: 1, and wherein the mutation is a substitution with a different amino acid residue.

22.-70. (canceled)

71. The composition of claim 1, further comprising a non-liposome, non-micelle particle, wherein the particle comprises a lipid, a sterol, a saponin, and an optional additional non-alum adjuvant, wherein the particle is optionally bound to the alum

72. The composition of claim 71, wherein the particle is a porous, cage-like nanoparticle.

73. (canceled)

74. The composition of claim 71, wherein the lipid is a phospholipid, optionally wherein the phospholipid is 2-Dipalmitoyl-snglycero-3-phosphocholine (DPPC).

75. (canceled)

76. The composition of claim 71, wherein the sterol comprises cholesterol or a derivative thereof.

77. The composition of claim 71, wherein the saponin is a natural or synthetic saponin.

78. The composition of claim 77, wherein the saponin comprises Quil A® or a submixture or pure saponin separated therefrom, or a natural or synthetic Q-21 or an analog thereof.

79.-88. (canceled)

89. The composition of claim 71 comprising a lipid:additional adjuvant:sterol:saponin molar ratio of 2.5:1:10:10, or a variation thereof wherein the molar ratio of lipid, additional adjuvant, sterol, saponin or any combination thereof is increased or decreased by any value between about 0 and about 3.

90. The composition of claim 89, wherein
(a) the DPPC:MPLA:cholesterol:Quil A®
are in a molar ratio of 2.5:1:10:10; or
(b) the Quil-A®; chol:DPPC,NPLA are in a mass ratio of 10:2:1:1.

91. (canceled)

92. The composition of claim 71, wherein the alum and the particle are bound, optionally wherein the particle is covalently bound to the alum via phosphate residues in the particle.

93. (canceled)

94. The composition of claim 71, wherein a molar ratio of alum:particle is between about 1:500 and about 500:1; and/or a molar ratio of alum:S glycoprotein variant is between about 1:500 and about 500:1.

95. (canceled)

96. A pharmaceutical composition comprising the composition of claim 1 and a pharmaceutically acceptable carrier.

97. A vaccine comprising the composition of claim 1.

98. A method for
(a) generating an immune response against a S glycoprotein variant, comprising administering to a subject an amount effective to generate an immune response in the subject of the composition or vaccine of claim 1;
(b) treating a subject in need thereof comprising administering to a subject infected with SARS-CoV-2 the composition or vaccine of claim 1 in an effective amount to induce an immune response against the S glycoprotein variant; or
(c) limiting SARS-CoV-2 infection in a subject comprising administering to a subject at risk for being exposed to and/or infected by SARS-CoV-2 the composition or vaccine of claim 1 in an effective amount to induce an immune response against the S glycoprotein variant.

99.-106. (canceled)

107. A nucleic acid encoding the S glycoprotein variant and at least one linker comprising 2-12 phosphoserine residue.

108. An expression vector comprising the nucleic acid of claim 107 operatively linked to a suitable control sequence.

109. A host cell comprising the expression vector of claim 108.


SEQUENCE LISTING (124 sequences)

SEQ ID NO: 1 - protein, 202 aa, synthetic construct
  REGION 1..202: note = Synthetic
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 2 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 3 - protein, 202 aa, synthetic construct
  REGION 1..202: note = Synthetic
NITNLCPFGEVFQATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 4 - protein, 201 aa, synthetic construct
  REGION 1..201: note = Synthetic
ITNLCPFGEVFQATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 5 - protein, 240 aa, synthetic construct
  REGION 1..240: note = Synthetic
ITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGGSLEVLFQGPGSHHHHHHHHHH

SEQ ID NO: 6 - protein, 201 aa, synthetic construct
  REGION 1..201: note = Synthetic
ITNLCPFGEVFNATRFASVYAWNRKRISNCVADYSVAYNSASFSTFKCYGVSPTKLNDLCFTNVYADSFVIRGDEVRQIAPGQTGKIADYNYKLPDDFTGCVIAWNSNNLDSKVGGNYNYKYRYLRKSNLKPFERDISTEIYQAGSTPCNGVEGFNCYWPLQSYGFQPTNGVGYQPYRVVVLSFEAAHAPATVCGPKKSTN

SEQ ID NO: 7 - protein, 201 aa, synthetic construct
  REGION 1..201: note = Synthetic
ITNLCPFGEVFNATRFASVYAWNRKRISNCVADYSVAYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFEAAHAPATVCGPKKSTN

SEQ ID NO: 8 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRYLRKSNLKPFERDISTEIYQAGSTPCNGVEGFNCYWPLQSYGFQPTNGVGYQPYRVVVLSFELLHAPATVCGPKKSTN

SEQ ID NO: 9 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTN

SEQ ID NO: 10 - protein, 201 aa, synthetic construct
  REGION 1..201: note = Synthetic
ITNLCPFGEVFNATRFASVYAWNRKRISNCVADYSVAYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFEAAHAPATVCGPKKSTN

SEQ ID NO: 11 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 12 - protein, 201 aa, synthetic construct
  REGION 1..201: note = Synthetic
ITNLCPFGEVFNATRFASVYAWNRKRISNCVADYSVMYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 13 - protein, 201 aa, synthetic construct
  REGION 1..201: note = Synthetic
ITNLCPFGEVFNATRFASVYAWNRKRISNCVADYSVMYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELMHAPATVCGPKKSTN

SEQ ID NO: 14 - protein, 201 aa, synthetic construct
  REGION 1..201: note = Synthetic
ITNLCPFGEVFNATRFASVYAWNRKRISNCVADYSVMYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELMHAPATVCGPKKSTN

SEQ ID NO: 15 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCRFPLQSYGFQPTNGVGYQPYRVVVLSFELLHAPATVCGPKKSTN

SEQ ID NO: 16 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYMPLQSYGFQPTNGVGYQPYRVVVLSFELLHAPATVCGPKKSTN

SEQ ID NO: 17 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCRMPLQSYGFQPTNGVGYQPYRVVVLSFELLHAPATVCGPKKSTN

SEQ ID NO: 18 - protein, 201 aa, synthetic construct
  REGION 1..201: note = Synthetic
ITNLCPFGEVFNATRFASVYAWNRKRISNCVADYSVMYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCRMPLQSYGFQPTNGVGYQPYRVVVLSFELMHAPATVCGPKKSTN

SEQ ID NO: 19 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRYLRKSNLKPFERDISTEIYQAGSTPCNGVEGFNCYFPLQSYGFQPTNGVGYQPYRVVVLSFELLHAPATVCGPKKSTN

SEQ ID NO: 20 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRYLRKSNLKPFERDISTEIYQAGSTPCNGVEGFNCYFPLQSYGFQPTNGVGYQPYRVVVLSFELLHAPATVCGPKKSTN

SEQ ID NO: 21 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRSLRKSNLKPFERDISTEIYQAGSTPCNGVEGFNCYFPLQSYGFQPTNGVGYQPYRVVVLSFELLHAPATVCGPKKSTN

SEQ ID NO: 22 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRSLRKSNLKPFERDISTEIYQAGSTPCNGVEGFNCYFPLQSYGFQPTNGVGYQPYRVVVLSFELLHAPATVCGPKKSTN

SEQ ID NO: 23 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYAYRLFRKSNLKPFERDISTEIYQAGSTPCNGVEGFNCYFPLQSYGFQPTNGVGYQPYRVVVLSFELLHAPATVCGPKKSTN

SEQ ID NO: 24 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCVRPLQSYGFQPTNGVGYQPYRVVVLSFELLHAPATVCGPKKSTN

SEQ ID NO: 25 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYNPLQSYGFQPTNGVGYQPYRVVVLSFELLHAPATVCGPKKSTN

SEQ ID NO: 26 - protein, 201 aa, synthetic construct
  REGION 1..201: note = Synthetic
ITNLCPFGEVFNATRFASVYAWERKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 27 - protein, 201 aa, synthetic construct
  REGION 1..201: note = Synthetic
ITNLCPFGEVFNATRFASVYAWNRKRISNCVADWSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 28 - protein, 201 aa, synthetic construct
  REGION 1..201: note = Synthetic
ITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 29 - protein, 201 aa, synthetic construct
  REGION 1..201: note = Synthetic
ITNLCPFGEAFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 30 - protein, 201 aa, synthetic construct
  REGION 1..201: note = Synthetic
ITNLCPFGEVFNATRFPSVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 31 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGVIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 32 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELDHAPATVCGPKKSTN

SEQ ID NO: 33 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATRCGPKKSTN

SEQ ID NO: 34 - protein, 201 aa, synthetic construct
  REGION 1..201: note = Synthetic
ITNLCDFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 35 - protein, 201 aa, synthetic construct
  REGION 1..201: note = Synthetic
ITNLCPFGEVFNATRFASVYAWNRKKISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 36 - protein, 201 aa, synthetic construct
  REGION 1..201: note = Synthetic
ITNLCPFGEVFNATRFASVYAWNRKRISNCVADYSVLYNSASFSTFKCYGVDPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 37 - protein, 201 aa, synthetic construct
  REGION 1..201: note = Synthetic
ITNLCPFGEVFNATRFASVYAWNRKRISNCVADYSVLYNSASFSTFKCYGVSPTKLNDYCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 38 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCWTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 39 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVDQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 40 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGATGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 41 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDNFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 42 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNIDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 43 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPHRVVVLSFELLHAPATVCGPKKSTN

SEQ ID NO: 44 - protein, 201 aa, synthetic construct
  REGION 1..201: note = Synthetic
ITNLCPFGEVFNATRFASVYAWNRKRISNCVADYSVLYNSASFSTFRCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 45 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVVRGDEVRQIAPGQTGKIADYNYKLPDDFTGCVIAWNSNNLDSKVGGNYNYKYRLFRKSNLKPFERDISTEIYQAGSTPCNGVEGFNCYFPLQSYGFQPTNGVGYQPYRVVVLSFELLHAPATVCGPKKSTN

SEQ ID NO: 46 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYFPLDSYGFQPTNGVGYQPYRVVVLSFELLHAPATVCGPKKSTN

SEQ ID NO: 47 - protein, 201 aa, synthetic construct
  REGION 1..201: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRNFRKSNLKPFERDISTEIYQAGSTPCNGVEGFNCYFPLQSYGFQPTNGVGYQPYRVVVLSFELLHAPATVCGPKKSTN

SEQ ID NO: 48 - protein, 208 aa, synthetic construct
  REGION 1..208: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNHHHHHHC

SEQ ID NO: 49 - protein, 225 aa, synthetic construct
  REGION 1..225: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGGDGGDGGDGGAHIVMVDAYKPTK

SEQ ID NO: 50 - protein, 225 aa, synthetic construct
  REGION 1..225: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTNGGDGGDGGDGGAHIVMVDAYKPTK

SEQ ID NO: 51 - protein, 207 aa, synthetic construct
  REGION 1..207: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYWPLQSYGFQPTNGVGYQPYRVVVLSFELLHAPATVCGPKKSTNLVKNKC

SEQ ID NO: 52 - protein, 207 aa, synthetic construct
  REGION 1..207: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTNLVKNKC

SEQ ID NO: 53 - protein, 259 aa, synthetic construct
  REGION 1..259: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYWPLQSYGFQPTNGVGYQPYRVVVLSFELLHAPATVCGPKKSTNLVKNKSVNFNFNGLTGTGVLTESNKKFLPFQQFGRDIADTTDAVRDPQTLEILDITPC

SEQ ID NO: 54 - protein, 262 aa, synthetic construct
  REGION 1..262: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYWPLQSYGFQPTNGVGYQPYRVVVLSFELLHAPATVCGPKKSTNLVKNKSVNFNFNGLTGTGVLTESNKKFLPFQQFGRDIADTTDAVRDPQTLEILDITPCPPC

SEQ ID NO: 55 - protein, 259 aa, synthetic construct
  REGION 1..259: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTNLVKNKSVNFNFNGLTGTGVLTESNKKFLPFQQFGRDIADTTDAVRDPQTLEILDITPC

SEQ ID NO: 56 - protein, 262 aa, synthetic construct
  REGION 1..262: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTNLVKNKSVNFNFNGLTGTGVLTESNKKFLPFQQFGRDIADTTDAVRDPQTLEILDITPCPPC

SEQ ID NO: 57 - protein, 260 aa, synthetic construct
  REGION 1..260: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYWPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 58 - protein, 260 aa, synthetic construct
  REGION 1..260: note = Synthetic
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 59 - protein, 1272 aa, synthetic construct
  REGION 1..1272: note = Synthetic
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 60 - protein, 12 aa, synthetic construct
  REGION 1..12: note = Synthetic
FVFLVLLPLVSS

SEQ ID NO: 61 - protein, 1260 aa, synthetic construct
  REGION 1..1260: note = Synthetic
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 62 - DNA, 606 bp, synthetic construct
  misc_feature 1..606: note = Synthetic
  source 1..606: mol_type = other DNA
aacatcaccaacttgtgcccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 63 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 64 - DNA, 606 bp, synthetic construct
  misc_feature 1..606: note = Synthetic
  source 1..606: mol_type = other DNA
aacatcaccaacttgtgcccattcggtgaggttttccaggctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcaccaacgtttacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgctacttcccattgcagtcttacggtttccagccaactaacggtgtcggttaccaaccatacagagttgtcgttttgtccttcgagttgttgcacgctccagctactgtttgtggtccaaagaagtccactaac

SEQ ID NO: 65 - DNA, 603 bp, synthetic construct
  misc_feature 1..603: note = Synthetic
  source 1..603: mol_type = other DNA
atcaccaacttgtgtccattcggtgaggttttccaggctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcaccaacgtttacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgctacttcccattgcagtcttacggtttccagccaactaacggtgtcggttaccaaccatacagagttgtcgttttgtccttcgagttgttgcacgctccagctactgtttgtggtccaaagaagtccactaac

SEQ ID NO: 66 - DNA, 720 bp, synthetic construct
  misc_feature 1..720: note = Synthetic
  source 1..720: mol_type = other DNA
atcaccaacttgtgtccattcggtgaggttttcaacgccactaagttcccatctgtttacgcctgggagagaaagaagatctccaactgcgttgctgactactccgtcttgtacaactccacattcttcagcaccttcaagtgctacggtgtttccgctactaagttgaacgacctgtgtttctccaacgtttacgccgactccttcgttgttaagggtgacgacgttagacagattgctccaggtcagactggtgttatcgctgactacaactacaagctgccagacgacttcatgggttgtgttttggcttggaacaccagaaacatcgacgctacttccaccggtaattacaattacaagtaccgttacctgaggcacggtaagcttagaccattcgagagagacatctctaacgtcccattctctccagacggtaagccttgtactcctcctgctttgaactgttactggccattgaacgactacggtttctacactactaccggtatcggttaccagccatacagagttgttgtcttgtccttcgagttgttgaacgctccagctactgtttgcggtccaaagttgtccaccgacctgattaagaaccagtgcgtcaactttaacttcaacggtctgactggtactggtggttccttggaggttttgtttcaaggtccaggttctcatcaccaccaccatcaccatcatcaccac

SEQ ID NO: 67 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcgcttacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgttatttgagaaagtccaacctgaagccattcgagagagacatctccactgagatctaccaagctggttccactccatgtaacggtgttgagggtttcaactgttactggccattgcagtcctacggtttccagccaactaatggtgttggttaccagccatacagagtcgtcgttttgtccttcgaggctgctcatgctccagctactgtttgcggtccaaagaagtccactaac

SEQ ID NO: 68 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcgcttacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtttgtttagaaagtccaacctgaagccattcgagagagacatctccactgagatctaccaagctggttccactccatgtaacggtgttgagggtttcaactgttactggccattgcagtcctacggtttccagccaactaatggtgttggttaccagccatacagagtcgtcgttttgtccttcgaggctgctcatgctccagctactgtttgcggtccaaagaagtccactaac

SEQ ID NO: 69 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgttatttgagaaagtccaacctgaagccattcgagagagacatctccactgagatctaccaagctggttccactccatgtaacggtgttgagggtttcaactgttactggccattgcagtcctacggtttccagccaactaatggtgttggttaccagccatacagagtcgtcgttttgtccttcgagttgttgcatgctccagctactgtttgcggtccaaagaagtccactaac

SEQ ID NO: 70 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtttgtttagaaagtccaacctgaagccattcgagagagacatctccactgagatctaccaagctggttccactccatgtaacggtgttgagggtttcaactgttactggccattgcagtcctacggtttccagccaactaatggtgttggttaccagccatacagagtcgtcgttttgtccttcgagttgttgcatgctccagctactgtttgcggtccaaagaagtccactaac

SEQ ID NO: 71 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcgcttacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacttgtaccgtttgtttagaaagtccaacctgaagccattcgagagagacatctccactgagatctaccaagctggttccactccatgtaacggtgttgagggtttcaactgttactttccattgcagtcctacggtttccagccaactaatggtgttggttaccagccatacagagtcgtcgttttgtccttcgaggctgctcatgctccagctactgtttgcggtccaaagaagtccactaac

SEQ ID NO: 72 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 73 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcatgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 74 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcatgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgatgcatgctccagctactgtttgcggtccaaagaagtccactaac

SEQ ID NO: 75 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcatgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgatgcatgctccagctactgtttgcggtccaaagaagtccactaac

SEQ ID NO: 76 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgtagattcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 77 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacatgccattgcagtcctacggtttccagccaactaatggtgttggttaccagccatacagagtcgtcgttttgtccttcgagttgttgcatgctccagctactgtttgcggtccaaagaagtccactaac

SEQ ID NO: 78 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgtagaatgccattgcagtcctacggtttccagccaactaatggtgttggttaccagccatacagagtcgtcgttttgtccttcgagttgttgcatgctccagctactgtttgcggtccaaagaagtccactaac

SEQ ID NO: 79 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcatgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgtagaatgccattgcagtcctacggtttccagccaactaatggtgttggttaccagccatacagagtcgtcgttttgtccttcgagttgatgcatgctccagctactgtttgcggtccaaagaagtccactaac

SEQ ID NO: 80 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgttacctgagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 81 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaattacaattacaagtaccgttacctgaggaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 82 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtacagatccctgagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 83 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaattacaattacaagtacaggtccctgaggaagtccaacttgaagccattcgagagagacatctccaccgagatctaccaagctggttctactccatgtaacggtgtcgagggtttcaactgttacttcccattgcagtcctacggtttccagccaactaacggtgttggttaccagccatacagagttgtcgttttgtccttcgagttgttgcacgctccagctactgtttgtggtccaaagaagtccactaac

SEQ ID NO: 84 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacgcctacaggctgttcagaaagtccaacttgaagccattcgagagagacatctccaccgagatctaccaagctggttctactccatgtaacggtgtcgagggtttcaactgttacttcccattgcagtcctacggtttccagccaactaacggtgttggttaccagccatacagagttgtcgttttgtccttcgagttgttgcacgctccagctactgtttgtggtccaaagaagtccactaac

SEQ ID NO: 85 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaattacaattacaagtacaggctgttccgtaagtccaacctgaagccattcgagagagacatctccactgagatctaccaagctggttccactccatgtaacggtgttgagggtttcaactgcgtcagaccattgcaatcttacggtttccagccaactaacggtgtcggttaccaaccatacagagttgtcgttttgtccttcgagttgttgcacgctccagctactgtttgtggtccaaagaagtccactaac

SEQ ID NO: 86 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaattacaattacaagtacaggctgttccgtaagtccaacctgaagccattcgagagagacatctccactgagatctaccaagctggttccactccatgtaacggtgttgagggtttcaactgttacaacccattgcagtcctacggtttccagccaactaatggtgttggttaccagccatacagagtcgtcgttttgtccttcgagttgttgcatgctccagctactgtttgcggtccaaagaagtccactaac

SEQ ID NO: 87 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctgggaaagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 88 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactggtccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 89 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactcctttttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 90 - DNA, 603 bp, synthetic construct
  misc_feature 1..603: note = Synthetic
  source 1..603: mol_type = other DNA
atcaccaacttgtgtccattcggtgagattttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 91 - DNA, 603 bp, synthetic construct
  misc_feature 1..603: note = Synthetic
  source 1..603: mol_type = other DNA
atcaccaacttgtgtccattcggtgaggttttcaacgctactagattcccatccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 92 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtgttatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 93 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttggatcatgctccagctactgtttgcggtccaaagaagtccactaac

SEQ ID NO: 94 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactagatgcggtccaaagaagtccactaac

SEQ ID NO: 95 - DNA, 603 bp, synthetic construct
  misc_feature 1..603: note = Synthetic
  source 1..603: mol_type = other DNA
atcaccaacttgtgtgatttcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 96 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagaaaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 97 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgttgatccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 98 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgactattgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 99 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgttggactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 100 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttgatcagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 101 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtgctactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 102 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacaatttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 103 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacattgactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 104 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccacatagagtcgtcgttttgtccttcgagttgttgcatgctccagctactgtttgcggtccaaagaagtccactaac

SEQ ID NO: 105 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcagatgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 106 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttgttagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 107 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtctgttcagaaagtccaacctgaagccattcgagagagacatctccactgagatctaccaagctggttccactccatgtaacggtgttgagggtttcaactgttacttcccattgcagtcctacggtttcgatccaactaatggtgttggttaccagccatacagagtcgtcgttttgtccttcgagttgttgcatgctccagctactgtttgcggtccaaagaagtccactaac

SEQ ID NO: 108 - DNA, 603 bp, synthetic construct
  misc_feature 1..603: note = Synthetic
  source 1..603: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtaatttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaac

SEQ ID NO: 109 - DNA, 624 bp, synthetic construct
  misc_feature 1..624: note = Synthetic
  source 1..624: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaaccatcatcatcatcatcattgt

SEQ ID NO: 110 - DNA, 675 bp, synthetic construct
  misc_feature 1..675: note = Synthetic
  source 1..675: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaaatctaccaatggtggtgatggtggcgacggcggagatggtggtgctcatatagttatggttgacgcctacaagcctactaag

SEQ ID NO: 111 - DNA, 675 bp, synthetic construct
  misc_feature 1..675: note = Synthetic
  source 1..675: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaattacaattacaagtacaggctgttccgtaagtccaacctgaagccattcgagagagacatctccactgagatctaccaagctggttccactccatgtaacggtgttgagggtttcaactgttactggccattgcagtcttacggtttccagccaactaacggtgtcggttaccaaccatacagagttgtcgttttgtccttcgagttgttgcacgctccagctactgtttgtggtccaaagaaatccaccaatggtggtgatggtggcgacggcggagatggtggtgctcatatagttatggttgacgcctacaagcctactaag

SEQ ID NO: 112 - DNA, 621 bp, synthetic construct
  misc_feature 1..621: note = Synthetic
  source 1..621: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaacttggtcaagaacaaatgt

SEQ ID NO: 113 - DNA, 621 bp, synthetic construct
  misc_feature 1..621: note = Synthetic
  source 1..621: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtttgtttagaaagtccaacctgaagccattcgagagagacatctccactgagatctaccaagctggttccactccatgtaacggtgttgagggtttcaactgttactggccattgcagtcctacggtttccagccaactaatggtgttggttaccagccatacagagtcgtcgttttgtccttcgagttgttgcatgctccagctactgtttgcggtccaaagaagtccactaacttggtcaagaacaaatgt

SEQ ID NO: 114 - DNA, 777 bp, synthetic construct
  misc_feature 1..777: note = Synthetic
  source 1..777: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaacttggtcaagaacaaatctgtcaactttaacttcaacggcctgaccggtactggtgttttgactgaatccaacaagaagttcctgccattccagcagttcggtagagacattgctgacactactgacgccgttagagatccacagactttggagatcttggacatcaccccatgt

SEQ ID NO: 115 - DNA, 786 bp, synthetic construct
  misc_feature 1..786: note = Synthetic
  source 1..786: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaacttggtcaagaacaaatctgtcaactttaacttcaacggcctgaccggtactggtgttttgactgaatccaacaagaagttcctgccattccagcagttcggtagagacattgctgacactactgacgccgttagagatccacagactttggagatcttggacatcaccccatgtccaccatgt

SEQ ID NO: 116 - DNA, 777 bp, synthetic construct
  misc_feature 1..777: note = Synthetic
  source 1..777: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtttgtttagaaagtccaacctgaagccattcgagagagacatctccactgagatctaccaagctggttccactccatgtaacggtgttgagggtttcaactgttactggccattgcagtcctacggtttccagccaactaatggtgttggttaccagccatacagagtcgtcgttttgtccttcgagttgttgcatgctccagctactgtttgcggtccaaagaagtccactaacttggtcaagaacaaatctgtcaactttaacttcaacggcctgaccggtactggtgttttgactgaatccaacaagaagttcctgccattccagcagttcggtagagacattgctgacactactgacgccgttagagatccacagactttggagatcttggacatcaccccatgt

SEQ ID NO: 117 - DNA, 786 bp, synthetic construct
  misc_feature 1..786: note = Synthetic
  source 1..786: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacaaataccgtttgtttagaaagtccaacctgaagccattcgagagagacatctccactgagatctaccaagctggttccactccatgtaacggtgttgagggtttcaactgttactggccattgcagtcctacggtttccagccaactaatggtgttggttaccagccatacagagtcgtcgttttgtccttcgagttgttgcatgctccagctactgtttgcggtccaaagaagtccactaacttggtcaagaacaaatctgtcaactttaacttcaacggcctgaccggtactggtgttttgactgaatccaacaagaagttcctgccattccagcagttcggtagagacattgctgacactactgacgccgttagagatccacagactttggagatcttggacatcaccccatgtccaccatgt

SEQ ID NO: 118 - DNA, 780 bp, synthetic construct
  misc_feature 1..780: note = Synthetic
  source 1..780: mol_type = other DNA
atcaccaacttgtgtccattcggtgaggttttcaacgctactagattcgcttccgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacctgaagccattcgagagagacatctccactgagatctaccaagctggttccactccatgtaacggtgttgagggtttcaactgttacttcccattgcagtcctacggtttccagccaactaatggtgttggttaccagccatacagagtcgtcgttttgtccttcgagttgttgcatgctccagctactgtttgcggtccaaagaagtccactaacctggtcaagaacaagtgcgtcaactttaacttcaacggtctgaccggtactggtgttttgactgagtccaacaagaagttcctgccattccagcaattcggtagagacattgctgacactactgacgccgttagagatccacagactttggagatcttggacatcaccccatgttct

SEQ ID NO: 119 - DNA, 780 bp, synthetic construct; identical to SEQ ID NO: 118
  misc_feature 1..780: note = Synthetic
  source 1..780: mol_type = other DNA

SEQ ID NO: 120 - protein, 4 aa, synthetic construct
  REGION 1..4: note = Synthetic
  REGION 1..4: note = MISC_FEATURE - phosphoserine
SSSS

SEQ ID NO: 121 - protein, 8 aa, synthetic construct
  REGION 1..8: note = Synthetic
  REGION 1..8: note = MISC_FEATURE - phosphoserine
SSSSSSSS

SEQ ID NO: 122 - protein, 208 aa, synthetic construct; identical to SEQ ID NO: 48
  REGION 1..208: note = Synthetic

SEQ ID NO: 123 - protein, 208 aa, synthetic construct
  REGION 1..208: note = Synthetic
CITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNHHHHHH

SEQ ID NO: 124 - protein, 202 aa, synthetic construct
  REGION 1..202: note = Synthetic
CITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTN